SEQUENCE LISTING (271 sequences)

SEQ ID NO: 1 - DNA, 25 bp, synthetic construct
  source 1..25: mol_type = other DNA
tctggagatgattcaggaagtgtct

SEQ ID NO: 2 - DNA, 22 bp, synthetic construct
  source 1..22: mol_type = other DNA
cgcacggcagaaaggatatctt

SEQ ID NO: 3 - DNA, 24 bp, synthetic construct
  source 1..24: mol_type = other DNA
ctcaatctacagacgcctctacag

SEQ ID NO: 4 - DNA, 26 bp, synthetic construct
  source 1..26: mol_type = other DNA
ctgtttcggatatatcgccattgact

SEQ ID NO: 5 - DNA, 24 bp, synthetic construct
  source 1..24: mol_type = other DNA
ctcaatctacagacgcctctacag

SEQ ID NO: 6 - DNA, 26 bp, synthetic construct
  source 1..26: mol_type = other DNA
ctgtttcggatatatcgccattgact

SEQ ID NO: 7 - DNA, 25 bp, synthetic construct
  source 1..25: mol_type = other DNA
tctggagatgattcaggaagtgtct

SEQ ID NO: 8 - DNA, 22 bp, synthetic construct
  source 1..22: mol_type = other DNA
cgcacggcagaaaggatatctt

SEQ ID NO: 9 - DNA, 25 bp, synthetic construct
  source 1..25: mol_type = other DNA
tggcgatatatccgaaacagaatcc

SEQ ID NO: 10 - DNA, 22 bp, synthetic construct
  source 1..22: mol_type = other DNA
cgcacggcagaaaggatatctt

SEQ ID NO: 11 - DNA, 24 bp, synthetic construct
  source 1..24: mol_type = other DNA
gcagcagcactcaattctctaaga

SEQ ID NO: 12 - DNA, 19 bp, synthetic construct
  source 1..19: mol_type = other DNA
ggtccagggccgaatacac

SEQ ID NO: 13 - DNA, 19 bp, synthetic construct
  source 1..19: mol_type = other DNA
ctcaagccgggcctagtag

SEQ ID NO: 14 - DNA, 20 bp, synthetic construct
  source 1..20: mol_type = other DNA
agaggatggcgttggtgatg

SEQ ID NO: 15 - DNA, 24 bp, synthetic construct
  source 1..24: mol_type = other DNA
cgcctctacagatacaagcgatat

SEQ ID NO: 16 - DNA, 21 bp, synthetic construct
  source 1..21: mol_type = other DNA
ccagctccgtcagagtctttg

SEQ ID NO: 17 - DNA, 25 bp, synthetic construct
  source 1..25: mol_type = other DNA
cagaacatgctcttcatcctaaagc

SEQ ID NO: 18 - DNA, 22 bp, synthetic construct
  source 1..22: mol_type = other DNA
gctaagtcaggatggcccattt

SEQ ID NO: 19 - DNA, 25 bp, synthetic construct
  source 1..25: mol_type = other DNA
cagaacatgctcttcatcctaaagc

SEQ ID NO: 20 - DNA, 21 bp, synthetic construct
  source 1..21: mol_type = other DNA
gctaagtcaggatggcccatt

SEQ ID NO: 21 - DNA, 25 bp, synthetic construct
  source 1..25: mol_type = other DNA
gctttgcaagagccctataatgaac

SEQ ID NO: 22 - DNA, 27 bp, synthetic construct
  source 1..27: mol_type = other DNA
ctaggatgattagggtgttgtttacgt

SEQ ID NO: 23 - DNA, 26 bp, synthetic construct
  source 1..26: mol_type = other DNA
actatattcatcacatgtgcgaagct

SEQ ID NO: 24 - DNA, 22 bp, synthetic construct
  source 1..22: mol_type = other DNA
gcagaagaggcttttcgctttt

SEQ ID NO: 25 - DNA, 26 bp, synthetic construct
  source 1..26: mol_type = other DNA
caagaccttttttcgctgtttttgtc

SEQ ID NO: 26 - DNA, 25 bp, synthetic construct
  source 1..25: mol_type = other DNA
ccccttctaaaagagctcgaatgat

SEQ ID NO: 27 - DNA, 25 bp, synthetic construct
  source 1..25: mol_type = other DNA
gatgtcgcttctcgctatcatatca

SEQ ID NO: 28 - DNA, 26 bp, synthetic construct
  source 1..26: mol_type = other DNA
tgactccgtatttctctgctatctct

SEQ ID NO: 29 - DNA, 25 bp, synthetic construct
  source 1..25: mol_type = other DNA
ggtgggattagtggtgataggacta

SEQ ID NO: 30 - DNA, 28 bp, synthetic construct
  source 1..28: mol_type = other DNA
agcaggaatagctagatagatgggattt

SEQ ID NO: 31 - DNA, 25 bp, synthetic construct
  source 1..25: mol_type = other DNA
ggtgggattagtggtgataggacta

SEQ ID NO: 32 - DNA, 28 bp, synthetic construct
  source 1..28: mol_type = other DNA
agcaggaatagctagatagatgggattt

SEQ ID NO: 33 - DNA, 20 bp, synthetic construct
  source 1..20: mol_type = other DNA
ggtggtgcgctttatgcaaa

SEQ ID NO: 34 - DNA, 25 bp, synthetic construct
  source 1..25: mol_type = other DNA
ccgcagtctccagacatattgttaa

SEQ ID NO: 35 - DNA, 22 bp, synthetic construct
  source 1..22: mol_type = other DNA
gatagcaataccgctgctcatg

SEQ ID NO: 36 - DNA, 25 bp, synthetic construct
  source 1..25: mol_type = other DNA
cccaggcagcagagttatttagaaa

SEQ ID NO: 37 - DNA, 28 bp, synthetic construct
  source 1..28: mol_type = other DNA
ttttactcagttttatcccaggcttctt

SEQ ID NO: 38 - DNA, 30 bp, synthetic construct
  source 1..30: mol_type = other DNA
agtcataccaaagactaaaaagcgttatca

SEQ ID NO: 39 - DNA, 28 bp, synthetic construct
  source 1..28: mol_type = other DNA
ttttactcagttttatcccaggcttctt

SEQ ID NO: 40 - DNA, 30 bp, synthetic construct; identical to SEQ ID NO: 38
  source 1..30: mol_type = other DNA

SEQ ID NO: 41 - DNA, 26 bp, synthetic construct
  source 1..26: mol_type = other DNA
ccaggtgtgaattgtatggtttgaaa

SEQ ID NO: 42 - DNA, 25 bp, synthetic construct
  source 1..25: mol_type = other DNA
gtcttgtctacagagggcaatgatt

SEQ ID NO: 43 - DNA, 27 bp, synthetic construct
  source 1..27: mol_type = other DNA
ccgtcccctaatgaaatactgaaaaga

SEQ ID NO: 44 - DNA, 28 bp, synthetic construct
  source 1..28: mol_type = other DNA
cctcacttcattgcgcttataaataggt

SEQ ID NO: 45 - DNA, 24 bp, synthetic construct
  source 1..24: mol_type = other DNA
gctttaaaaccgcttgtcagttct

SEQ ID NO: 46 - DNA, 25 bp, synthetic construct
  source 1..25: mol_type = other DNA
ggcaattgggctaaacaaaaggtta

SEQ ID NO: 47 - DNA, 25 bp, synthetic construct
  source 1..25: mol_type = other DNA
attgttatcagccccttaagcttgt

SEQ ID NO: 48 - DNA, 28 bp, synthetic construct
  source 1..28: mol_type = other DNA
cttgaccaaaaacaccaataaagggtaa

SEQ ID NO: 49 - DNA, 26 bp, synthetic construct
  source 1..26: mol_type = other DNA
gaagattgcttgtgttgttcatgtca

SEQ ID NO: 50 - DNA, 25 bp, synthetic construct
  source 1..25: mol_type = other DNA
aacagtgtgaacaaccagtggaata

SEQ ID NO: 51 - DNA, 25 bp, synthetic construct
  source 1..25: mol_type = other DNA
ggtggatctgattcaatgctaatgc

SEQ ID NO: 52 - DNA, 25 bp, synthetic construct
  source 1..25: mol_type = other DNA
cacgtaatgaggttgatctggtgtt

SEQ ID NO: 53 - DNA, 25 bp, synthetic construct
  source 1..25: mol_type = other DNA
ccatacagtccgatatgccaataga

SEQ ID NO: 54 - DNA, 25 bp, synthetic construct
  source 1..25: mol_type = other DNA
gcctttgggtttacgtccataagaa

SEQ ID NO: 55 - DNA, 24 bp, synthetic construct
  source 1..24: mol_type = other DNA
gcattgccgcaaccaaattaaaag

SEQ ID NO: 56 - DNA, 26 bp, synthetic construct
  source 1..26: mol_type = other DNA
cgatataacttcgtagcgttcttgct

SEQ ID NO: 57 - DNA, 25 bp, synthetic construct
  source 1..25: mol_type = other DNA
cattgccgcaaccaaattaaaagaa

SEQ ID NO: 58 - DNA, 25 bp, synthetic construct
  source 1..25: mol_type = other DNA
gctatcacgagcgatataacttcgt

SEQ ID NO: 59 - DNA, 25 bp, synthetic construct
  source 1..25: mol_type = other DNA
gtagcaaatagtgatgtcgccgtat

SEQ ID NO: 60 - DNA, 25 bp, synthetic construct
  source 1..25: mol_type = other DNA
gcctttgggtttacgtccataagaa

SEQ ID NO: 61 - DNA, 24 bp, synthetic construct
  source 1..24: mol_type = other DNA
gatgtcgccgtattaacacccata

SEQ ID NO: 62 - DNA, 25 bp, synthetic construct
  source 1..25: mol_type = other DNA
gcctttgggtttacgtccataagaa

SEQ ID NO: 63 - DNA, 24 bp, synthetic construct
  source 1..24: mol_type = other DNA
gcattgccgcaaccaaattaaaag

SEQ ID NO: 64 - DNA, 26 bp, synthetic construct
  source 1..26: mol_type = other DNA
cgatataacttcgtagcgttcttgct

SEQ ID NO: 65 - DNA, 20 bp, synthetic construct
  source 1..20: mol_type = other DNA
cggcaaaaagacacggttgt

SEQ ID NO: 66 - DNA, 25 bp, synthetic construct
  source 1..25: mol_type = other DNA
gctatcacgagcgatataacttcgt

SEQ ID NO: 67 - DNA, 23 bp, synthetic construct
  source 1..23: mol_type = other DNA
ggcgcaacacaggattattttcc

SEQ ID NO: 68 - DNA, 23 bp, synthetic construct
  source 1..23: mol_type = other DNA
ttcagcgtgatgatgtcgatagg

SEQ ID NO: 69 - DNA, 21 bp, synthetic construct
  source 1..21: mol_type = other DNA
cggtacgttgggattgtgaac

SEQ ID NO: 70 - DNA, 22 bp, synthetic construct
  source 1..22: mol_type = other DNA
cgtctttggaaaccgcgatttt

SEQ ID NO: 71 - DNA, 21 bp, synthetic construct
  source 1..21: mol_type = other DNA
ttgccggatggaaacctgatt

SEQ ID NO: 72 - DNA, 20 bp, synthetic construct
  source 1..20: mol_type = other DNA
gcgaatcgcgcaatgttttc

SEQ ID NO: 73 - DNA, 20 bp, synthetic construct
  source 1..20: mol_type = other DNA
ggaaaacattgcgcgattcg

SEQ ID NO: 74 - DNA, 22 bp, synthetic construct
  source 1..22: mol_type = other DNA
cgctcatttcgtagctttggaa

SEQ ID NO: 75 - DNA, 20 bp, synthetic construct
  source 1..20: mol_type = other DNA
acgaaatgagcgcggtagag

SEQ ID NO: 76 - DNA, 22 bp, synthetic construct
  source 1..22: mol_type = other DNA
gattgccggttttcaggttctg

SEQ ID NO: 77 - DNA, 23 bp, synthetic construct
  source 1..23: mol_type = other DNA
ctgcatttaccgcacctacaaaa

SEQ ID NO: 78 - DNA, 22 bp, synthetic construct
  source 1..22: mol_type = other DNA
ccgcatatttgtccggttcgat

SEQ ID NO: 79 - DNA, 23 bp, synthetic construct
  source 1..23: mol_type = other DNA
ctgcatttaccgcacctacaaaa

SEQ ID NO: 80 - DNA, 23 bp, synthetic construct
  source 1..23: mol_type = other DNA
accaactcttcgtccgcatattt

SEQ ID NO: 81 - DNA, 22 bp, synthetic construct
  source 1..22: mol_type = other DNA
cctcaaccgtgtccgtaaagag

SEQ ID NO: 82 - DNA, 23 bp, synthetic construct
  source 1..23: mol_type = other DNA
cgcttcctgatgggttttattgc

SEQ ID NO: 83 - DNA, 22 bp, synthetic construct
  source 1..22: mol_type = other DNA
cctcaaccgtgtccgtaaagag

SEQ ID NO: 84 - DNA, 23 bp, synthetic construct
  source 1..23: mol_type = other DNA
cgcttcctgatgggttttattgc

SEQ ID NO: 85 - DNA, 23 bp, synthetic construct
  source 1..23: mol_type = other DNA
gcttgggcattacgcaataaaac

SEQ ID NO: 86 - DNA, 22 bp, synthetic construct
  source 1..22: mol_type = other DNA
tcagcgcaatccctatgacttc

SEQ ID NO: 87 - DNA, 23 bp, synthetic construct
  source 1..23: mol_type = other DNA
cccagaagctcgtgaagatcttg

SEQ ID NO: 88 - DNA, 20 bp, synthetic construct
  source 1..20: mol_type = other DNA
cgccaccttcgactgattca

SEQ ID NO: 89 - DNA, 23 bp, synthetic construct
  source 1..23: mol_type = other DNA
cccagaagctcgtgaagatcttg

SEQ ID NO: 90 - DNA, 20 bp, synthetic construct
  source 1..20: mol_type = other DNA
cgccaccttcgactgattca

SEQ ID NO: 91 - DNA, 24 bp, synthetic construct
  source 1..24: mol_type = other DNA
ccgtacaacaggtatcgttttcga

SEQ ID NO: 92 - DNA, 24 bp, synthetic construct
  source 1..24: mol_type = other DNA
gggagtagccttcgtaaattggaa

SEQ ID NO: 93 - DNA, 25 bp, synthetic construct
  source 1..25: mol_type = other DNA
acagttccaatttacgaaggctact

SEQ ID NO: 94 - DNA, 17 bp, synthetic construct
  source 1..17: mol_type = other DNA
cacggccagcgaggtta

SEQ ID NO: 95 - DNA, 17 bp, synthetic construct
  source 1..17: mol_type = other DNA
cgctggccgtgatctca

SEQ ID NO: 96 - DNA, 22 bp, synthetic construct
  source 1..22: mol_type = other DNA
ggctgttgtgttgaaagcattg

SEQ ID NO: 97 - DNA, 22 bp, synthetic construct
  source 1..22: mol_type = other DNA
ctggatggtcaagcttctcaca

SEQ ID NO: 98 - DNA, 25 bp, synthetic construct
  source 1..25: mol_type = other DNA
cttgatgtcacgaacgatttccttt

SEQ ID NO: 99 - DNA, 24 bp, synthetic construct
  source 1..24: mol_type = other DNA
ccgtacaacaggtatcgttttcga

SEQ ID NO: 100 - DNA, 24 bp, synthetic construct
  source 1..24: mol_type = other DNA
gggagtagccttcgtaaattggaa

SEQ ID NO: 101 - DNA, 25 bp, synthetic construct
  source 1..25: mol_type = other DNA
acagttccaatttacgaaggctact

SEQ ID NO: 102 - DNA, 21 bp, synthetic construct
  source 1..21: mol_type = other DNA
gccagcgaggttaagtctcat

SEQ ID NO: 103 - DNA, 21 bp, synthetic construct
  source 1..21: mol_type = other DNA
tgatctcacagcctggatggt

SEQ ID NO: 104 - DNA, 22 bp, synthetic construct
  source 1..22: mol_type = other DNA
gctgttgtgttgaaagcattgc

SEQ ID NO: 105 - DNA, 22 bp, synthetic construct
  source 1..22: mol_type = other DNA
ggatggtcaagcttctcacaga

SEQ ID NO: 106 - DNA, 25 bp, synthetic construct
  source 1..25: mol_type = other DNA
cttgatgtcacgaacgatttccttt

SEQ ID NO: 107 - DNA, 19 bp, synthetic construct
  source 1..19: mol_type = other DNA
agatttggacctgcgagcg

SEQ ID NO: 108 - DNA, 20 bp, synthetic construct
  source 1..20: mol_type = other DNA
gagcggctgtctccacaagt

SEQ ID NO: 109 - DNA, 18 bp, synthetic construct
  source 1..18: mol_type = other DNA
tagcaggtccgtcatttc

SEQ ID NO: 110 - DNA, 17 bp, synthetic construct
  source 1..17: mol_type = other DNA
acgacagacaaagactc

SEQ ID NO: 111 - DNA, 17 bp, synthetic construct
  source 1..17: mol_type = other DNA
cagctccgtcagagtct

SEQ ID NO: 112 - DNA, 16 bp, synthetic construct
  source 1..16: mol_type = other DNA
tccgacagaggcattc

SEQ ID NO: 113 - DNA, 16 bp, synthetic construct
  source 1..16: mol_type = other DNA
caggtccgtcatttcc

SEQ ID NO: 114 - DNA, 25 bp, synthetic construct
  source 1..25: mol_type = other DNA
tcctacacaacagggcctcgtaacg

SEQ ID NO: 115 - DNA, 29 bp, synthetic construct
  source 1..29: mol_type = other DNA
acgatggcatttccttctccaatgaaact

SEQ ID NO: 116 - DNA, 35 bp, synthetic construct
  source 1..35: mol_type = other DNA
accaacaaccaaactgatgatatcaatacgacaga

SEQ ID NO: 117 - DNA, 29 bp, synthetic construct
  source 1..29: mol_type = other DNA
caagatagcagagccctacaagaaactgt

SEQ ID NO: 118 - DNA, 29 bp, synthetic construct
  source 1..29: mol_type = other DNA
tcaagatagcagagccctacaagaaactg

SEQ ID NO: 119 - DNA, 29 bp, synthetic construct
  source 1..29: mol_type = other DNA
ctgctgcattcatccgagcatcttgaaga

SEQ ID NO: 120 - DNA, 29 bp, synthetic construct
  source 1..29: mol_type = other DNA
acgcttcttgcttatattcaaccggacgc

SEQ ID NO: 121 - DNA, 28 bp, synthetic construct
  source 1..28: mol_type = other DNA
tagcgagaagcgacatctttacgttcgc

SEQ ID NO: 122 - DNA, 29 bp, synthetic construct
  source 1..29: mol_type = other DNA
ttgagtgagctccccttctaaaagagctc

SEQ ID NO: 123 - DNA, 32 bp, synthetic construct
  source 1..32: mol_type = other DNA
ctgccgaaactaggaatatcgtagccatcact

SEQ ID NO: 124 - DNA, 30 bp, synthetic construct
  source 1..30: mol_type = other DNA
ctgccgaaactaggaatatcgtagccatca

SEQ ID NO: 125 - DNA, 27 bp, synthetic construct
  source 1..27: mol_type = other DNA
tctgacagggaatcgaactctcgcctt

SEQ ID NO: 126 - DNA, 31 bp, synthetic construct
  source 1..31: mol_type = other DNA
tcggatgtcaatgtaaccacagtcaatagct

SEQ ID NO: 127 - DNA, 32 bp, synthetic construct
  source 1..32: mol_type = other DNA
tccaaattgacagtgcaacaaactgttcttgt

SEQ ID NO: 128 - DNA, 32 bp, synthetic construct
  source 1..32: mol_type = other DNA
aaatgatccaaattgacagtgcaacaaactgt

SEQ ID NO: 129 - DNA, 34 bp, synthetic construct
  source 1..34: mol_type = other DNA
tctaccgtcccctaatgaaatactgaaaagatga

SEQ ID NO: 130 - DNA, 29 bp, synthetic construct
  source 1..29: mol_type = other DNA
ttgccctctgtagacaagaccatatccct

SEQ ID NO: 131 - DNA, 16 bp, synthetic construct
  source 1..16: mol_type = other DNA
cagccccttaagcttg

SEQ ID NO: 132 - DNA, 18 bp, synthetic construct
  source 1..18: mol_type = other DNA
ttgtttagcccaattgcc

SEQ ID NO: 133 - DNA, 18 bp, synthetic construct
  source 1..18: mol_type = other DNA
aacaccagatcaacctca

SEQ ID NO: 134 - DNA, 18 bp, synthetic construct
  source 1..18: mol_type = other DNA
caacacaagcaatcttct

SEQ ID NO: 135 - DNA, 19 bp, synthetic construct
  source 1..19: mol_type = other DNA
tagcggtttgctgtaatac

SEQ ID NO: 136 - DNA, 17 bp, synthetic construct
  source 1..17: mol_type = other DNA
actggcaggacggatat

SEQ ID NO: 137 - DNA, 15 bp, synthetic construct
  source 1..15: mol_type = other DNA
acgcaagcaagaacg

SEQ ID NO: 138 - DNA, 19 bp, synthetic construct
  source 1..19: mol_type = other DNA
cccatacagtccgatatgc

SEQ ID NO: 139 - DNA, 17 bp, synthetic construct
  source 1..17: mol_type = other DNA
cagcaaaccgctatctg

SEQ ID NO: 140 - DNA, 27 bp, synthetic construct
  source 1..27: mol_type = other DNA
acggttgtctatatccgtcctgccagt

SEQ ID NO: 141 - DNA, 24 bp, synthetic construct
  source 1..24: mol_type = other DNA
cctgccagtacgcaagcaagaacg

SEQ ID NO: 142 - DNA, 22 bp, synthetic construct
  source 1..22: mol_type = other DNA
tcgtttgccgccgccatatcca

SEQ ID NO: 143 - DNA, 20 bp, synthetic construct
  source 1..20: mol_type = other DNA
cagcaagccgcgctcgacaa

SEQ ID NO: 144 - DNA, 24 bp, synthetic construct
  source 1..24: mol_type = other DNA
catagacggacgcgccgcaatcac

SEQ ID NO: 145 - DNA, 22 bp, synthetic construct
  source 1..22: mol_type = other DNA
actgccttgccctgcttcagcg

SEQ ID NO: 146 - DNA, 25 bp, synthetic construct
  source 1..25: mol_type = other DNA
catggcggctgagtgcaatatcccc

SEQ ID NO: 147 - DNA, 19 bp, synthetic construct
  source 1..19: mol_type = other DNA
cccattgcctgcggcccat

SEQ ID NO: 148 - DNA, 21 bp, synthetic construct
  source 1..21: mol_type = other DNA
cctgcggcccatatcgaaccg

SEQ ID NO: 149 - DNA, 21 bp, synthetic construct
  source 1..21: mol_type = other DNA
cccaagcggtgtagcagccct

SEQ ID NO: 150 - DNA, 23 bp, synthetic construct
  source 1..23: mol_type = other DNA
ctgctacaccgcttgggcattac

SEQ ID NO: 151 - DNA, 23 bp, synthetic construct
  source 1..23: mol_type = other DNA
ccatcaggaagcgccgtggatac

SEQ ID NO: 152 - DNA, 20 bp, synthetic construct
  source 1..20: mol_type = other DNA
acttcgtcgtagtccttctc

SEQ ID NO: 153 - DNA, 27 bp, synthetic construct
  source 1..27: mol_type = other DNA
ctcctcgagaaggactacgacgaagtt

SEQ ID NO: 154 - DNA, 25 bp, synthetic construct
  source 1..25: mol_type = other DNA
ctgtgtgggaaacaccatcaccagc

SEQ ID NO: 155 - DNA, 25 bp, synthetic construct
  source 1..25: mol_type = other DNA
cccttccacacgccatcatgagact

SEQ ID NO: 156 - DNA, 25 bp, synthetic construct
  source 1..25: mol_type = other DNA
cacgctctgtgagaagcttgaccat

SEQ ID NO: 157 - DNA, 28 bp, synthetic construct
  source 1..28: mol_type = other DNA
ctgttgtgttgaaagcattgccacgctc

SEQ ID NO: 158 - DNA, 16 bp, synthetic construct
  source 1..16: mol_type = other DNA
ctgtgtgggaaacacc

SEQ ID NO: 159 - DNA, 17 bp, synthetic construct
  source 1..17: mol_type = other DNA
cccttccacacgccatc

SEQ ID NO: 160 - DNA, 19 bp, synthetic construct
  source 1..19: mol_type = other DNA
aagcttctcacagagcgtg

SEQ ID NO: 161 - DNA, 19 bp, synthetic construct
  source 1..19: mol_type = other DNA
ctgttgtgttgaaagcatt

SEQ ID NO: 162 - DNA, 23 bp, synthetic construct
  source 1..23: mol_type = other DNA
ttctgacctgaaggctctgcgcg

SEQ ID NO: 163 - DNA, 101 bp, Chlamydia trachomatis
  source 1..101: mol_type = genomic DNA
tctggagatgattcaggaagtgtctcttcctcagaatcagacaagaatgcctctgtcggaaatgacggacctgctatgaaagatatcctttctgccgtgcg

SEQ ID NO: 164 - DNA, 135 bp, Chlamydia trachomatis
  source 1..135: mol_type = genomic DNA
ctcaatctacagacgcctctacagatacaagcgatatcgatgacataaataccaacaaccaaactgatgatatcaatacgacagacaaagactctgacggagctggtggagtcaatggcgatatatccgaaacag

SEQ ID NO: 165 - DNA, 135 bp, Chlamydia trachomatis; identical to SEQ ID NO: 164
  source 1..135: mol_type = genomic DNA

SEQ ID NO: 166 - DNA, 101 bp, Chlamydia trachomatis; identical to SEQ ID NO: 163
  source 1..101: mol_type = genomic DNA

SEQ ID NO: 167 - DNA, 129 bp, Chlamydia trachomatis
  source 1..129: mol_type = genomic DNA
tggcgatatatccgaaacagaatcctcttctggagatgattcaggaagtgtctcttcctcagaatcagacaagaatgcctctgtcggaaatgacggacctgctatgaaagatatcctttctgccgtgcg

SEQ ID NO: 168 - DNA, 77 bp, Chlamydia trachomatis
  source 1..77: mol_type = genomic DNA
gcagcagcactcaattctctaagaggctcctcctacacaacagggcctcgtaacgagggtgtattcggccctggacc

SEQ ID NO: 169 - DNA, 98 bp, Chlamydia trachomatis
  source 1..98: mol_type = genomic DNA
ctcaagccgggcctagtagcgaagacgatggcatttccttctccaatgaaactcctggagctggtcctgcagctgctccatcaccaacgccatcctct

SEQ ID NO: 170 - DNA, 93 bp, Chlamydia trachomatis
  source 1..93: mol_type = genomic DNA
cgcctctacagatacaagcgatatcgatgacataaataccaacaaccaaactgatgatatcaatacgacagacaaagactctgacggagctgg

SEQ ID NO: 171 - DNA, 76 bp, Chlamydia trachomatis
  source 1..76: mol_type = genomic DNA
cagaacatgctcttcatcctaaagcacagtttcttgtagggctctgctatcttgaaatgggccatcctgacttagc

SEQ ID NO: 172 - DNA, 76 bp, Chlamydia trachomatis; identical to SEQ ID NO: 171
  source 1..76: mol_type = genomic DNA

SEQ ID NO: 173 - DNA, 89 bp, Chlamydia trachomatis
  source 1..89: mol_type = genomic DNA
gctttgcaagagccctataatgaacagtatcttcaagatgctcggatgaatgcagcagctttacgtaaacaacaccctaatcatcctag

SEQ ID NO: 174 - DNA, 88 bp, Chlamydia trachomatis
  source 1..88: mol_type = genomic DNA
actatattcatcacatgtgcgaagcttacgcttcttgcttatattcaaccggacgcttttatgagaaaaagcgaaaagcctcttctgc

SEQ ID NO: 175 - DNA, 88 bp, Chlamydia trachomatis
  source 1..88: mol_type = genomic DNA
caagaccttttttcgctgtttttgtcttttagcgaacgtaaagatgtcgcttctcgctatcatatcattcgagctcttttagaagggg

SEQ ID NO: 176 - DNA, 85 bp, Chlamydia trachomatis
  source 1..85: mol_type = genomic DNA
gatgtcgcttctcgctatcatatcattcgagctcttttagaaggggagctcactcaaagagagatagcagagaaatacggagtca

SEQ ID NO: 177 - DNA, 100 bp, Chlamydia trachomatis
  source 1..100: mol_type = genomic DNA
ggtgggattagtggtgataggactacttttagtgatggctacgatattcctagtttcggcagcttcgtttgtaaatcccatctatctagctattcctgct

SEQ ID NO: 178 - DNA, 100 bp, Chlamydia trachomatis; identical to SEQ ID NO: 177
  source 1..100: mol_type = genomic DNA

SEQ ID NO: 179 - DNA, 83 bp, Chlamydia trachomatis
  source 1..83: mol_type = genomic DNA
ggtggtgcgctttatgcaaagaatatcactctgacagggaatcgaactctcgcctttattaacaatatgtctggagactgcgg

SEQ ID NO: 180 - DNA, 103 bp, Chlamydia trachomatis
  source 1..103: mol_type = genomic DNA
gatagcaataccgctgctcatgccgggggagctattgactgtggttacattgacatccgaaataacggtcctatctattttctaaataactctgctgcctggg

SEQ ID NO: 181 - DNA, 105 bp, Mycoplasma genitalium
  source 1..105: mol_type = genomic DNA
ttttactcagttttatcccaggcttctttttagtacaacaagaacagtttgttgcactgtcaatttggatcattttgataacgctttttagtctttggtatgact

SEQ ID NO: 182 - DNA, 105 bp, Mycoplasma genitalium; identical to SEQ ID NO: 181
  source 1..105: mol_type = genomic DNA

SEQ ID NO: 183 - DNA, 86 bp, Mycoplasma genitalium
  source 1..86: mol_type = genomic DNA
ccaggtgtgaattgtatggtttgaaaatctaccgtcccctaatgaaatactgaaaagatgaaatcattgccctctgtagacaagac

SEQ ID NO: 184 - DNA, 118 bp, Mycoplasma genitalium
  source 1..118: mol_type = genomic DNA
ccgtcccctaatgaaatactgaaaagatgaaatcattgccctctgtagacaagaccatatcccttatgagattgatgaaactaacaagttacctatttataagcgcaatgaagtgagg

SEQ ID NO: 185 - DNA, 123 bp, Mycoplasma genitalium
  source 1..123: mol_type = genomic DNA
gctttaaaaccgcttgtcagttctagtttaatcttgattgttatcagccccttaagcttgtatttaaattcaagactaaatgcttttagtgtcttgtttaaccttttgtttagcccaattgcc

SEQ ID NO: 186 - DNA, 145 bp, Mycoplasma genitalium
  source 1..145: mol_type = genomic DNA
attgttatcagccccttaagcttgtatttaaattcaagactaaatgcttttagtgtcttgtttaaccttttgtttagcccaattgccttattttatttttgtgttagttgaattattttaccctttattggtgtttttggtcaag

SEQ ID NO: 187 - DNA, 118 bp, Mycoplasma genitalium
  source 1..118: mol_type = genomic DNA
gaagattgcttgtgttgttcatgtcaattacaacaccagatcaacctcattacgtgatcaaaagttagtagaacaatattgtcaaaaattaaatattccactggttgttcacactgtt

SEQ ID NO: 188 - DNA, 97 bp, Mycoplasma genitalium
  source 1..97: mol_type = genomic DNA
ggtggatctgattcaatgctaatgcttaaactttaccaaaagaagattgcttgtgttgttcatgtcaattacaacaccagatcaacctcattacgtg

SEQ ID NO: 189 - DNA, 98 bp, Neisseria gonorrhoeae
  source 1..98: mol_type = genomic DNA
ccatacagtccgatatgccaatagaagtaaaactgtattacgctaaatgtattacagcaaaccgctatctgttttcttatggacgtaaacccaaaggc

SEQ ID NO: 190 - DNA, 105 bp, Neisseria gonorrhoeae
  source 1..105: mol_type = genomic DNA
gcattgccgcaaccaaattaaaagaatttgaacaacggcaaaaagacacggttgtctatatccgtcctgccagtacgcaagcaagaacgctacgaagttatatcg

SEQ ID NO: 191 - DNA, 115 bp, Neisseria gonorrhoeae
  source 1..115: mol_type = genomic DNA
cattgccgcaaccaaattaaaagaatttgaacaacggcaaaaagacacggttgtctatatccgtcctgccagtacgcaagcaagaacgctacgaagttatatcgctcgtgatagc

SEQ ID NO: 192 - DNA, 129 bp, Neisseria gonorrhoeae
  source 1..129: mol_type = genomic DNA
gtagcaaatagtgatgtcgccgtattaacacccatacagtccgatatgccaatagaagtaaaactgtattacgctaaatgtattacagcaaaccgctatctgttttcttatggacgtaaacccaaaggc

SEQ ID NO: 193 - DNA, 117 bp, Neisseria gonorrhoeae
  source 1..117: mol_type = genomic DNA
gatgtcgccgtattaacacccatacagtccgatatgccaatagaagtaaaactgtattacgctaaatgtattacagcaaaccgctatctgttttcttatggacgtaaacccaaaggc

SEQ ID NO: 194 - DNA, 105 bp, Neisseria gonorrhoeae; identical to SEQ ID NO: 190
  source 1..105: mol_type = genomic DNA

SEQ ID NO: 195 - DNA, 81 bp, Neisseria gonorrhoeae
  source 1..81: mol_type = genomic DNA
cggcaaaaagacacggttgtctatatccgtcctgccagtacgcaagcaagaacgctacgaagttatatcgctcgtgatagc

SEQ ID NO: 196 - DNA, 78 bp, Neisseria gonorrhoeae
  source 1..78: mol_type = genomic DNA
ggcgcaacacaggattattttccgcgctctgctggatatggcggcggcaaacgagcctatcgacatcatcacgctgaa

SEQ ID NO: 197 - DNA, 91 bp, Neisseria gonorrhoeae
  source 1..91: mol_type = genomic DNA
cggtacgttgggattgtgaacgacaggtttgtcgagcgcggcttgctgaaggcttcggcggcgattgaaaaaatcgcggtttccaaagacg

SEQ ID NO: 198 - DNA, 83 bp, Neisseria gonorrhoeae
  source 1..83: mol_type = genomic DNA
ttgccggatggaaacctgattgtgattgcggcgcgtccgtctatgggtaaaacggttttggcggaaaacattgcgcgattcgc

SEQ ID NO: 199 - DNA, 68 bp, Neisseria gonorrhoeae
  source 1..68: mol_type = genomic DNA
ggaaaacattgcgcgattcgcgctgaagcagggcaaggcagttcatttccaaagctacgaaatgagcg

SEQ ID NO: 200 - DNA, 84 bp, Neisseria gonorrhoeae
  source 1..84: mol_type = genomic DNA
acgaaatgagcgcggtagagctggcaaggcgcggcatggcggctgagtgcaatatccccatgcagaacctgaaaaccggcaatc

SEQ ID NO: 201 - DNA, 77 bp, Neisseria gonorrhoeae
  source 1..77: mol_type = genomic DNA
ctgcatttaccgcacctacaaaaaatacggcggcagcccattgcctgcggcccatatcgaaccggacaaatatgcgg

SEQ ID NO: 202 - DNA, 90 bp, Neisseria gonorrhoeae
  source 1..90: mol_type = genomic DNA
ctgcatttaccgcacctacaaaaaatacggcggcagcccattgcctgcggcccatatcgaaccggacaaatatgcggacgaagagttggt

SEQ ID NO: 203 - DNA, 73 bp, Neisseria gonorrhoeae
  source 1..73: mol_type = genomic DNA
cctcaaccgtgtccgtaaagagcagggctgctacaccgcttgggcattacgcaataaaacccatcaggaagcg

SEQ ID NO: 204 - DNA, 73 bp, Neisseria gonorrhoeae; identical to SEQ ID NO: 203
  source 1..73: mol_type = genomic DNA

SEQ ID NO: 205 - DNA, 82 bp, Neisseria gonorrhoeae
  source 1..82: mol_type = genomic DNA
gcttgggcattacgcaataaaacccatcaggaagcgccgtggatacagacccggcagggcgaagtcatagggattgcgctga

SEQ ID NO: 206 - DNA, 77 bp, unidentified
  source 1..77: mol_type = unassigned DNA
cccagaagctcgtgaagatcttgccctcctcgagaaggactacgacgaagttgctgctgaatcagtcgaaggtggcg

SEQ ID NO: 207 - DNA, 77 bp, unidentified; identical to SEQ ID NO: 206
  source 1..77: mol_type = unassigned DNA

SEQ ID NO: 208 - DNA, 74 bp, unidentified
  source 1..74: mol_type = unassigned DNA
ccgtacaacaggtatcgttttcgatgctggtgatggtgtttcccacacagttccaatttacgaaggctactccc

SEQ ID NO: 209 - DNA, 67 bp, unidentified
  source 1..67: mol_type = unassigned DNA
acagttccaatttacgaaggctactcccttccacacgccatcatgagacttaacctcgctggccgtg

SEQ ID NO: 210 - DNA, 73 bp, unidentified
  source 1..73: mol_type = unassigned DNA
cgctggccgtgatctcacagcctggatggtcaagcttctcacagagcgtggcaatgctttcaacacaacagcc

SEQ ID NO: 211 - DNA, 79 bp, unidentified
  source 1..79: mol_type = unassigned DNA
ctggatggtcaagcttctcacagagcgtggcaatgctttcaacacaacagccgaaaaggaaatcgttcgtgacatcaag

SEQ ID NO: 212 - DNA, 74 bp, unidentified; identical to SEQ ID NO: 208
  source 1..74: mol_type = unassigned DNA

SEQ ID NO: 213 - DNA, 63 bp, unidentified
  source 1..63: mol_type = unassigned DNA
acagttccaatttacgaaggctactcccttccacacgccatcatgagacttaacctcgctggc

SEQ ID NO: 214 - DNA, 63 bp, unidentified
  source 1..63: mol_type = unassigned DNA
tgatctcacagcctggatggtcaagcttctcacagagcgtggcaatgctttcaacacaacagc

SEQ ID NO: 215 - DNA, 77 bp, unidentified
  source 1..77: mol_type = unassigned DNA
ggatggtcaagcttctcacagagcgtggcaatgctttcaacacaacagccgaaaaggaaatcgttcgtgacatcaag

SEQ ID NO: 216 - DNA, 65 bp, Homo sapiens
  source 1..65: mol_type = genomic DNA
agatttggacctgcgagcgggttctgacctgaaggctctgcgcggacttgtggagacagccgctc

SEQ ID NO: 217 - DNA, 151 bp, Chlamydia trachomatis
  source 1..151: mol_type = genomic DNA
cgatatatccgaaacagaatcctcttctggagatgattcaggaagtgtctcttcctcagaatcagacaagaatgcctctgtcggaaatgacggacctgctatgaaagatatcctttctgccgtgcgtaaacacctagacgtcgtttaccct

SEQ ID NO: 218 - DNA, 185 bp, Chlamydia trachomatis
  source 1..185: mol_type = genomic DNA
aacacgactaacaccacaccaacaactcaatctacagacgcctctacagatacaagcgatatcgataacataaataccaacaaccaaactgatgatatcaatacgacagacaaagactctgacggagctggtggagtcaatggcgatatatccgaaacagaatcctcttctggagatgattcagg

SEQ ID NO: 219 - DNA, 185 bp, Chlamydia trachomatis; identical to SEQ ID NO: 218
  source 1..185: mol_type = genomic DNA

SEQ ID NO: 220 - DNA, 151 bp, Chlamydia trachomatis; identical to SEQ ID NO: 217
  source 1..151: mol_type = genomic DNA

SEQ ID NO: 221 - DNA, 179 bp, Chlamydia trachomatis
  source 1..179: mol_type = genomic DNA
actctgacggagctggtggagtcaatggcgatatatccgaaacagaatcctcttctggagatgattcaggaagtgtctcttcctcagaatcagacaagaatgcctctgtcggaaatgacggacctgctatgaaagatatcctttctgccgtgcgtaaacacctagacgtcgtttaccct

SEQ ID NO: 222 - DNA, 127 bp, Chlamydia trachomatis
  source 1..127: mol_type = genomic DNA
cccagaaaatacgagtggtggtgcagcagcagcactcaattctctaagaggctcctcctacacaacagggcctcgtaacgagggtgtattcggccctggaccggaaggactaccagacatgtctctt

SEQ ID NO: 223 - DNA, 148 bp, Chlamydia trachomatis
  source 1..148: mol_type = genomic DNA
gactttaataaaatcaactggggaactcaagccgggcctagtagcgaagacgatggcatttccttctccaatgaaactcctggagctggtcctgcagctgctccatcaccaacgccatcctctattcctgtcatcaatgtcaatgtca

SEQ ID NO: 224 - DNA, 143 bp, Chlamydia trachomatis
  source 1..143: mol_type = genomic DNA
ccacaccaacaactcaatctacagacgcctctacagatacaagcgatatcgataacataaataccaacaaccaaactgatgatatcaatacgacagacaaagactctgacggagctggtggagtcaatggcgatatatccgaa

SEQ ID NO: 225 - DNA, 126 bp, Chlamydia trachomatis
  source 1..126: mol_type = genomic DNA
tttggaatgataacacatcatttcccagaacatgctcttcatcctaaagcacagtttcttgtagggctctgctatcttgaaatgggccatcctgacttagcggataaagcgctaactcaatatcaa

SEQ ID NO: 226 - DNA, 126 bp, Chlamydia trachomatis; identical to SEQ ID NO: 225
  source 1..126: mol_type = genomic DNA

SEQ ID NO: 227 - DNA, 139 bp, Chlamydia trachomatis
  source 1..139: mol_type = genomic DNA
tattgcaaaaatccattgcttacaagctttgcaagagccctataatgaacagtatcttcaagatgctcggatgaatgcagcagctttacgtaaacaacaccctaatcatcctagcaatacagaagtagagaactatatt

SEQ ID NO: 228 - DNA, 138 bp, Chlamydia trachomatis
  source 1..138: mol_type = genomic DNA
catcctagcaatacagaagtagagaactatattcatcacatgtgcgaagcttacgcttcttgcttatattcaaccggacgcttttatgagaaaaagcgaaaagcctcttctgcaaaaatttattactcaatagctcta

SEQ ID NO: 229 - DNA, 138 bp, Chlamydia trachomatis
  source 1..138: mol_type = genomic DNA
taaaatgcaaaaagaaaagtttttacaagaccttttttcgctgtttttgtcttttagcgaacgtaaagatgtcgcttctcgctatcatatcattcgagctcttttagaaggggagctcactcaaagagagatagcaga

SEQ ID NO: 230 - DNA, 135 bp, Chlamydia trachomatis
  source 1..135: mol_type = genomic DNA
gtttttgtcttttggcgaacgtaaagatgtcgcttctcgctatcatatcattcgagctcttttagaaggggagctcactcaaagagagatagcagagaaatacggagtcagtatcgcacaaattaccagaggatc

SEQ ID NO: 231 - DNA, 150 bp, Chlamydia trachomatis
  source 1..150: mol_type = genomic DNA
cgcagatattcacgcatcccacaatagcaggaatagctagatagatgggatttacaaacgaagctgccgaaactaggaatatcgtagccatcactaaaagtagtcctatcaccactaatcccaccttaaatgcagtggaagatagaagat

SEQ ID NO: 232 - DNA, 150 bp, Chlamydia trachomatis; identical to SEQ ID NO: 231
  source 1..150: mol_type = genomic DNA

SEQ ID NO: 233 - DNA, 133 bp, Chlamydia trachomatis
  source 1..133: mol_type = genomic DNA
ttgagtgttagcggcgatagctcccccgcagtctccagacatattgttaataaaggcgagagttcgattccctgtcagagtgatattctttgcataaagcgcaccacccctatttttttcgcttttatttcct

SEQ ID NO: 234 - DNA, 153 bp, Chlamydia trachomatis
  source 1..153: mol_type = genomic DNA
agtttcgataaattaaaggctgctccccaggcagcagagttatttagaaaatagataggaccgttatttcggatgtcaatgtaaccacagtcaatagctcccccggcatgagcagcggtattgctatcgaaatagataggacctttattgttt

SEQ ID NO: 235 - DNA, 155 bp, Mycoplasma genitalium
  source 1..155: mol_type = genomic DNA
gttaagttcaataaacaaaactttcagtcataccaaagactaaaaagcgttatcaaaatgatccaaattgacagtgcaacaaactgttcttgttgtactaaaaagaagcctgggataaaactgagtaaaacaaaaaaataaaacagctttgtttg

SEQ ID NO: 236 - DNA, 155 bp, Mycoplasma genitalium; identical to SEQ ID NO: 235
  source 1..155: mol_type = genomic DNA

SEQ ID NO: 237 - DNA, 136 bp, Mycoplasma genitalium
  source 1..136: mol_type = genomic DNA
cgtgctgtttactatgggattaaaaccaggtgtgaattgtatggtttgaaaatctaccgtcccctaatgaaatactgaaaagatgaaatcattgccctctgtagacaagaccatatcccttatgagattgatgaaa

SEQ ID NO: 238 - DNA, 168 bp, Mycoplasma genitalium
  source 1..168: mol_type = genomic DNA
gtgaattgtatggtttgaaaatctaccgtcccctaatgaaatactgaaaagatgaaatcattgccctctgtagacaagaccatatcccttatgagattgatgaaactaacaagttacctatttataagcgcaatgaagtgaggttagagatagaaaaatggtctaaaa

SEQ ID NO: 239 - DNA, 173 bp, Mycoplasma genitalium
  source 1..173: mol_type = genomic DNA
ttcaactaacacaaaaataaaataaggcaattgggctaaacaaaaggttaaacaagacactaaaagcatttagtcttgaatttaaatacaagcttaaggggctgataacaatcaagattaaactagaactgacaagcggttttaaagcttttaataactttagtttgttgaca

SEQ ID NO: 240 - DNA, 195 bp, Mycoplasma genitalium
  source 1..195: mol_type = genomic DNA
agtgggagataaataccaaaactagcttgaccaaaaacaccaataaagggtaaaataattcaactaacacaaaaataaaataaggcaattgggctaaacaaaaggttaaacaagacactaaaagcatttagtcttgaatttaaatacaagcttaaggggctgataacaatcaagattaaactagaactgacaagc

SEQ ID NO: 241 - DNA, 168 bp, Mycoplasma genitalium
  source 1..168: mol_type = genomic DNA
tgctaatgcttaaactttaccaaaagaagattgcttgtgttgttcatgtcaattacaacaccagatcaacctcattacgtgatcaaaagttagtagaacaatactgtcaaaaattaaatattccactggttgttcacactgttgatcctgatctagtttgaaagaaga

SEQ ID NO: 242 - DNA, 147 bp, Mycoplasma genitalium
  source 1..147: mol_type = genomic DNA
aaaacaatatatagcaggggtttcaggtggatctgattcaatgctaatgcttaaactttaccaaaagaagattgcttgtgttgttcatgtcaattacaacaccagatcaacctcattacgtgatcaaaagttagtagaacaatactg

SEQ ID NO: 243 - DNA, 148 bp, Neisseria gonorrhoeae
  source 1..148: mol_type = genomic DNA
aatagtgatgtcgccgtattaacacccatacagtccgatatgccaatagaagtaaaactgtattacgctaaatgtattacagcaaaccgctatctgttttcttatggacgtaaacccaaaggcgaaaaattaaaaagcattatgttac

SEQ ID NO: 244 - DNA, 155 bp, Neisseria gonorrhoeae
  source 1..155: mol_type = genomic DNA
gatgaaatttttactattcaaaacggcattgccgcaaccaaattaaaagaatttgaacaacggcaaaaagacacggttgtctatatccgtcctgccagtacgcaagcaagaacgctacgaagttatatcgctcgtgatagcgttgatgaaaaaca

SEQ ID NO: 245 - DNA, 165 bp, Neisseria gonorrhoeae
  source 1..165: mol_type = genomic DNA
atgaaatttttactattcaaaacggcattgccgcaaccaaattaaaagaatttgaacaacggcaaaaagacacggttgtctatatccgtcctgccagtacgcaagcaagaacgctacgaagttatatcgctcgtgatagcgttgatgaaaaacatatttttccat

SEQ ID NO: 246 - DNA, 179 bp, Neisseria gonorrhoeae
  source 1..179: mol_type = genomic DNA
tagctatgtttcaacttgtgaatttgtagcaaatagtgatgtcgccgtattaacacccatacagtccgatatgccaatagaagtaaaactgtattacgctaaatgtattacagcaaaccgctatctgttttcttatggacgtaaacccaaaggcgaaaaattaaaaagcattatgttac

SEQ ID NO: 247 - DNA, 179 bp, Neisseria gonorrhoeae; identical to SEQ ID NO: 246
  source 1..179: mol_type = genomic DNA

SEQ ID NO: 248 - DNA, 155 bp, Neisseria gonorrhoeae; identical to SEQ ID NO: 244
  source 1..155: mol_type = genomic DNA

SEQ ID NO: 249 - DNA, 131 bp, Neisseria gonorrhoeae
  source 1..131: mol_type = genomic DNA
aaccaaattaaaagaatttgaacaacggcaaaaagacacggttgtctatatccgtcctgccagtacgcaagcaagaacgctacgaagttatatcgctcgtgatagcgttgatgaaaaacatatttttccat

SEQ ID NO: 250 - DNA, 128 bp, Neisseria gonorrhoeae
  source 1..128: mol_type = genomic DNA
ctcgccgcgcgcttccagcttgtcgttcagcgtgatgatgtcgataggctcgtttgccgccgccatatccagcagagcgcggaaaataatcctgtgttgcgcctggtaaaacttttcaggggtcagga

SEQ ID NO: 251 - DNA, 141 bp, Neisseria gonorrhoeae
  source 1..141: mol_type = genomic DNA
gacagcttttctgcgaccgtcccgccgtctttggaaaccgcgattttttcaatcgccgccgaagccttcagcaagccgcgctcgacaaacctgtcgttcacaatcccaacgtaccggctgatattcttcgcgcttggggtg

SEQ ID NO: 252 - DNA, 133 bp, Neisseria gonorrhoeae
  source 1..133: mol_type = genomic DNA
atgaactgccttgccctgcttcagcgcgaatcgcgcaatgttttccgccaaaaccgttttacccatagacggacgcgccgcaatcacaatcaggtttccatccggcaaaccgccggtcatcccgtcaagcttc

SEQ ID NO: 253 - DNA, 118 bp, Neisseria gonorrhoeae
  source 1..118: mol_type = genomic DNA
atgccgcgccttgccagctctaccgcgctcatttcgtagctttggaaatgaactgccttgccctgcttcagcgcgaatcgcgcaatgttttccgccaaaaccgttttacccatagacg

SEQ ID NO: 254 - DNA, 134 bp, Neisseria gonorrhoeae
  source 1..134: mol_type = genomic DNA
atattggcgtaatcgctttgcgtcagattgccggttttcaggttctgcatggggatattgcactcagccgccatgccgcgccttgccagctctaccgcgctcatttcgtagctttggaaatgaactgccttgcc

SEQ ID NO: 255 - DNA, 127 bp, Neisseria gonorrhoeae
  source 1..127: mol_type = genomic DNA
cggttgaggacaaccaactcttcgtccgcatatttgtccggttcgatatgggccgcaggcaatgggctgccgccgtattttttgtaggtgcggtaaatgcaggggactaccggaccgtgctgccagt

SEQ ID NO: 256 - DNA, 140 bp, Neisseria gonorrhoeae
  source 1..140: mol_type = genomic DNA
ctctttacggacacggttgaggacaaccaactcttcgtccgcatatttgtccggttcgatatgggccgcaggcaatgggctgccgccgtattttttgtaggtgcggtaaatgcaggggactaccggaccgtgctgccagt

SEQ ID NO: 257 - DNA, 123 bp, Neisseria gonorrhoeae
  source 1..123: mol_type = genomic DNA
cgccctgccgggtctgtatccacggcgcttcctgatgggttttattgcgtaatgcccaagcggtgtagcagccctgctctttacggacacggttgaggacaaccaactcttcgtccgcatatt

SEQ ID NO: 258 - DNA, 123 bp, Neisseria gonorrhoeae; identical to SEQ ID NO: 257
  source 1..123: mol_type = genomic DNA

SEQ ID NO: 259 - DNA, 132 bp, Neisseria gonorrhoeae
  source 1..132: mol_type = genomic DNA
agcgcatggcggaaatattcccccatcagcgcaatccctatgacttcgccctgccgggtctgtatccacggcgcttcctgatgggttttattgcgtaatgcccaagcggtgtagcagccctgctctttacgg

SEQ ID NO: 260 - DNA, 127 bp, unidentified
  source 1..127: mol_type = unassigned DNA
gtgaaggtatggaagaagctgaattcccagaagctcgtgaagatcttgccctcctcgagaaggactacgacgaagttgctgctgaatcagtcgaaggtggcgacgaagaagaagatggtggcgaaat

SEQ ID NO: 261 - DNA, 127 bp, unidentified; identical to SEQ ID NO: 260
  source 1..127: mol_type = unassigned DNA

SEQ ID NO: 262 - DNA, 124 bp, unidentified
  source 1..124: mol_type = unassigned DNA
ctgttctttccctctactcctctggccgtacaacaggtatcgttttcgatgctggtgatggtgtttcccacacagttccaatttacgaaggctactcccttccacacgccatcatgagacttaa

SEQ ID NO: 263 - DNA, 117 bp, unidentified
  source 1..117: mol_type = unassigned DNA
cgatgctggtgatggtgtttcccacacagttccaatttacgaaggctactcccttccacacgccatcatgagacttaacctcgctggccgtgatctcacagcctggatggtcaagct

SEQ ID NO: 264 - DNA, 123 bp, unidentified
  source 1..123: mol_type = unassigned DNA
cacacgccatcatgagacttaacctcgctggccgtgatctcacagcctggatggtcaagcttctcacagagcgtggcaatgctttcaacacaacagccgaaaaggaaatcgttcgtgacatca

SEQ ID NO: 265 - DNA, 129 bp, unidentified
  source 1..129: mol_type = unassigned DNA
acctcgctggccgtgatctcacagcctggatggtcaagcttctcacagagcgtggcaatgctttcaacacaacagccgaaaaggaaatcgttcgtgacatcaaggagaagctttgctatgtcgccctcg

SEQ ID NO: 266 - DNA, 124 bp, unidentified; identical to SEQ ID NO: 262
  source 1..124: mol_type = unassigned DNA

SEQ ID NO: 267 - DNA, 113 bp, unidentified
  source 1..113: mol_type = unassigned DNA
cgatgctggtgatggtgtttcccacacagttccaatttacgaaggctactcccttccacacgccatcatgagacttaacctcgctggccgtgatctcacagcctggatggtca

SEQ ID NO: 268 - DNA, 113 bp, unidentified
  source 1..113: mol_type = unassigned DNA
tcatgagacttaacctcgctggccgtgatctcacagcctggatggtcaagcttctcacagagcgtggcaatgctttcaacacaacagccgaaaaggaaatcgttcgtgacatc

SEQ ID NO: 269 - DNA, 127 bp, unidentified
  source 1..127: mol_type = unassigned DNA
ctcgctggccgtgatctcacagcctggatggtcaagcttctcacagagcgtggcaatgctttcaacacaacagccgaaaaggaaatcgttcgtgacatcaaggagaagctttgctatgtcgccctcg

SEQ ID NO: 270 - DNA, 115 bp, Homo sapiens
  source 1..115: mol_type = genomic DNA
gggacttcagcatggcggtgtttgcagatttggacctgcgagcgggttctgacctgaaggctctgcgcggacttgtggagacagccgctcaccttggctattcagttgttgctat

SEQ ID NO: 271 - DNA, 1237 bp, synthetic construct
  source 1..1237: mol_type = other DNA
ggccgtcaaggccaacacgactaacaccacaccaacaactcaatctacagacgcctctacagatacaagcgatatcgataacataaataccaacaaccaaactgatgatatcaatacgacagacaaagactctgacggagctggtggagtcaatggcgatatatccgaaacagaatcctcttctggagatgattcaggaagtgtctcttcctcagaatcagacaagaatgcctctgtcggaaatgacggacctgctatgaaagatatcctttctgccgtgcgtaaacacctagacgtcgtttaccctaaaacaatatatagcaggggtttcaggtggatctgattcaatgctaatgcttaaactttaccaaaagaagattgcttgtgttgttcatgtcaattacaacaccagatcaacctcattacgtgatcaaaagttagtagaacaatactgtcaaaaattaaatattccactggttgttcacactgttgatcctgatctagtttgaaagaagaatgaaatttttactattcaaaacggcattgccgcaaccaaattaaaagaatttgaacaacggcaaaaagacacggttgtctatatccgtcctgccagtacgcaagcaagaacgctacgaagttatatcgctcgtgatagcgttgatgaaaaacatatttttccatctgttctttccctctactcctctggccgtacaacaggtatcgttttcgatgctggtgatggtgtttcccacacagttccaatttacgaaggctactcccttccacacgccatcatgagacttaacctcgctggccgtgatctcacagcctggatggtcaagcttctcacagagcgtggcaatgctttcaacacaacagccgaaaaggaaatcgttcgtgacatcaaggagaagctttgctatgtcgccctcgatggcggtgtttgcagatttggacctgcgagcgggttctgacctgaaggctctgcgcggacttgtggagacagccgctcaccgtgagttgcacagatctaagcgtggaagtggctatatagagcagatatattatatgacgtaccagaggatcacctactaaaagacttttcgagaatctacacctactagcaagggtgcggagggaagctcatcagtggggccacgagctgagtgcgtcctgtcactccactcccatgtcccttgggaaggtctgagactagggacgcgtggcctcatgggcc

(19) United States
(12) Patent Application Publication
(10) Pub. No.: US 2024/0254568 A1
(43) Pub. Date: Aug. 1, 2024

(54) COMPOSITIONS, KITS, AND METHODS FOR DETECTING STI PATHOGEN SEQUENCES

(71) Applicant: Life Technologies Corporation, Carlsbad, CA (US)

(72) Inventors: Yulei Shang, San Francisco, CA (US); Kelly Li, Redwood City, CA (US); Pius Brzoska, Woodside, CA (US); Ioanna Pagani, Oakland, CA (US); Stephen Williams, San Carlos, CA (US); Chunling Wang, Redwood City, CA (US)

(21) Appl. No.: 18/422,468

(22) Filed: Jan. 25, 2024

Related U.S. Application Data
(60) Provisional application No. 63/481,986, filed on Jan. 27, 2023.

(51) Int. Cl. C12Q 1/689; C12Q 1/6893

(52) U.S. Cl. CPC C12Q 1/689; C12Q 1/6893; C12Q 2600/16; C12Q 2600/166

(57) ABSTRACT

Compositions, assays, methods, diagnostic methods, kits, and diagnostic kits are disclosed for the specific and differential detection of sexually transmitted infection (STI) pathogens in biological samples obtained from a human or non-human animal. The compositions, assays, methods, diagnostic methods, kits, and diagnostic kits provide materials and method steps for differential detection of any one of Chlamydia trachomatis (CT), Mycoplasma genitalium (MG), Neisseria gonorrhoeae (NG); and Trichomonas vaginalis (TV) in a single multiplex assay.

### CROSS-REFERENCE TO RELATED APPLICATIONS

This application claims priority to and the benefit of U.S. Provisional Patent Application No. 63/481,986, filed Jan. 27, 2023, the disclosure of which is incorporated herein by reference in its entirety.

### FIELD

The present disclosure relates to compositions, methods, systems and kits for specific detection, diagnosis and differentiation of pathogens involved in sexually transmitted infections (STIs).

### SEQUENCE LISTING

This application contains a Sequence Listing XML, which has been submitted electronically as an XML formatted sequence listing with a file name “087333_008492_SL.xml” and a creation date of Jan. 22, 2024, and having a size of 252,367 bites. The sequence listing submitted electronically is part of the specification and is herein incorporated by reference in its entirety.

### BACKGROUND

Differential detection of specific STI pathogens allows accurate diagnosis so that appropriate treatment and infection control measures can be provided in a timely manner. Current methods, systems, and kits for detection of specific STI pathogens suffer from lack of detection of the available strains of STI pathogens. The reason behind these shortcomings is an incomplete pathogen strain coverage of the available methods, systems, and kits. This can lead to misdiagnosis of the disease and mistakes in disease treatment. Accordingly, there are a number of disadvantages with current methods, systems, compositions, and kits for detecting STI pathogens, which can be addressed, and the methods, systems, compositions, and kits of the present disclosure address and overcome at least some of the foregoing problems in the art.

### SUMMARY

Provided are compositions for amplifying target sequences from sexually transmitted infection (STI) pathogen genomes at greater than 99% strain coverage for each pathogen. The composition comprise (a) a first set of forward primers and reverse primers for amplifying one or more target sequences present in target regions of the Chlamydia trachomatis (CT) genome; (b) a second set of forward primers and reverse primers for amplifying one or more target sequences present in target regions of the Mycoplasma genitalium (MG) genome; (c) a third set of forward primers and reverse primers for amplifying one or more target sequences present in target regions of the Neisseria gonorrhoeae (NG) genome; and (d) a fourth set of forward primers and reverse primers for amplifying one or more target sequences present in target regions of the Trichomonas vaginalis (TV) genome.

The first set of forward primers and reverse primers can comprise a first forward primer and a first reverse primer configured to amplify a first target sequence present in a first target region of the CT genome, wherein the first target sequence includes at least 10 contiguous nucleotides of the first target region of the CT genome. The first set of forward primers and reverse primers can comprise a second forward primer and a second reverse primer configured to amplify a second target sequence present in a second target region of the CT genome, wherein the second target sequence includes at least 10 contiguous nucleotides of the second target region of the CT genome. The first set of forward primers and reverse primers can comprise a third forward primer and a third reverse primer configured to amplify a third target sequence present in a third target region of the CT genome, wherein the third target sequence includes at least 10 contiguous nucleotides of the third target region of the CT genome. The first set of forward primers and reverse primers can comprise a fourth forward primer and a fourth reverse primer configured to amplify a fourth target sequence present in a fourth target region of the CT genome, wherein the fourth target sequence includes at least 10 contiguous nucleotides of the fourth target region of the CT genome. The first set of forward primers and reverse primers can comprise a fifth forward primer and a fifth reverse primer configured to amplify a fifth target sequence present in a fifth target region of the CT genome, wherein the fifth target sequence includes at least 10 contiguous nucleotides of the fifth target region of the CT genome.

The second set of forward primers and reverse primers can comprise a first forward primer and a first reverse primer configured to amplify a first target sequence present in a first target region of the MG genome, wherein the first target sequence includes at least 10 contiguous nucleotides of the first target region of the MG genome. The second set of forward primers and reverse primers can comprise a second forward primer and a second reverse primer configured to amplify a second target sequence present in a second target region of the MG genome, wherein the second target sequence includes at least 10 contiguous nucleotides of the second target region of the MG genome.

The third set of forward primers and reverse primers can comprise a first forward primer and a first reverse primer configured to amplify a first target sequence present in a first target region of the NG genome, wherein the first target sequence includes at least 10 contiguous nucleotides of the first target region of the NG genome. The third set of forward primers and reverse primers can comprise a second forward primer and a second reverse primer configured to amplify a second target sequence present in a second target region of the NG genome, wherein the second target sequence includes at least 10 contiguous nucleotides of the second target region of the NG genome. The third set of forward primers and reverse primers can comprise a third forward primer and a third reverse primer configured to amplify a third target sequence present in a third target region of the NG genome, wherein the third target sequence includes at least 10 contiguous nucleotides of the third target region of the NG genome. The third set of forward primers and reverse primers can comprise a fourth forward primer and fourth reverse primer configured to amplify a fourth target sequence present in a fourth target region of the NG genome, wherein the fourth target sequence includes at least 10 contiguous nucleotides of the fourth target region of the NG genome.

The fourth set of forward primers and reverse primers can comprise a first forward primer and a first reverse primer configured to amplify a first target sequence present in a first target region of the TV genome, wherein the first target sequence includes at least 10 contiguous nucleotides of the first target region of the TV genome. The fourth set of forward primers and reverse primers can comprise a second forward primer and a second reverse primer configured to amplify a second target sequence present in a second target region of the TV genome, wherein the second target sequence includes at least 10 contiguous nucleotides of the second target region of the TV genome.

The composition can comprise a set of forward primers and reverse primers comprising a first forward primer and a first reverse primer configured to amplify a first target sequence present in a first target region of a control gene, wherein the first target sequence includes at least 10 contiguous nucleotides of the control gene.

The target regions amplified by the first set of forward primers and reverse primers can comprise the CT genome, such as the regions within the tarP, BamD, Trp operon repressor, cysteine-rich outer membrane protein (CRPA), and/or the polymorphic outer membrane protein repeat-containing protein gene regions of CT. The target regions of the CT genome can be selected from SEQ ID NOs: 163-180.

The target regions amplified by the second set of forward primers and reverse primers can comprise the MG genome, such as the regions within the ComEC/Rec2-related protein gene and/or the tRNA(Ile)-lysidine synthetase gene regions of MG. The target regions of the MG genome can be selected from SEQ ID NOs: 181-189.

The target regions amplified by the third set of forward primers and reverse primers can comprise the NG genome, such as the regions within the NGO0357, the NGO_0357 hypothetical protein, the NGO_1110 phage protein, and/or the NGO_1117 phage protein gene regions of NG. The target regions of the NG genome can be selected from SEQ ID NOs: 190-205.

The target regions amplified by the fourth set of forward primers and reverse primers can comprise the TV genome, such as the regions within the alpha-tubulin-1 (α-TUB1) and/or the actin gene regions of TV. The target regions of the TV genome can be selected from SEQ ID NOS: 206-215.

The composition can further comprise a fifth set of forward primers and reverse primers for amplifying a first target sequence present in a first target region of a control gene. The control gene can comprise human RNaseP gene. The target region of the control gene can comprises SEQ ID NO: 216.

The compositions can further comprise a nucleic acid sample, a polymerase, a buffer, and nucleotides.

The compositions can further comprise a first probe, a second probe, a third probe, a fourth probe, and/or a fifth probe. Each of the first probe, the second probe, the third probe, the fourth probe, and/or the fifth probe can comprise at least one label.

The label for the first probe can comprise a first label of the composition, the label for the second probe can comprise a second label of the composition, the label for the third probe can comprise a third label of the composition, the label for the fourth probe can comprise a fourth label of the composition, and/or the label for the fifth probe can comprise a fifth label of the composition. The first label, the second label, the third label, the fourth label, and or the fifth label can comprise a fluorescent or other detectable label.

The probes can comprise a quencher or a minor groove-binder (MGB). The fluorescent label can be selected from FAM™, VIC™, ABY™, JUN™; FITC; 6-carboxy-4′,5′-dichloro-2′,7′-dimethoxy fluorescein (JOE™); 6-carboxy-1,4-dichloro-2′,7′-dichloro fluorescein (TET™); 6-carboxy-1,4-dichloro-2′,4′,5′,7′-tetra-chlorofluorescein (HEX™); Alexa Fluor™ fluorophores (e.g., Alexa 647), NED™, and BODIPY™ fluorophores. The quencher can be selected from QSY® and BHQ™ dyes.

The compositions can comprise the first set of forward primers and reverse primers selected from SEQ ID NOs: 1-36 for amplifying the one or more target sequences present in the target regions of the CT genome.

The compositions can comprise the second set of forward primers and reverse primers selected from SEQ ID NOs: 37-52 for amplifying the one or more target sequences present in the target regions of the MG genome.

The compositions can comprise the third set of forward primers and reverse primers selected from SEQ ID NOs: 53-86 for amplifying the one or more target sequences present in the target regions of the NG genome.

The compositions can comprise the fourth set of forward primers and reverse primers selected from SEQ ID NOs: 87-106 for amplifying the one or more target sequences present in the target regions of the TV genome.

The compositions can comprise the fifth set of forward primers and reverse primers comprising SEQ ID NOs: 107 and 108 for amplifying the target sequence present in the target region of a control gene.

The compositions can comprise the first probe, the second probe, the third probe, the fourth probe, and/or the fifth probe for detecting the one or more target sequences present in the target regions of the CT, MG, NG, TV genomes, and/or the control gene. The compositions can comprise the probes selected from SEQ ID NOs: 109-162.

The compositions can comprise the first probe labeled with FAM™ fluorescent label, the second probe labeled with ABY™ fluorescent label, the third probe labeled with VIC™ fluorescent label, the fourth probe labeled with JUN™ fluorescent label, and/or the fifth probe labeled with Alexa Fluor™ AF647 fluorescent label. The first probe of the compositions can comprise the sequence of any one of SEQ ID NOs: 109-126, the second probe of the compositions can comprise the sequence of any one of SEQ ID NOs: 127-134, the third probe of the compositions can comprise the sequence of any one of SEQ ID NOs: 135-151, the fourth probe of the compositions can comprise the sequence of any one of SEQ ID NOs: 152-161, and/or the fifth probe of the compositions can comprise the sequence of SEQ ID NO: 162.

The compositions can comprise a positive control for amplification. The positive control can comprise a synthetic plasmid comprising an insert sequence of SEQ ID NO: 271.

Also provided are kits comprising sets of forward primers and reverse primers as disclosed herein. The kits can comprise a first probe, a second probe, a third probe, a fourth probe, and/or a fifth probe. The probes of the kits can comprise a label and a quencher.

Also provided are methods for detecting CT, MG, NG, and/or TV in a biological sample. The methods can comprise (a) combining a biological sample and any one of the compositions described herein to form a reaction mixture; (b) subjecting the reaction mixture to amplification conditions, thereby forming one or more amplification products; and (c) detecting at least one of the amplification products. The biological sample can be selected from a blood sample, a buccal sample, a urine sample, a semen sample, a vaginal secretion sample, and a vaginal swab. The biological sample can be from a human or from a non-human subject.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope.

The disclosure will be described and explained with additional specificity and detail through the use of the accompanying drawings in which:

FIGS. 1A, 1B, 1C, and 1D are amplification plots depicting the detection of different CT target sequences at different dilutions (1E5, 1E4, 1E3, 100, and 10 cp/rxn (copies per reaction)) of synthetic DNA. The detection of the RNase P target sequence amplification is also presented at single concentration of the target sequence. The data is represented as change in Rn (ΔRn) over cycle number (Cycle). FIGS. 1E, IF, 1G, and 1H are amplification plots depicting the detection of different NG target sequences at different dilutions (1E5, 1E4, 1E3, 100, and 10 cp/rxn (copies per reaction)) of synthetic DNA. The detection of the RNase P target sequence amplification is also presented at single concentration of the target sequence. The data is represented as change in Rn (ΔRn) over cycle number (Cycle). FIGS. 1I, 1J, 1K, and IL are amplification plots depicting the detection of different MG target sequences at different dilutions (1E5, 1E4, 1E3, 100, and 10 cp/rxn (copies per reaction)) of synthetic DNA. The detection of the RNase P target sequence amplification is also presented at single concentration of the target sequence. The data is represented as change in Rn (ΔRn) over cycle number (Cycle). FIGS. 1M, 1N, 1O, and 1P are amplification plots depicting the detection of different TV target sequences at different dilutions (1E5, 1E4, 1E3, 100, and 10 cp/rxn (copies per reaction)) of synthetic DNA. The detection of the RNase P target sequence amplification is also presented at single concentration of the target sequence. The data is represented as change in Rn (ΔRn) over cycle number (Cycle).

FIG. 2 depicts a table depicting the assay linearity and PCR efficiency for amplifying the target sequences of CT, NG, MG, and TV.

FIG. 3 depicts a table depicting the analytical sensitivity of the assays for detecting the target sequences of CT, NG, MG, and TV.

FIG. 4A depicts a table depicting the different conditions tested for the competitive interference study. FIG. 4B depicts a table depicting the hit rate for the competitive interference study. FIG. 4C depicts a table depicting a summary of the mean Cq and ΔCq values for the competitive interference study.

FIGS. 5A and 5B are a tables depicting positive calls From the CT(−)/NG(−) and unknown clinical specimens after testing the samples with the 5-plex assay. FIG. 5C is a table depicting the equivalent concordance for positive confirmed specimens with 10 k and 20 k ΔRn thresholds. A total of 37 individual clinical sample had been confirmed positively by third party confirmation methods and tested with STI panel—the 5-plex assay (10 samples for CT, MG and TV, and 7 for NG). *Initial Cq=33.3. **MG Sample #7 result is NG (+).

FIG. 6A depicts the nucleic acid sequence (SEQ ID NO: 271) of the insert for the amplification control used in the 5-plex assay. FIGS. 6B and 6C depict a concise map of the insert. FIG. 6D is a diagram depicting the map for the super plasmid CT_STI PC that is used to carry the insert of FIG. 6A.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

All publications and patent applications cited herein, as well as the Appendices attached hereto, are incorporated by reference in their entirety for all purposes to the same extent as if each were specifically and individually indicated to be so incorporated by reference. Although the present invention has been described in some detail by way of illustration and example for purposes of clarity and understanding, it will be apparent that certain changes and modifications may be practiced within the scope of the spirit and substance of this disclosure and of the appended claims.

Any headings and subheadings used herein are for organizational purposes only and are not meant to be used to limit the scope of the description or the claims.

### Overview of Compositions, Systems, and Kits for Detection of Target Sequences

Disclosed herein are compositions, kits, and methods for specifically detecting pathogen sequences, in particular STI pathogens Chlamydia trachomatis (CT), Neisseria gonorrhoeae (NG), Mycoplasma genitalium (MG), and Trichomonas vaginalis (TV).

The disclosed compositions, kits, and methods for detection of STI pathogens are for multiplex amplification of one or more target regions from the STI pathogens genomes. The multiplex amplification can comprise 4-plex amplification for amplifying one or more target regions from four different STI pathogens genomes. The multiplex amplification can comprise 5-plex amplification for amplifying one or more target regions from four different STI pathogens genomes and one or more target regions from a control gene.

When an example “embodiment” or a particular “assay” is described herein, it will be understood that the features of the embodiment may be applicable to a composition (e.g., the particular physical components of an assay such as primers and/or probes, or a primer-probe set), a kit (e.g., primers and/or probes and additional buffers, reagents, etc.), or a method (e.g., a process for detecting target nucleic acids) as appropriate. For simplicity, many embodiments are presented by describing “assays”, but it will be understood that the associated methods of using the assays are also intended to form part of this disclosure.

In “multiplex” amplification embodiments, the formation of a plurality of separate and different amplification products or amplicons can be tracked over time by measuring a signal in one or more detection channels. The signal can be emitted by a detectable label, optionally a fluorescent label, attached to a primer and/or probe that selectively hybridizes to the amplification product or amplicon. In some embodiments, each channel is calibrated to preferentially or selectively detect a corresponding amplification product or amplicon, and the signal in each channel is used as a measure of concentration of the corresponding amplification product or amplicon.

For example, in some embodiments of the multiplex assay, an amplification product or amplicon of the CT target region is detected in a first detection channel based on a first signal emitted by a first label attached to, or associated with, a first set of forward primers and reverse primers and/or first probe that selectively hybridizes to the amplification product or amplicon of the CT target region; an amplification product or amplicon of the MG target region is detected in a second detection channel based on a second signal emitted by a second label attached to, or associated with, a second set of forward primers and reverse primers and/or second probe that selectively hybridizes to the amplification product or amplicon of the MG target region; an amplification product or amplicon of the NG target region is detected in a third detection channel based on a third signal emitted by a third label attached to, or associated with, a third set of forward primers and reverse primers and/or third probe that selectively hybridizes to the amplification product or amplicon of the NG target region; and an amplification product or amplicon of the TV target region is detected in a fourth detection channel based on a fourth signal emitted by a fourth label attached to, or associated with, a fourth set of forward primers and reverse primers and/or fourth probe that selectively hybridizes to the amplification product or amplicon of the TV target region.

In some embodiments of the multiplex assay, the amplification product or amplicon of a positive control or reference sequence is detected in a fifth channel based on a fifth signal emitted by a fifth label attached to a fifth primer and/or fifth probe that selectively hybridizes to amplification product or amplicon of the positive control or reference sequence. An amplification product of ribonuclease P is detected in a fifth detection channel based on a fifth signal emitted by a fifth label attached to, or associated with, a fifth primer and/or fifth probe that selectively hybridizes to the ribonuclease P DNA amplification product.

In some embodiments, a background noise is detected in a sixth channel based on a signal from a negative control. In a further embodiment, the negative control is water, for instance.

In some embodiments, a multiplex assay according to some embodiments includes a 4-plex assay. In some embodiments, the multiplex assay is a 4-plex assay that includes a four-channel panels with each assay in a different channel. In some embodiments, the 4-plex assay includes assays for CT, MG, NG, and TV.

In some embodiments, a multiplex assay according to some embodiments includes a 5-plex assay. In some embodiments, the multiplex assay is a 5-plex assay that includes a five-channel panels with each assay in a different channel. In some embodiments, the 5-plex assay includes assays for CT, MG, NG, TV, and ribonuclease P (RNase P).

In some embodiments, the multiplex assay includes one forward primer, one reverse primer, and/or one probe for each of the CT, MG, NG, and TV. In some embodiments, the multiplex assay includes one forward primer, one reverse primer, and/or one probe for each of the CT, MG, NG, TV, and the RNase P assay. In some embodiments, the primers and probes have nucleotide sequences selected from the sequences shown in Tables 1 and 2 below.

The assays provide at least 99% strain coverage of the STI pathogens. As described in Table 6 below, the term “strain coverage” refers to a fraction or percentage (%) of known strains of the pathogen detected by the primer set and/or the primer and probe set of the assay.

Several STI pathogen panels are currently on the market for detecting CT, MG, NG, and/or TV with polymerase chain reaction (PCR)-based tests. The target sequences for these available assays do not offer at least about 99% strain coverage for each one of the CT, MG, NG, and/or TV STI pathogens.

As explained above, the conventional diagnostic assays may not detect the available strains of STI pathogens. This lack of resolution can prove problematic in attempts to track the spread and progression of the STI and/or requires more expensive and lengthy sequencing testing to identify the pathogen specifically.

### Example Assays and Associated Components

Embodiments disclosed herein include primers and optionally probes useful for the detection of four different species of STI pathogens in a sample (e.g., a biological sample). Such primers, oligonucleotides, and probes can be used in a nucleic acid assay (e.g., a singleplex or a multiplex assay) for detection and identification of one or more nucleic acid targets in a sample. The assays described herein demonstrate a high level of sensitivity, specificity, and accuracy. In some embodiments, an assay is designed to detect and differentiate between four different species of STI pathogens. For example, an assay may be configured to detect the presence of CT nucleic acid and nucleic acid of MG, NG, and/or TV within a biological sample.

In some embodiments, an assay includes differentially labelled probes where at least one probe is configured to associate with, or configured to hybridize to, amplicons of a CT sequence, at least one probe is configured to associate with, or configured to hybridize to, amplicons of a MG sequence, at least one probe is configured to associate with, or configured to hybridize to, amplicons of a NG sequence, and at least one probe is configured to associate with, or configured to hybridize to, amplicons of a TV sequence. In some embodiments, the assay also includes at least one probe is configured to associate with, or configured to hybridize to, amplicons of a control gene sequence. For example, an additional labelled probe configured to hybridize to RNase P may be further included in the assay.

In some embodiments, assays are configured to detect an amplification product of a particular target region by detecting a signal from a label (i.e., a detectable label) or other signal-generating process, where the signal indicates formation of the amplification product. In some embodiments, the label is attached to, or otherwise associated with, the corresponding forward primer and/or reverse primer used to generate the amplification product. Additionally, or alternatively, the label is attached to, linked, or otherwise associated with, either directly or indirectly, a probe that hybridizes to or associate with a probe-binding sequence within the target region. In some embodiments, the target region is the region shown in Table 7. In some embodiments, the label is an optically detectable label. Alternatively, the label may be detectable non-optically, such as, for example, electronically, electrically, or by using nuclear magnetic resonance (NMR) spectroscopy, sound, radioactivity, and the like.

In some exemplary processes for detecting CT, MG, NG, and TV, target regions in the pathogen genomes are first amplified by polymerase chain reaction (PCR). The reaction mixture includes a probe designed to target a sequence of CT, a probe designed to target a sequence of MG, a probe designed to target a sequence of NG, a probe designed to target a sequence of TV, and a probe designed to target a sequence of RNase P. Each probe type is also associated with a different dye channel to enable differential detection. In an example embodiment, the CT probe includes a FAM™ dye label, the MG probe includes a ABY™ dye label, the NG probe includes a VIC™ dye label, the TV probe includes a JUN™ dye label, and the RNase P probe includes a AF647 dye label. The probes may be configured as TaqMan probes, which are known in the art and described in greater detail below. When the probe is able to hybridize to a target downstream from a primer, the exonuclease activity of the polymerase during subsequent primer extension separates the dye label from the quencher to increase the dye signal.

Disclosed herein are primers and probes that hybridize to target regions of STI pathogen genomes, such as to the target regions of the CT, MG, NG, and TV genomes, as well as primers and probes that hybridize to a control gene, such as to RNase P. The primers and probes may be utilized in an assay that can detect the four pathogens CT, MG, NG, and TV. The disclosed primers and probes can detect the STI pathogens at at least about 95% strain coverage. In some embodiments, the disclosed primers and probes can detect the STI pathogens at least about 95% strain coverage, at least about 96% strain coverage, at least about 97% strain coverage, at least about 98% strain coverage, at least about 99% strain coverage, at least about 99.5%, or at 100% strain coverage.

In some embodiment, the primers and probes that hybridize to the target regions of STI pathogen genomes include one or more sets of those shown in Table 2. Table 2 lists exemplary CT, MG, NG, and TV forward primers and CT, MG, NG, and TV reverse primers (corresponding to SEQ ID NO: 1-SEQ ID NO: 108), and CT, MG, NG, and TV probes (corresponding to SEQ ID NO:109-SEQ ID NO:162) that may be utilized in conjunction with the corresponding CT, MG, NG, and TV forward and reverse primers. For example, in some embodiments, an assay can include a set of forward primers and reverse primers. In particular, the “set” of forward primers and reverse primers can correspond to one or more rows of primers in Table 2 that listed for the same STI pathogen.

In some embodiments, the multiplex assay further includes a fifth set of primers and probes. In some embodiments, the fifth set of primers and probes include RNase P primers and probes. In some embodiments, the RNase P primers and probes include the sequences shown in Table 2.

In some embodiments, multiple assays each corresponding to a different organism can be combined to create an assay panel designed to specifically detect the STI pathogens.

In embodiments, each probe includes a detectable label (e.g., a fluorescent reporter molecule) and a quencher molecule capable of quenching the fluorescence of the reporter molecule. The probe can comprise a label selected from FAM™, VIC™, ABY™, JUN™; FITC; 6-carboxy-4′,5′-dichloro-2′,7′-dimethoxy fluorescein (JOE™); 6-carboxy-1,4-dichloro-2′,7′-dichloro-fluorescein (TET™); 6-carboxy-1,4-dichloro-2′,4′,5′,7′-tetra-chlorofluorescein (HEX™); Alexa Fluor™ fluorophores (e.g., AF647), NED™, and BODIPY™ fluorophores. Typically, the detectable label and quencher molecule are part of a single probe. The probe can comprise a minor groove-binder (MGB).

In some embodiments, the multiplex assay further includes a positive control for amplification. In an embodiment, the positive control for amplification includes templates specific to target regions of the CT, MG, NG, and TV genomes, and human RNase P regions. In some embodiments, the positive control includes the sequences shown in Table 9. In some embodiments, the positive control for amplification comprises a plasmid with target regions of the CT, MG, NG, and TV genomes, and human RNase P regions.

### Sample Collection

The disclosed compositions, kits, and methods are configured to detect STI pathogen nucleic acid from a sample. According to the instant disclosure, the disclosed compositions, kits, and methods are configured specifically for detection of STI pathogens in a biological sample. Accordingly, the biological sample can comprise a blood sample, a buccal sample, a urine sample, a semen sample, a vaginal secretion sample, and a vaginal swab. In some embodiments, the sample is a human sample. In some embodiments, if and where relevant to the particular STI pathogen, the sample is a non-human sample. For instance, the sample may be from a non-human species such as a rodent (e.g., prairie dogs, squirrels, chinchillas), a carnivore (e.g., dogs, cats), an insectivore (e.g., hedgehogs, shrews), a non-human primate (e.g., monkeys, apes). In most instances, the STI pathogen is detected by analysis of a blood sample, a buccal sample, a urine sample, a semen sample, a vaginal secretion sample, or a vaginal swab.

The sample can be collected by a healthcare professional in a healthcare setting, but in some instances, the sample is collected by the patient themselves or by an individual assisting the patient in self-collection. Swabs are often used by a healthcare professional in a healthcare setting. Other samples can similarly be obtained in a healthcare setting with the assistance or oversight of a healthcare professional. However, in some instances, self-collection of a sample can be more efficient and can be done outside of a healthcare setting.

The swab storage tube/device or extracted sample collection tube/device may be a component of a self-collection kit having instructions for use, such as sample collection instructions, sample preparation or storage instructions, and/or shipping instructions.

In other embodiments, the sample is collected—whether by self-collection or assisted/supervised collection—in a sterile tube or specifically-designed collection device. The collection tube/device may be a component of a self-collection kit having instructions for use, such as sample collection instructions, sample preparation or storage instructions, and/or shipping instructions. The sample can be collected directly into a scalable container without any preservation solution or other fluid or substance in the container prior to receipt of the sample within the container or because of closing/scaling the container.

In some embodiments, the sample is pre-treated prior to use. This can include, for example, heating the sample, such as by placing the raw sample on a heat block/water bath set to a temperature of 95° C. for 30 minutes, followed by combining the heat-treated sample with a buffer or lysis solution. The buffer or lysis solution can include, for example, any nucleic-acid-amenable buffer such as tris-borate-ethylenediaminetetraacetic acid (TBE) and may further include a detergent and/or emulsifier such as the polysorbate-type nonionic surfactant Tween®-20.

In some embodiments, a nucleic acid fraction of the sample (e.g., obtained by a swab or a draw) can be extracted and used for downstream analysis, such as qPCR. In some embodiments, the sample is a vaginal swab or semen. As provided above, the sample can be self-collected (e.g., within a sterile swab collection device) or collected from the patient by any other individual in proximity to the patient. In some embodiments, the swab or draw is collected directly into a scalable container without any preservation solution or other fluid or substance in the container prior to receipt of the sample or because of closing/sealing the container. The disclosed embodiments for detecting nucleic acid from a sample can be adapted to detect nucleic acid directly from the sample, or in alternative embodiments, the sample can undergo a specific DNA purification and/or extraction step prior to its use in a detection assay (e.g., qPCR). Thus, it should be appreciated that in some embodiments, a patient sample (e.g., a swab) can directly serve as sample input for subsequent downstream analyses, such as PCR, and this can be accomplished, in some embodiments, with no nucleic acid purification and/or extraction step prior to its use. In some embodiments, the sample used in subsequent downstream analyses is a heat-treated sample as described herein.

In some implementations, the nucleic acid may be detected directly from a raw or crude sample. For example, a raw sample can be collected from the patient and heat-treated, such as by placing the raw sample on a heat block/water bath set to a temperature of about 95° C. for 30 minutes. The heating step can provide many benefits, including, for example, denaturing nucleases such as RNase within the raw sample that may interfere with accurate assessments of DNA presence. Heating a raw sample can also make the sample more amenable to manipulation with laboratory equipment such as pipettes. The high heat can also cause thermal disruption of any prokaryotic and eukaryotic cells present in the sample and can also increase accessibility to any nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of nucleic acid within the sample via nucleic acid amplification reactions (e.g., PCR, qPCR, or the like). The lysis solution can include a nucleic-acid-amenable buffer such as TBE (and/or suitable alternative known in the art) combined with a detergent and/or emulsifier such as Tween®-20, the polysorbate-type nonionic surfactant (and/or suitable alternative known in the art). The detergent and/or emulsifier can promote better mixing of the reagents and may also act to increase accessibility to any nucleic acid within the sample.

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the sample/buffer mixture. In other embodiments, the sample is directly combined with a buffer/detergent mixture.

As a nonlimiting example, a set of patient samples can be prepared as compositions for downstream analysis and detection of a target sequence by adding a volume of heat-treated sample for each patient into one (or a plurality) of wells in a multi-well plate. A volume of a buffer/detergent mixture (e.g., TBE+Tween®-20) can then be added to each well containing a patient sample. Alternatively, a multi-well plate can be loaded with a volume of a buffer/detergent mixture into which a volume of heat-treated sample is added. Once combined, this probative template solution can be used immediately or stored for later analysis. Such probative template solutions can also be combined with PCR reagents (e.g., buffers, deoxynucleotide triphosphates (dNTPs), master mixes, etc.) prior to or after storage.

In some embodiments, a sample is obtained from multiple organisms (e.g., a plurality of individuals or patients) and the multiples samples are pooled together to make a single pooled sample for testing. In some embodiments, a sample may be obtained from at least two different organisms or individuals for pooling together to form a single sample for testing. In some embodiments, a sample may be obtained from between 2 to 10 different organisms or individuals for pooling together to form a single sample for testing. In some embodiments, a sample may be obtained from 2, 3, 4, 5, 6, 7, 8, 9, or 10 different organisms or individuals for pooling together to form a single sample for testing. In some embodiments, a sample may be obtained from up to and including six different organisms or individuals for pooling together to form a single sample for testing. For example, a sample used for testing, according to the methods and compositions described herein, may comprise a multiplicity of samples obtained from different organisms or individuals (e.g., 2, 3, 4, 5, or 6 different individuals) which are combined together to form a single “pooled” sample used for subsequent detection of the STI pathogens.

### Nucleic Acid Amplification and Detection

Amplified or amplification products (“amplicons”) resulting from use of one or more embodiments described herein can be generated, detected, and/or analyzed using any suitable method and on any suitable platform. In some embodiments, the STI pathogen or other target organism is detected by analysis of samples obtained from one or more subjects.

In some embodiments, the nucleic acid assays as described herein can be used to detect, identify, characterize, quantify, or otherwise measure one or more nucleic acid targets in a sample. In some embodiments, the nucleic acid targets may be single-stranded, double-stranded, or any other nucleic acid molecule of any size or conformation. Optionally, the nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods, including methods discussed below for detecting nucleic acid targets in a sample. In some embodiments, the PCR assays can be real time PCR or quantitative (qPCR) assays. In some other embodiments, the PCR assays can be end point PCR assays. Nucleic acid markers may be detected by any suitable means, including means that include nucleic acid amplification (e.g., thermal cycling amplification methods including PCR, and other nucleic acid amplification methods; isothermal amplification methods, including LAMP, etc.) and any other method that can be used to detect the presence of nucleic acid markers indicative of a disease-causing organism in a sample.

In some embodiments, the primers described herein are used in nucleic acid assays at a concentration in the range of about 100 nM to 1 mM (e.g., 300 nM, 400 nM, 500 nM, etc.), including all concentration amounts and ranges in between. In some embodiments, the probes described herein are used in a nucleic acid assay at a concentration in the range of about 50 nM to 500 nM (e.g., 75 nM, 125 nM, 250 nM, etc.), including all concentration amounts and ranges in between.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels may include, but are not limited to, 6FAM, ABY™, VIC™, JUN™, FAM™. Suitable quenchers may include, but are not limited to, MGB, QSY® (e.g., QSY7 and QSY21), BHQ™ (Black Hole Quencher), and DFQ (Dark Fluorescent Quencher).

In some embodiments, control sequence primers and/or probes (e.g., labeled probes), such as for amplification and/or detection of the STI pathogen the control target sequences, are included in the multiplex assays using primer/probe sequences disclosed herein (and may be included as singleplex assays as well).

In some embodiments, control sequence primers and/or probes (e.g., AF647-labeled probes), such as for amplification and/or detection of human RNase P control sequences, are included in the multiplex assays using primer/probe sequences disclosed herein (and may be included as singleplex assays as well).

In some embodiments, array-formatted assays can be run as a singleplex assay or as multiplex assays. In some embodiments, a panel of different assays may be formatted onto an array or a multi-well plate. In some embodiments, the panel can include one or more assays containing at least one primer-set and a probe of Tables 2 and 3 present in at least one well of an array or a well of a multi-well plate. In some embodiments, the panel includes assays for CT, MG, NG, and TV, and RNase P. In some embodiments, the disclosed methods include using the panel to profile microorganisms present in a sample taken from an organism (e.g., human) and determining the profile of microbiota present in the organism's sample. Optionally, the disclosed methods can include diagnosing an infection present in an organism (e.g., human) from which a sample is taken.

In some embodiments, the panel of qPCR assays can be used simultaneously to test a single patient sample or a single pooled sample comprising multiple patient samples, with each assay run in parallel in array format (“array formatted”). Optionally, different qPCR assays specific for each of the following target assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use.

In some embodiments, the panel of qPCR assays includes at least one qPCR assay for detecting CT, MG, NG, or TV. In some other embodiments, the panel of qPCR assays includes at least one qPCR assay for detecting one or more of CT, MG, NG, and TV. In some other embodiments, the panel of qPCR assays includes at least one qPCR assay for detecting one or more of CT, MG, NG, TV, and RNasesP. In some other embodiments, the panel of qPCR assays includes at least one qPCR assay for detecting CT, MG, NG, TV, and RNasesP. Each qPCR assay can include a forward primer, a reverse primer, and a probe for each target.

In some other embodiments, the primers and/or probes provided herein can be used to amplify one or more specific target sequences present in CT, MG, NG, or TV.

The primer and probe sequences described herein need not have 100% homology/identity to their targets to be effective, though in some embodiments, homology is substantially 100% or exactly 100%. In some embodiments, one or more of the disclosed primer and/or probe sequences have a homology to their respective target of at least about 50%, at least about 60%, at least about 70%, at least about 75%, at least about 80%, at least about 85%, at least about 90%, at least about 95%, at least about 97%, at least about 98%, at least about 99%, at least about 99.9%, or up to substantially 100% or exactly 100%. Some combinations of primers and/or probes may include primers and/or probes each with different homologies to their respective targets, and the homologies may be, for example, within a range with endpoints defined by any two of the foregoing values.

PCR and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as those listed in Table 1, may require less or less extensive thermal cycling than does PCR, or require no thermal cycling. Such isothermal amplification methods are also contemplated for use with the assay compositions, kits, and methods described herein.

TABLE 1
Alternative DNA Amplification Processes

| | |
|---|---|
| LAMP | Loop-mediated isothermal amplification (LAMP) is a single tube |
| | technique for the amplification of DNA. It typically uses 4-6 primers, which |
| | form loop structures to facilitate subsequent rounds of amplification. |
| HDA | Helicase-dependent amplification (HDA) uses the double-stranded |
| | DNA unwinding activity of a helicase to separate strands for in vitro DNA |
| | amplification at constant temperature. |
| RCA | Rolling circle amplification (RCA) starts from a circular DNA |
| | template and a short DNA primer to form a long single-stranded molecule. |
| MDA | Multiple displacement amplification (MDA) is a technique that |
| | initiates when multiple random primers anneal to the DNA template and the |
| | polymerase amplifies DNA at a constant temperature. |
| WGA | When MDA is used to amplify DNA from a whole genome of a cell it |
| | is called whole genome amplification (WGA). |
| RPA | Recombinase polymerase amplification (RPA) is a low-temperature |
| | DNA amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

In some embodiments, different assay products (e.g., amplicons from CT, MG, NG, TV, and RNasesP) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety. In some embodiments, specific combinations of labels are used to differentiate between CT, MG, NG, TV, and the positive controls. For example, CT, MG, NG, TV, and the positive control RNaseP may be detected and differentiated from each other using the different labels such that the label is detectable only in the presence—and amplification—of the associated sequence.

In some embodiments, the amplifying step can include performing qPCR, as that term is defined herein. qPCR is a sensitive and specific method for detecting and optionally quantifying amounts of starting nucleic acid template in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Suitable labels are described herein.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, one or more probe sequences are also included in the singleplex reaction volume.

In some array-formatted multiplex embodiments, the reaction volume within the selected tubes, grooves, channels, wells, cavities, sites, or features contains multiple (e.g., 2, 3, 4, 5, 6, etc.) forward and reverse primer sequences and/or multiple probe sequences. For instance, exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety).

In some embodiments (e.g., in the well-known and widely used TaqMan™ line of qPCR assays), detecting an amplification product includes detecting a signal emitted by a fluorescent label attached to the 5′ end of a cleavable probe that selectively hybridizes to the amplification product during amplification. The cleavable probe further includes a quencher that quenches the fluorescent label to a ‘baseline’ fluorescence level. The 5′ end of the cleavable probe is cleaved by the polymerase during the extension step, resulting in the separation of the fluorescent label from the quencher and a corresponding increase in fluorescence over baseline. TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. Thus, the oligonucleotide probe includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule and a continuing increase in fluorescence over baseline is measured at each cycle. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

In some embodiments, a passive reference dye, such as ROX™ is included in the reaction mixture. The metric “Rₙ” is optionally used to track progress of the amplification reaction and to determine the amount of target sequence originally present in the reaction mixture prior to amplification. Rₙ can be calculated as the fluorescence of the reporter dye divided by the fluorescence of a passive reference dye present in the reaction mixture; i.e., Rₙ is the reporter signal normalized to the fluorescence signal of the passive reference dye. In some embodiments, Rₙ is plotted against PCR cycle number. In some embodiments, ARₙ (calculated as Rₙ minus the baseline) can be plotted against PCR cycle number. In some embodiments, an amplification plot shows the variation of log (ARₙ) with PCR cycle number. Cₜ (threshold cycle) is the intersection between an amplification curve and a threshold line. The lower the Cₜ value for a given amplification product, the earlier the amplification is detectable and the higher the absolute amount, and the relative concentration, of the corresponding target sequence originally present in the reaction mixture. In some embodiments, cutoffs for Cₜ are used to determine whether a target sequence was originally present or absent in the reaction mixture prior to amplification. For example, in some embodiments a target sequence is determined to be present if the Cₜ value is less than or equal to 37.

In various embodiments, a singleplex or multiplex qPCR can include a single TaqMan assay associated with a locus-specific sequence, or multiple TaqMan assays respectively associated with a plurality target sequences in a multiplex format, or multiple TaqMan assays respectively associated with a plurality of loci in a multiplex format. As a non-limiting example, a triplex reaction can include FAM™ (emission peak at 517 nm), VIC™ (emission peak at 551 nm), and JUN™ (emission peak at 617 nm) dyes. As a non-limiting example, a 4-plex reaction can include FAM™ (emission peak at 517 nm), VIC™ (emission peak at 551 nm), ABY™ (emission peak at 580 nm), and JUN™ (emission peak at 617 nm) dyes. As a non-limiting example, a 5-plex reaction can include FAM™ (emission peak at 517 nm), VIC™ (emission peak at 551 nm), ABY™ (emission peak at 580 nm), JUN™ (emission peak at 617 nm) and AF647 (emission peak at 665 nm) dyes. In some embodiments, each dye is associated with one or more target sequences. In some embodiments, one or more dyes are quenched by MGB or a QSY® quencher (e.g., QSY7 or QSY21). In some embodiments, each multiplex reaction allows 12 or 13 targets to be amplified and tracked real-time within a single reaction vessel. In some embodiments, up to 2, 4, 6, 8, 10, 12, or 14 targets are amplified and tracked real-time within a single reaction vessel, using any combination of detectable labels disclosed herein or otherwise known to those of skill in the art. The reporter dyes are optimized to work together with minimal spectral overlap for improved performance. Any combination of dyes described herein can additionally be combined with other dyes (e.g., Mustang Purple™ (emission peak 654 nm) or one or more Alexa Fluors® (e.g., AF647 and AF676)), for use in monitoring fluorescence of a control or for use in a non-emission-spectrum-overlapping 5-plex assay. In addition, the QSY® quencher is fully compatible with probes that have minor-groove binder (MGB) quenchers.

Where multiple detection channels are utilized, it is desirable to minimize crosstalk between fluorescence reporters and select reporters that avoid excessive spectral overlap. One example of an assay that includes 5 detection channels incorporates the dyes FAM™, ABY™, VIC™, and JUN™, along with Mustang Purple™ (emission peak of 654 nm) or an appropriate Alexa Fluor®, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs. Additional examples of multiplex assays (including related dye compounds, compositions, methods, and kits) are described in International Publication No. WO 2022/020731 A2, published Jan. 27, 2022 and titled “Compositions, Systems and Methods for Biological Analysis Involving Energy Transfer Dye Conjugates and Analytes Comprising the Same”, which is incorporated herein by this reference in its entirety.

Detector probes may be associated with alternative quenchers, including without limitation, dark fluorescent quencher (DFQ), black hole quenchers (BHQ™), Iowa Black, QSY® quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM™); 5-Hydroxy Tryptamine (5-HAT™); 6-JOE; 6-carboxyfluorescein (6-FAM™); Mustang Purple™, VIC™, ABY™, JUN™; FITC™; 6-carboxy-4′,5′-dichloro-2′,7′-dimethoxy, fluorescein (JOE™)); 6-carboxy-1,4-dichloro-2′,7′-di chloro-fluorescein (TET™); 6-carboxy-1,4-dichloro-2′,4′,5′,7′-tetra-chlorofluorescein (HEX™); Alexa Fluor™ fluorophores (e.g., 350, 405, 430, 488, 500, 514, 532, 546, 555, 568, 594, 610, 633, 635, 647, 660, 680, 700, 750); BODIPY™ fluorophores (e.g., 492/515, 493/503, 500/510, 505/515, 530/550, 542/563, 558/568, 564/570, 576/589, 581/591, 630/650-X, 650/665-X, 665/676, FL, FL ATP, Fl-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, 1AEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethylrhodamine), Tetramethylrhodamine (TRITC), WT), Texas Red, Texas Red-X, among others as would be known to those of skill in the art.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30(9): e37), Hayashi et al. (Nucleic Acids Res. 1989 May 11; 17(9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

In some embodiments, the amplicon is labeled by incorporation of or hybridization to labeled primer. In some embodiments, the amplicon is labeled by hybridization to a labeled probe. In some embodiments, the amplicon is labeled by binding of a DNA-binding dye. In some embodiments, the dye may be a single-strand DNA binding dye. In other embodiments, the dye may be a double-stranded DNA binding dye. In other embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In one exemplary embodiment, the labeled amplicon is detected using capillary electrophoresis. In another embodiment, the labeled amplicon is detected using qPCR. In some embodiments, a plurality of different amplicons is formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In some embodiments, the plurality of amplicons can be differentially labeled. In some embodiments, each of the plurality of amplicons produced during amplification is labeled with a different label.

Optionally, in some embodiments, a RNase P assay is included in the kit. Exemplary primers and probes for the RNase P assay can include sequences of a forward primer including SEQ ID NO. 107, a reverse primer including SEQ ID NO. 108, and a probe including SEQ ID NO. 162, although those having skill in the art should appreciate that other RNase-P-specific primers and/or probes could be used.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), the QuantStudio 5 DX RealTime PCR System (QS5DX), or the QuantStudio 7 Flex RealTime PCR System (QS7Flex), from Thermo Fisher Scientific.

In some embodiments, the systems, compositions, methods, and devices used for nucleic acid amplification comprise a “point-of-service” (POS) system. In some embodiments, samples may be collected and/or analyzed at a “point-of-care” (POC) location. In some embodiments, analysis at a POC location typically does not require specialized equipment and has rapid and easy-to-read visual results. In some embodiments, analysis can be performed in the field, in a home setting, and/or by a lay person not having specialized skills. In certain embodiments, for example, the analysis of a small-volume clinical sample may be completed using a POS system in a short period of time (e.g., within hours or minutes).

Optionally, a POS system is utilized at a location that is capable of providing a service (e.g., testing, monitoring, treatment, diagnosis, guidance, sample collection, verification of identity (ID verification), and other services) at or near the site or location of the subject. A service may be a medical service or it may be a non-medical service. In some situations, a POS system provides a service at a predetermined location, such as a subject's home, school, or work, or at a grocery store, a drug store, a community center, a clinic, a doctor's office, a hospital, an outdoor triage tent, a makeshift hospital, a border check point, etc. A POS system can include one or more point of service devices, such as a portable STI pathogen detector. In some embodiments, a POS system is a point of care system. In some embodiments, the POS system is suitable for use by non-specialized workers or personnel, such as nurses, police officers, civilian volunteers, or the patient.

In certain embodiments, a POC system is utilized at a location at which medical-related care (e.g., treatment, testing, monitoring, diagnosis, counseling, etc.) is provided. A POC may be, e.g., at a subject's home, work, or school, or at a grocery store, a community center, a drug store, a doctor's office, a clinic, a hospital, an outdoor triage tent, a makeshift hospital, a border check point, etc. A POC system is a system which may aid in, or may be used in, providing such medical-related care, and may be located at or near the site or location of the subject or the subject's health care provider (e.g., subject's home, work, or school, or at a grocery store, a community center, a drug store, a doctor's office, a clinic, a hospital, etc.).

In embodiments, a POS system is configured to accept a clinical sample obtained from a subject at the associated POS location. In embodiments, a POS system is further configured to analyze the clinical sample at the POS location. In embodiments, the clinical sample is a small volume clinical sample. In embodiments, the clinical sample is analyzed in a short period of time. In embodiments, the short period of time is determined with respect to the time at which sample analysis began. In embodiments, the short period of time is determined with respect to the time at which the sample was inserted into a device for the analysis of the sample. In embodiments, the short period of time is determined with respect to the time at which the sample was obtained from the subject.

In some embodiments, a POS system or a POC system can include the amplification-based methods, compositions and kits disclosed herein, including any of the described assays and/or assay panels. Such assays are contemplated for use with both thermal cycling amplification workflows and protocols, such as in PCR, as well as isothermal amplification workflows and protocols, such as in LAMP.

In some embodiments, a POS or a POC system comprises self-collection of a biological sample. In some embodiments, the self-collection may comprise the use of a self-collection kit and/or device, such as a swab or a tube. In some embodiments, the self-collection kit comprises instructions for use, including collection instructions, sample preparation or storage instructions, and/or shipping instructions. For example, the self-collection kit and/or device may be used by an individual, such as lay person, not having specialized skills or medical expertise. In some embodiments, self-collection may be performed by the patient themselves or by any other individual in proximity to the patient, such as but not limited to a parent, a care giver, a teacher, a friend, or other family member.

Notably, in some embodiments, the nucleic acid amplification protocol can be configured for rapid processing (e.g., in less than about 45 minutes) and high throughput, allowing for a minimally invasive method to quickly screen large numbers of individuals in a scalable way. This can be particularly useful to perform asymptomatic testing (e.g., before termination of pregnancy, IUD insertion, or any other procedure breaking the cervical barrier, for patients with new or multiple sexual partners, for adult women (where CT can occur without symptoms), and if the patient is HIV positive and/or has a sexual partner who is HIV positive, etc.) or for epidemiological purposes. The disclosed embodiments can also beneficially provide a lower cost sample collection system and method that enables self-collection (reducing health care professional staffing needs) using a low-cost collection device. This eliminates the requirements for swabs, buffers, transmission media (or other specialized transport medium), and the like. The disclosed embodiments also allow for a reduction in Personal Protective Equipment (PPE) requirements and costs. Because the reagents and methods are streamlined (e.g., no precursor nucleic acid purification and/or extraction step), there is a reduced use of nucleic acid preparation plastics which brings a coincident reduction in reagent costs and inventory costs. There is also a beneficial reduced dependence on supply-constrained items, and the compatibility of these methods and kit components with existing equipment improves the flexibility and simplicity of their implementation to the masses. Overall, such embodiments allow for a less expensive assay that can be accomplished more quickly from sample collection through result generation.

Some embodiments relate to kits containing one or more of the primers and/or probes disclosed in Tables 2 and 3. In exemplary embodiments, a 5-plex assay kit includes a STI pathogen multiplex assay panel including a solution of primers at about 300 nM, probes at about 250 nM, RNase P primers at about 300 nM, and RNase P probes at about 150 nM. In exemplary embodiments, the kit further includes a positive control for amplification, or quantitative control.

Optionally, the kit can further include a master mix. In some embodiments, the master mix is a PCR master mix, for instance TaqPath™ Infectious Disease 1-Step Multiplex Master Mix (No ROX™) (Thermo Fisher Scientific, Waltham, MA). In some embodiments, the kit includes primers, probes, and master mix sufficient to constitute a reaction mixture supporting amplification of at least one or more target regions from the STI pathogens.

In exemplary embodiments, sample preparation is accomplished with the KingFisher™ Flex Purification System (Thermo Fisher Scientific, Catalog No. 5400610) and a MagMAX™ Viral/Pathogen II Nucleic Acid Isolation Kit (Thermo Fisher Scientific, Catalog No. A48383).

In some array-based embodiments, two or more different qPCR assays (each containing a forward primer, a reverse primer, and optionally a probe) are used in a single well, cavity, site or feature of the array and products of each assay can be independently detected. For example, different assay products may be discriminated optically (e.g., using different labels present in components each assay) or using some other suitable method, including as described in U.S. Patent Publication No. 2019/0002963, incorporated by reference herein. In some embodiments, at least one primer of each assay contains an optically detectable label that can be discriminated from the optical label of at least one other assay.

In some embodiments, optimal amplification and detectability for the STI pathogen genomes is achieved by adding a master mix to the reaction volume prior to amplification. The master mix optionally includes a polymerase, nucleotides, buffers, and salts. In some embodiments (particularly multiplex assays), the reaction volume includes TrueMark™ Infectious Disease 1-Step Multiplex Master Mix (No ROX).

Exemplary sequences for forward and reverse primers and probes are presented in Tables 2 and 3. The exemplary dye assignments and primer and probe concentration for the 5-plex multiplex assay are presented in Tables 4 and 5. The strain coverage for the select genes of the STI pathogens are presented in Table 6. Exemplary target sequences for the STI pathogens are presented in Table 7. Exemplary amplicons are presented in Table 8. Table 9 lists the sequence of the amplification control insert.

TABLE 2
Primers and probes and their SEQ ID NOs detecting the STI pathogens in the 5-plex
multiplex assay.

| | | SEQ ID | | SEQ |
|---|---|---|---|---|
| Organism name | Forward | NO: | Reverse | ID NO: |
| Chlamydia | TCTGGAGATGATTCAGG | 1 | CGCACGGCAGAAAGGA | 2 |
| trachomatis | AAGTGTCT | | TATCTT | |
| Chlamydia | CTCAATCTACAGACGCC | 3 | CTGTTTCGGATATATCG | 4 |
| trachomatis | TCTACAG | | CCATTGACT | |
| Chlamydia | CTCAATCTACAGACGCC | 5 | CTGTTTCGGATATATCG | 6 |
| trachomatis | TCTACAG | | CCATTGACT | |
| Chlamydia | TCTGGAGATGATTCAGG | 7 | CGCACGGCAGAAAGGA | 8 |
| trachomatis | AAGTGTCT | | TATCTT | |
| Chlamydia | TGGCGATATATCCGAAA | 9 | CGCACGGCAGAAAGGA | 10 |
| trachomatis | CAGAATCC | | TATCTT | |
| Chlamydia | GCAGCAGCACTCAATTC | 11 | GGTCCAGGGCCGAATA | 12 |
| trachomatis (all | TCTAAGA | | CAC | |
| serovars) | | | | |
| Chlamydia | CTCAAGCCGGGCCTAGT | 13 | AGAGGATGGCGTTGGT | 14 |
| trachomatis (all | AG | | GATG | |
| serovars) | | | | |
| Chlamydia | CGCCTCTACAGATACAA | 15 | CCAGCTCCGTCAGAGTC | 16 |
| trachomatis (all | GCGATAT | | TTTG | |
| serovars) | | | | |
| Chlamydia | CAGAACATGCTCTTCAT | 17 | GCTAAGTCAGGATGGC | 18 |
| trachomatis (all | CCTAAAGC | | CCATTT | |
| serovars) | | | | |
| Chlamydia | CAGAACATGCTCTTCAT | 19 | GCTAAGTCAGGATGGC | 20 |
| trachomatis (all | CCTAAAGC | | CCATT | |
| serovars) | | | | |
| Chlamydia | GCTTTGCAAGAGCCCTA | 21 | CTAGGATGATTAGGGTG | 22 |
| trachomatis (all | TAATGAAC | | TTGTTTACGT | |
| serovars) | | | | |
| Chlamydia | ACTATATTCATCACATGT | 23 | GCAGAAGAGGCTTTTCG | 24 |
| trachomatis (all | GCGAAGCT | | CTTTT | |
| serovars) | | | | |
| Chlamydia | CAAGACCTTTTTTCGCTG | 25 | CCCCTTCTAAAAGAGCT | 26 |
| trachomatis (all | TTTTTGTC | | CGAATGAT | |
| serovars) | | | | |
| Chlamydia | GATGTCGCTTCTCGCTAT | 27 | TGACTCCGTATTTCTCT | 28 |
| trachomatis (all | CATATCA | | GCTATCTCT | |
| serovars) | | | | |
| Chlamydia | GGTGGGATTAGTGGTGA | 29 | AGCAGGAATAGCTAGA | 30 |
| trachomatis sv L | TAGGACTA | | TAGATGGGATTT | |
| (LGV specific) | | | | |
| Chlamydia | GGTGGGATTAGTGGTGA | 31 | AGCAGGAATAGCTAGA | 32 |
| trachomatis sv L | TAGGACTA | | TAGATGGGATTT | |
| (LGV specific) | | | | |
| Chlamydia | GGTGGTGCGCTTTATGC | 33 | CCGCAGTCTCCAGACAT | 34 |
| trachomatis sv L | AAA | | ATTGTTAA | |
| (LGV specific) | | | | |
| Chlamydia | GATAGCAATACCGCTGC | 35 | CCCAGGCAGCAGAGTT | 36 |
| trachomatis sv L | TCATG | | ATTTAGAAA | |
| (LGV specific) | | | | |
| Mycoplasma | TTTTACTCAGTTTTATCC | 37 | AGTCATACCAAAGACT | 38 |
| genitalium | CAGGCTTCTT | | AAAAAGCGTTATCA | |
| Mycoplasma | TTTTACTCAGTTTTATCC | 39 | AGTCATACCAAAGACT | 40 |
| genitalium | CAGGCTTCTT | | AAAAAGCGTTATCA | |
| Mycoplasma | CCAGGTGTGAATTGTAT | 41 | GTCTTGTCTACAGAGGG | 42 |
| genitalium | GGTTTGAAA | | CAATGATT | |
| Mycoplasma | CCGTCCCCTAATGAAAT | 43 | CCTCACTTCATTGCGCT | 44 |
| genitalium | ACTGAAAAGA | | TATAAATAGGT | |
| Mycoplasma | GCTTTAAAACCGCTTGTC | 45 | GGCAATTGGGCTAAAC | 46 |
| genitalium | AGTTCT | | AAAAGGTTA | |
| Mycoplasma | ATTGTTATCAGCCCCTTA | 47 | CTTGACCAAAAACACC | 48 |
| genitalium | AGCTTGT | | AATAAAGGGTAA | |
| Mycoplasma | GAAGATTGCTTGTGTTGT | 49 | AACAGTGTGAACAACC | 50 |
| genitalium | TCATGTCA | | AGTGGAATA | |
| Mycoplasma | GGTGGATCTGATTCAAT | 51 | CACGTAATGAGGTTGAT | 52 |
| genitalium | GCTAATGC | | CTGGTGTT | |
| Neisseria | CCATACAGTCCGATATG | 53 | GCCTTTGGGTTTACGTC | 54 |
| gonorrhoeae | CCAATAGA | | CATAAGAA | |
| Neisseria | GCATTGCCGCAACCAAA | 55 | CGATATAACTTCGTAGC | 56 |
| gonorrhoeae | TTAAAAG | | GTTCTTGCT | |
| Neisseria | CATTGCCGCAACCAAAT | 57 | GCTATCACGAGCGATAT | 58 |
| gonorrhoeae | TAAAAGAA | | AACTTCGT | |
| Neisseria | GTAGCAAATAGTGATGT | 59 | GCCTTTGGGTTTACGTC | 60 |
| gonorrhoeae | CGCCGTAT | | CATAAGAA | |
| Neisseria | GATGTCGCCGTATTAAC | 61 | GCCTTTGGGTTTACGTC | 62 |
| gonorrhoeae | ACCCATA | | CATAAGAA | |
| Neisseria | GCATTGCCGCAACCAAA | 63 | CGATATAACTTCGTAGC | 64 |
| gonorrhoeae | TTAAAAG | | GTTCTTGCT | |
| Neisseria | CGGCAAAAAGACACGGT | 65 | GCTATCACGAGCGATAT | 66 |
| gonorrhoeae | TGT | | AACTTCGT | |
| Neisseria | GGCGCAACACAGGATTA | 67 | TTCAGCGTGATGATGTC | 68 |
| gonorrhoeae | TTTTCC | | GATAGG | |
| Neisseria | CGGTACGTTGGGATTGT | 69 | CGTCTTTGGAAACCGCG | 70 |
| gonorrhoeae | GAAC | | ATTTT | |
| Neisseria | TTGCCGGATGGAAACCT | 71 | GCGAATCGCGCAATGTT | 72 |
| gonorrhoeae | GATT | | TTC | |
| Neisseria | GGAAAACATTGCGCGAT | 73 | CGCTCATTTCGTAGCTT | 74 |
| gonorrhoeae | TCG | | TGGAA | |
| Neisseria | ACGAAATGAGCGCGGTA | 75 | GATTGCCGGTTTTCAGG | 76 |
| gonorrhoeae | GAG | | TTCTG | |
| Neisseria | CTGCATTTACCGCACCTA | 77 | CCGCATATTTGTCCGGT | 78 |
| gonorrhoeae | CAAAA | | TCGAT | |
| Neisseria | CTGCATTTACCGCACCTA | 79 | ACCAACTCTTCGTCCGC | 80 |
| gonorrhoeae | CAAAA | | ATATTT | |
| Neisseria | CCTCAACCGTGTCCGTA | 81 | CGCTTCCTGATGGGTTT | 82 |
| gonorrhoeae | AAGAG | | TATTGC | |
| Neisseria | CCTCAACCGTGTCCGTA | 83 | CGCTTCCTGATGGGTTT | 84 |
| gonorrhoeae | AAGAG | | TATTGC | |
| Neisseria | GCTTGGGCATTACGCAA | 85 | TCAGCGCAATCCCTATG | 86 |
| gonorrhoeae | TAAAAC | | ACTTC | |
| Trichomonas | CCCAGAAGCTCGTGAAG | 87 | CGCCACCTTCGACTGAT | 88 |
| vaginalis | ATCTTG | | TCA | |
| Trichomonas | CCCAGAAGCTCGTGAAG | 89 | CGCCACCTTCGACTGAT | 90 |
| vaginalis | ATCTTG | | TCA | |
| Trichomonas | CCGTACAACAGGTATCG | 91 | GGGAGTAGCCTTCGTAA | 92 |
| vaginalis | TTTTCGA | | ATTGGAA | |
| Trichomonas | ACAGTTCCAATTTACGA | 93 | CACGGCCAGCGAGGTT | 94 |
| vaginalis | AGGCTACT | | A | |
| Trichomonas | CGCTGGCCGTGATCTCA | 95 | GGCTGTTGTGTTGAAAG | 96 |
| vaginalis | | | CATTG | |
| Trichomonas | CTGGATGGTCAAGCTTC | 97 | CTTGATGTCACGAACGA | 98 |
| vaginalis | TCACA | | TTTCCTTT | |
| Trichomonas | CCGTACAACAGGTATCG | 99 | GGGAGTAGCCTTCGTAA | 100 |
| vaginalis | TTTTCGA | | ATTGGAA | |
| Trichomonas | ACAGTTCCAATTTACGA | 101 | GCCAGCGAGGTTAAGT | 102 |
| vaginalis | AGGCTACT | | CTCAT | |
| Trichomonas | TGATCTCACAGCCTGGA | 103 | GCTGTTGTGTTGAAAGC | 104 |
| vaginalis | TGGT | | ATTGC | |
| Trichomonas | GGATGGTCAAGCTTCTC | 105 | CTTGATGTCACGAACGA | 106 |
| vaginalis | ACAGA | | TTTCCTTT | |
| Human | AGATTTGGACCTGCGAG | 107 | GAGCGGCTGTCTCCACA | 108 |
| | CG | | AGT | |

TABLE 3
Probes and their SEQ ID NOs for detecting the STI pathogens in the 5-plex multiplex
assay.

| Organism name | Probe | SEQ ID NO: |
|---|---|---|
| Chlamydia trachomatis | TAGCAGGTCCGTCATTTC | 109 |
| Chlamydia trachomatis | ACGACAGACAAAGACTC | 110 |
| Chlamydia trachomatis | CAGCTCCGTCAGAGTCT | 111 |
| Chlamydia trachomatis | TCCGACAGAGGCATTC | 112 |
| Chlamydia trachomatis | CAGGTCCGTCATTTCC | 113 |
| Chlamydia trachomatis | TCCTACACAACAGGGCCTCGTAACG | 114 |
| (all serovars) | | |
| Chlamydia trachomatis | ACGATGGCATTTCCTTCTCCAATGAAACT | 115 |
| (all serovars) | | |
| Chlamydia trachomatis | ACCAACAACCAAACTGATGATATCAATACGACAG | 116 |
| (all serovars) | A | |
| Chlamydia trachomatis | CAAGATAGCAGAGCCCTACAAGAAACTGT | 117 |
| (all serovars) | | |
| Chlamydia trachomatis | TCAAGATAGCAGAGCCCTACAAGAAACTG | 118 |
| (all serovars) | | |
| Chlamydia trachomatis | CTGCTGCATTCATCCGAGCATCTTGAAGA | 119 |
| (all serovars) | | |
| Chlamydia trachomatis | ACGCTTCTTGCTTATATTCAACCGGACGC | 120 |
| (all serovars) | | |
| Chlamydia trachomatis | TAGCGAGAAGCGACATCTTTACGTTCGC | 121 |
| (all serovars) | | |
| Chlamydia trachomatis | TTGAGTGAGCTCCCCTTCTAAAAGAGCTC | 122 |
| (all serovars) | | |
| Chlamydia trachomatis sv | CTGCCGAAACTAGGAATATCGTAGCCATCACT | 123 |
| L (LGV specific) | | |
| Chlamydia trachomatis sv | CTGCCGAAACTAGGAATATCGTAGCCATCA | 124 |
| L (LGV specific) | | |
| Chlamydia trachomatis sv | TCTGACAGGGAATCGAACTCTCGCCTT | 125 |
| L (LGV specific) | | |
| Chlamydia trachomatis sv | TCGGATGTCAATGTAACCACAGTCAATAGCT | 126 |
| L (LGV specific) | | |
| Mycoplasma genitalium | TCCAAATTGACAGTGCAACAAACTGTTCTTGT | 127 |
| Mycoplasma genitalium | AAATGATCCAAATTGACAGTGCAACAAACTGT | 128 |
| Mycoplasma genitalium | TCTACCGTCCCCTAATGAAATACTGAAAAGATGA | 129 |
| Mycoplasma genitalium | TTGCCCTCTGTAGACAAGACCATATCCCT | 130 |
| Mycoplasma genitalium | CAGCCCCTTAAGCTTG | 131 |
| Mycoplasma genitalium | TTGTTTAGCCCAATTGCC | 132 |
| Mycoplasma genitalium | AACACCAGATCAACCTCA | 133 |
| Mycoplasma genitalium | CAACACAAGCAATCTTCT | 134 |
| Neisseria gonorrhoeae | TAGCGGTTTGCTGTAATAC | 135 |
| Neisseria gonorrhoeae | ACTGGCAGGACGGATAT | 136 |
| Neisseria gonorrhoeae | ACGCAAGCAAGAACG | 137 |
| Neisseria gonorrhoeae | CCCATACAGTCCGATATGC | 138 |
| Neisseria gonorrhoeae | CAGCAAACCGCTATCTG | 139 |
| Neisseria gonorrhoeae | ACGGTTGTCTATATCCGTCCTGCCAGT | 140 |
| Neisseria gonorrhoeae | CCTGCCAGTACGCAAGCAAGAACG | 141 |
| Neisseria gonorrhoeae | TCGTTTGCCGCCGCCATATCCA | 142 |
| Neisseria gonorrhoeae | CAGCAAGCCGCGCTCGACAA | 143 |
| Neisseria gonorrhoeae | CATAGACGGACGCGCCGCAATCAC | 144 |
| Neisseria gonorrhoeae | ACTGCCTTGCCCTGCTTCAGCG | 145 |
| Neisseria gonorrhoeae | CATGGCGGCTGAGTGCAATATCCCC | 146 |
| Neisseria gonorrhoeae | CCCATTGCCTGCGGCCCAT | 147 |
| Neisseria gonorrhoeae | CCTGCGGCCCATATCGAACCG | 148 |
| Neisseria gonorrhoeae | CCCAAGCGGTGTAGCAGCCCT | 149 |
| Neisseria gonorrhoeae | CTGCTACACCGCTTGGGCATTAC | 150 |
| Neisseria gonorrhoeae | CCATCAGGAAGCGCCGTGGATAC | 151 |
| Trichomonas vaginalis | ACTTCGTCGTAGTCCTTCTC | 152 |
| Trichomonas vaginalis | CTCCTCGAGAAGGACTACGACGAAGTT | 153 |
| Trichomonas vaginalis | CTGTGTGGGAAACACCATCACCAGC | 154 |
| Trichomonas vaginalis | CCCTTCCACACGCCATCATGAGACT | 155 |
| Trichomonas vaginalis | CACGCTCTGTGAGAAGCTTGACCAT | 156 |
| Trichomonas vaginalis | CTGTTGTGTTGAAAGCATTGCCACGCTC | 157 |
| Trichomonas vaginalis | CTGTGTGGGAAACACC | 158 |
| Trichomonas vaginalis | CCCTTCCACACGCCATC | 159 |
| Trichomonas vaginalis | AAGCTTCTCACAGAGCGTG | 160 |
| Trichomonas vaginalis | CTGTTGTGTTGAAAGCATT | 161 |
| Human | TTCTGACCTGAAGGCTCTGCGCG | 162 |

TABLE 4
Dye assignment for targeted STI pathogens

| Dyes | Quencher | Targeted STI Pathogen |
|---|---|---|
| FAM | MGB | Chlamydia trachomatis (CT) |
| VIC | MGB | Neisseria gonorrhoeae (NG) |
| ABY | MGB | Mycoplasma genitalium (MG) |
| JUN | MGB | Trichomonas vaginalis (TV) |
| AF647 | QSY21 | RNase P (Internal Control) |

TABLE 5
Primer and probe concentration for the 5-plex multiplex assay.

| | | Primer | Probe |
|---|---|---|---|
| Target | Dye | concentration | concentration |
| Chlamydia trachomatis | FAM | 300 nM | 250 nM |
| Neisseria gonorrhoeae | VIC | 300 nM | 250 nM |
| Mycoplasma genitalium | ABY | 300 nM | 250 nM |
| Trichomonas vaginalis | JUN | 300 nM | 250 nM |
| RNase P | AF647 | 300 nM | 150 nM |

TABLE 6
Strain coverage of the 5-plex multiplex assay for select genes.

| Organism name | Gene name | NCBI Strain coverage |
|---|---|---|
| Mycoplasma | ComEC/Rec2-related protein | 100% 6 out of 6 |
| genitalium | ComEC/Rec2-related protein | 100% 6 out of 6 |
| | tRNA(Ile)-lysidine synthetase | 100% 6 out of 6 |
| | tRNA(Ile)-lysidine synthetase | 100% 6 out of 6 |
| Trichomonas | Actin | 100% 132/132 |
| vaginalis | Actin | 99.24% 131/132 |
| | Actin | 99.24% 131/132 |
| | Actin | 97.72% 129/132 |
| Chlamydia | tarP | 100% 452/452 |
| trachomatis | tarP | 100% 452/452 |
| | tarP | 98.47% 452/465 |
| | tarP | 99.78% 465/466 |
| Neisseria | Restriction endonuclease, | 99.9% 1084/1085 |
| gonorrhoeae | type I, HsdS, NGO0357 | |
| | Restriction endonuclease, | 99.9% 1084/1085 |
| | type I, HsdS, NGO0357 | |
| | Restriction endonuclease, | 100% 1086/1086 |
| | type I, HsdS, NGO0357 | |
| | Restriction endonuclease, | 100% 1086/1086 |
| | type I, HsdS, NGO0357 | |

TABLE 7
Amplicons from target regions and their SEQ ID NOs for detecting the STI pathogens in
the 5-plex multiplex assays.

| | | SEQ | | | | |
|---|---|---|---|---|---|---|
| Organism | | ID | Size | Genbank | Position | Gene |
| name | Amplicon | NO: | (ntd) | Accession | targeted | name |
| Chlamydia | TCTGGAGATGATTCAGG | 163 | 101 | HM543594.1 | 1588..1646.. | tarP |
| trachomatis | AAGTGTCTCTTCCTCAG | | | | 1688 | |
| | AATCAGACAAGAATGC | | | | | |
| | CTCTGTCGGAAATGACG | | | | | |
| | GACCTGCTATGAAAGAT | | | | | |
| | ATCCTTTCTGCCGTGCG | | | | | |
| Chlamydia | CTCAATCTACAGACGCC | 164 | 135 | CP035484.1 | 867245.. | tarP |
| trachomatis | TCTACAGATACAAGCG | | | | 867379 | |
| | ATATCGATGACATAAAT | | | | | |
| | ACCAACAACCAAACTG | | | | | |
| | ATGATATCAATACGACA | | | | | |
| | GACAAAGACTCTGACG | | | | | |
| | GAGCTGGTGGAGTCAA | | | | | |
| | TGGCGATATATCCGAAA | | | | | |
| | CAG | | | | | |
| Chlamydia | CTCAATCTACAGACGCC | 165 | 135 | CP035484.1 | 867245.. | tarP |
| trachomatis | TCTACAGATACAAGCG | | | | 867379 | |
| | ATATCGATGACATAAAT | | | | | |
| | ACCAACAACCAAACTG | | | | | |
| | ATGATATCAATACGACA | | | | | |
| | GACAAAGACTCTGACG | | | | | |
| | GAGCTGGTGGAGTCAA | | | | | |
| | TGGCGATATATCCGAAA | | | | | |
| | CAG | | | | | |
| Chlamydia | TCTGGAGATGATTCAGG | 166 | 101 | CP035484.1 | 867388.. | tarP |
| trachomatis | AAGTGTCTCTTCCTCAG | | | | 867488 | |
| | AATCAGACAAGAATGC | | | | | |
| | CTCTGTCGGAAATGACG | | | | | |
| | GACCTGCTATGAAAGAT | | | | | |
| | ATCCTTTCTGCCGTGCG | | | | | |
| Chlamydia | TGGCGATATATCCGAAA | 167 | 129 | CP035484.1 | 867360.. | tarP |
| trachomatis | CAGAATCCTCTTCTGGA | | | | 867488 | |
| | GATGATTCAGGAAGTGT | | | | | |
| | CTCTTCCTCAGAATCAG | | | | | |
| | ACAAGAATGCCTCTGTC | | | | | |
| | GGAAATGACGGACCTG | | | | | |
| | CTATGAAAGATATCCTT | | | | | |
| | TCTGCCGTGCG | | | | | |
| Chlamydia | GCAGCAGCACTCAATTC | 168 | 77 | CP035484.1 | 866455.. | tarP |
| trachomatis | TCTAAGAGGCTCCTCCT | | | | 866531 | |
| (all | ACACAACAGGGCCTCG | | | | | |
| serovars) | TAACGAGGGTGTATTCG | | | | | |
| | GCCCTGGACC | | | | | |
| Chlamydia | CTCAAGCCGGGCCTAGT | 169 | 98 | CP035484.1 | 867056... | tarP |
| trachomatis | AGCGAAGACGATGGCA | | | | 867153 | |
| (all | TTTCCTTCTCCAATGAA | | | | | |
| serovars) | ACTCCTGGAGCTGGTCC | | | | | |
| | TGCAGCTGCTCCATCAC | | | | | |
| | CAACGCCATCCTCT | | | | | |
| Chlamydia | CGCCTCTACAGATACAA | 170 | 93 | CP035484.1 | 867258.. | tarP |
| trachomatis | GCGATATCGATGACATA | | | | 867350 | |
| (all | AATACCAACAACCAAA | | | | | |
| serovars) | CTGATGATATCAATACG | | | | | |
| | ACAGACAAAGACTCTG | | | | | |
| | ACGGAGCTGG | | | | | |
| Chlamydia | CAGAACATGCTCTTCAT | 171 | 76 | CP035484.1 | 952046.. | Outer |
| trachomatis | CCTAAAGCACAGTTTCT | | | | 952121 | membrane |
| (all | TGTAGGGCTCTGCTATC | | | | | protein |
| serovars) | TTGAAATGGGCCATCCT | | | | | assembly |
| | GACTTAGC | | | | | factor |
| | | | | | | BamD |
| Chlamydia | CAGAACATGCTCTTCAT | 172 | 76 | CP035484.1 | 952046.. | Outer |
| trachomatis | CCTAAAGCACAGTTTCT | | | | 952121 | membrane |
| (all | TGTAGGGCTCTGCTATC | | | | | protein |
| serovars) | TTGAAATGGGCCATCCT | | | | | assembly |
| | GACTTAGC | | | | | factor |
| | | | | | | BamD |
| Chlamydia | GCTTTGCAAGAGCCCTA | 173 | 89 | CP035484.1 | 952489.. | Outer |
| trachomatis | TAATGAACAGTATCTTC | | | | 952577 | membrane |
| (all | AAGATGCTCGGATGAA | | | | | protein |
| serovars) | TGCAGCAGCTTTACGTA | | | | | assembly |
| | AACAACACCCTAATCAT | | | | | factor |
| | CCTAG | | | | | BamD |
| Chlamydia | ACTATATTCATCACATG | 174 | 88 | CP035484.1 | 952595.. | Outer |
| trachomatis | TGCGAAGCTTACGCTTC | | | | 952682 | membrane |
| (all | TTGCTTATATTCAACCG | | | | | protein |
| serovars) | GACGCTTTTATGAGAAA | | | | | assembly |
| | AAGCGAAAAGCCTCTTC | | | | | factor |
| | TGC | | | | | BamD |
| Chlamydia | CAAGACCTTTTTTCGCT | 175 | 88 | CP017746.1 | 198045.. | Trp |
| trachomatis | GTTTTTGTCTTTTAGCG | | | | 198132 | operon |
| (all | AACGTAAAGATGTCGCT | | | | | repressor |
| serovars) | TCTCGCTATCATATCAT | | | | | |
| | TCGAGCTCTTTTAGAAG | | | | | |
| | GGG | | | | | |
| Chlamydia | GATGTCGCTTCTCGCTA | 176 | 85 | CP035484.1 | 527249.. | Trp |
| trachomatis | TCATATCATTCGAGCTC | | | | 527333 | operon |
| (all | TTTTAGAAGGGGAGCTC | | | | | repressor |
| serovars) | ACTCAAAGAGAGATAG | | | | | |
| | CAGAGAAATACGGAGT | | | | | |
| | CA | | | | | |
| Chlamydia | GGTGGGATTAGTGGTG | 177 | 100 | CP019387.1 | 831667.. | cysteine- |
| trachomatis | ATAGGACTACTTTTAGT | | | | 831568 | rich outer |
| sv L (LGV | GATGGCTACGATATTCC | | | | | membrane |
| specific) | TAGTTTCGGCAGCTTCG | | | | | protein |
| | TTTGTAAATCCCATCTA | | | | | (CRPA) |
| | TCTAGCTATTCCTGCT | | | | | |
| Chlamydia | GGTGGGATTAGTGGTG | 178 | 100 | CP019387.1 | 831667.. | cysteine- |
| trachomatis | ATAGGACTACTTTTAGT | | | | 831568 | rich outer |
| sv L (LGV | GATGGCTACGATATTCC | | | | | membrane |
| specific) | TAGTTTCGGCAGCTTCG | | | | | protein |
| | TTTGTAAATCCCATCTA | | | | | (CRPA) |
| | TCTAGCTATTCCTGCT | | | | | |
| Chlamydia | GGTGGTGCGCTTTATGC | 179 | 83 | CP019387.1 | 310792.. | polymorp |
| trachomatis | AAAGAATATCACTCTGA | | | | 310874 | hic outer |
| sv L (LGV | CAGGGAATCGAACTCTC | | | | | membrane |
| specific) | GCCTTTATTAACAATAT | | | | | protein |
| | GTCTGGAGACTGCGG | | | | | repeat- |
| | | | | | | containing |
| | | | | | | protein |
| Chlamydia | GATAGCAATACCGCTGC | 180 | 103 | CP019387.1 | 310313.. | polymorp |
| trachomatis | TCATGCCGGGGGAGCT | | | | 310415 | hic outer |
| sv L (LGV | ATTGACTGTGGTTACAT | | | | | membrane |
| specific) | TGACATCCGAAATAAC | | | | | protein |
| | GGTCCTATCTATTTTCT | | | | | repeat- |
| | AAATAACTCTGCTGCCT | | | | | containing |
| | GGG | | | | | protein |
| Mycoplasma | TTTTACTCAGTTTTATCC | 181 | 105 | CP003773.1 | 395267.. | ComEC/R |
| genitalium | CAGGCTTCTTTTTAGTA | | | | 395163 | ec2- |
| | CAACAAGAACAGTTTGT | | | | | related |
| | TGCACTGTCAATTTGGA | | | | | protein |
| | TCATTTTGATAACGCTT | | | | | |
| | TTTAGTCTTTGGTATGA | | | | | |
| | CT | | | | | |
| Mycoplasma | TTTTACTCAGTTTTATCC | 182 | 105 | CP003773.1 | 395267.. | ComEC/R |
| genitalium | CAGGCTTCTTTTTAGTA | | | | 395163 | ec2- |
| | CAACAAGAACAGTTTGT | | | | | related |
| | TGCACTGTCAATTTGGA | | | | | protein |
| | TCATTTTGATAACGCTT | | | | | |
| | TTTAGTCTTTGGTATGA | | | | | |
| | CT | | | | | |
| Mycoplasma | CCAGGTGTGAATTGTAT | 183 | 86 | CP003773.1 | 111295.. | tRNA(Ile)- |
| genitalium | GGTTTGAAAATCTACCG | | | | 111380 | lysidine |
| | TCCCCTAATGAAATACT | | | | | synthetase |
| | GAAAAGATGAAATCAT | | | | | |
| | TGCCCTCTGTAGACAAG | | | | | |
| | AC | | | | | |
| Mycoplasma | CCGTCCCCTAATGAAAT | 184 | 118 | CP003773.1 | 111326.. | tRNA(Ile)- |
| genitalium | ACTGAAAAGATGAAAT | | | | 111443 | lysidine |
| | CATTGCCCTCTGTAGAC | | | | | synthetase |
| | AAGACCATATCCCTTAT | | | | | |
| | GAGATTGATGAAACTA | | | | | |
| | ACAAGTTACCTATTTAT | | | | | |
| | AAGCGCAATGAAGTGA | | | | | |
| | GG | | | | | |
| Mycoplasma | GCTTTAAAACCGCTTGT | 185 | 123 | CP003773.1 | 394474.. | ComEC/R |
| genitalium | CAGTTCTAGTTTAATCT | | | | 394596 | ec2- |
| | TGATTGTTATCAGCCCC | | | | | related |
| | TTAAGCTTGTATTTAAA | | | | | protein |
| | TTCAAGACTAAATGCTT | | | | | |
| | TTAGTGTCTTGTTTAAC | | | | | |
| | CTTTTGTTTAGCCCAAT | | | | | |
| | TGCC | | | | | |
| Mycoplasma | ATTGTTATCAGCCCCTT | 186 | 145 | CP003773.1 | 394416.. | ComEC/R |
| genitalium | AAGCTTGTATTTAAATT | | | | 394560 | ec2- |
| | CAAGACTAAATGCTTTT | | | | | related |
| | AGTGTCTTGTTTAACCT | | | | | protein |
| | TTTGTTTAGCCCAATTG | | | | | |
| | CCTTATTTTATTTTTGTG | | | | | |
| | TTAGTTGAATTATTTTA | | | | | |
| | CCCTTTATTGGTGTTTTT | | | | | |
| | GGTCAAG | | | | | |
| Mycoplasma | GAAGATTGCTTGTGTTG | 187 | 118 | CP003773.1 | 110987.. | tRNA(Ile)- |
| genitalium | TTCATGTCAATTACAAC | | | | 111104 | lysidine |
| | ACCAGATCAACCTCATT | | | | | synthetase |
| | ACGTGATCAAAAGTTA | | | | | |
| | GTAGAACAATATTGTCA | | | | | |
| | AAAATTAAATATTCCAC | | | | | |
| | TGGTTGTTCACACTGTT | | | | | |
| Mycoplasma | GGTGGATCTGATTCAAT | 188 | 97 | CP003773.1 | 110946.. | tRNA(Ile)- |
| genitalium | GCTAATGCTTAAACTTT | | | | 111042 | lysidine |
| | ACCAAAAGAAGATTGC | | | | | synthetase |
| | TTGTGTTGTTCATGTCA | | | | | |
| | ATTACAACACCAGATCA | | | | | |
| | ACCTCATTACGTG | | | | | |
| Neisseria | CCATACAGTCCGATATG | 189 | 98 | NC_002946.2 | 348948.. | NGO0357 |
| gonorrhoeae | CCAATAGAAGTAAAAC | | | | 348996.. | |
| | TGTATTACGCTAAATGT | | | | 349045 | |
| | ATTACAGCAAACCGCTA | | | | | |
| | TCTGTTTTCTTATGGAC | | | | | |
| | GTAAACCCAAAGGC | | | | | |
| Neisseria | GCATTGCCGCAACCAA | 190 | 105 | AP019853.1 | 348324.. | NGO_0357 |
| gonorrhoeae | ATTAAAAGAATTTGAAC | | | | 348428 | hypothetical |
| | AACGGCAAAAAGACAC | | | | | protein |
| | GGTTGTCTATATCCGTC | | | | | |
| | CTGCCAGTACGCAAGC | | | | | |
| | AAGAACGCTACGAAGT | | | | | |
| | TATATCG | | | | | |
| Neisseria | CATTGCCGCAACCAAAT | 191 | 115 | AP019853.1 | 348325.. | NGO_0357 |
| gonorrhoeae | TAAAAGAATTTGAACA | | | | 348439 | hypothetical |
| | ACGGCAAAAAGACACG | | | | | protein |
| | GTTGTCTATATCCGTCC | | | | | |
| | TGCCAGTACGCAAGCA | | | | | |
| | AGAACGCTACGAAGTT | | | | | |
| | ATATCGCTCGTGATAGC | | | | | |
| Neisseria | GTAGCAAATAGTGATGT | 192 | 129 | AP019853.1 | 348536.. | NGO_0357 |
| gonorrhoeae | CGCCGTATTAACACCCA | | | | 348664 | hypothetical |
| | TACAGTCCGATATGCCA | | | | | protein |
| | ATAGAAGTAAAACTGT | | | | | |
| | ATTACGCTAAATGTATT | | | | | |
| | ACAGCAAACCGCTATCT | | | | | |
| | GTTTTCTTATGGACGTA | | | | | |
| | AACCCAAAGGC | | | | | |
| Neisseria | GATGTCGCCGTATTAAC | 193 | 117 | AP019853.1 | 348548.. | NGO_0357 |
| gonorrhoeae | ACCCATACAGTCCGATA | | | | 348664 | hypothetical |
| | TGCCAATAGAAGTAAA | | | | | protein |
| | ACTGTATTACGCTAAAT | | | | | |
| | GTATTACAGCAAACCGC | | | | | |
| | TATCTGTTTTCTTATGG | | | | | |
| | ACGTAAACCCAAAGGC | | | | | |
| Neisseria | GCATTGCCGCAACCAA | 194 | 105 | AP019853.1 | 348324.. | NGO_0357 |
| gonorrhoeae | ATTAAAAGAATTTGAAC | | | | 348428 | hypothetical |
| | AACGGCAAAAAGACAC | | | | | protein |
| | GGTTGTCTATATCCGTC | | | | | |
| | CTGCCAGTACGCAAGC | | | | | |
| | AAGAACGCTACGAAGT | | | | | |
| | TATATCG | | | | | |
| Neisseria | CGGCAAAAAGACACGG | 195 | 81 | AP019853.1 | 348359.. | NGO_0357 |
| gonorrhoeae | TTGTCTATATCCGTCCT | | | | 348439 | hypothetical |
| | GCCAGTACGCAAGCAA | | | | | protein |
| | GAACGCTACGAAGTTAT | | | | | |
| | ATCGCTCGTGATAGC | | | | | |
| Neisseria | GGCGCAACACAGGATT | 196 | 78 | CP045832.1 | 1267574.. | NGO_1110 |
| gonorrhoeae | ATTTTCCGCGCTCTGCT | | | | 1267497 | phage |
| | GGATATGGCGGCGGCA | | | | | protein |
| | AACGAGCCTATCGACAT | | | | | |
| | CATCACGCTGAA | | | | | |
| Neisseria | CGGTACGTTGGGATTGT | 197 | 91 | CP045832.1 | 1267309.. | NGO_1110 |
| gonorrhoeae | GAACGACAGGTTTGTCG | | | | 1267399 | phage |
| | AGCGCGGCTTGCTGAA | | | | | protein |
| | GGCTTCGGCGGCGATTG | | | | | |
| | AAAAAATCGCGGTTTCC | | | | | |
| | AAAGACG | | | | | |
| Neisseria | TTGCCGGATGGAAACCT | 198 | 83 | CP045832.1 | 1267114.. | NGO_1110 |
| gonorrhoeae | GATTGTGATTGCGGCGC | | | | 1267032 | phage |
| | GTCCGTCTATGGGTAAA | | | | | protein |
| | ACGGTTTTGGCGGAAA | | | | | |
| | ACATTGCGCGATTCGC | | | | | |
| Neisseria | GGAAAACATTGCGCGA | 199 | 68 | CP045832.1 | 1267052.. | NGO_1110 |
| gonorrhoeae | TTCGCGCTGAAGCAGG | | | | 1266985 | phage |
| | GCAAGGCAGTTCATTTC | | | | | protein |
| | CAAAGCTACGAAATGA | | | | | |
| | GCG | | | | | |
| Neisseria | ACGAAATGAGCGCGGT | 200 | 84 | CP045832.1 | 1266996.. | NGO_1110 |
| gonorrhoeae | AGAGCTGGCAAGGCGC | | | | 1266913 | phage |
| | GGCATGGCGGCTGAGT | | | | | protein |
| | GCAATATCCCCATGCAG | | | | | |
| | AACCTGAAAACCGGCA | | | | | |
| | ATC | | | | | |
| Neisseria | CTGCATTTACCGCACCT | 201 | 77 | CP045832.1 | 1546149.. | NGO_1117 |
| gonorrhoeae | ACAAAAAATACGGCGG | | | | 1546073 | phage |
| | CAGCCCATTGCCTGCGG | | | | | protein |
| | CCCATATCGAACCGGAC | | | | | |
| | AAATATGCGG | | | | | |
| Neisseria | CTGCATTTACCGCACCT | 202 | 90 | CP045832.1 | 1546149.. | NGO_1117 |
| gonorrhoeae | ACAAAAAATACGGCGG | | | | 1546060 | phage |
| | CAGCCCATTGCCTGCGG | | | | | protein |
| | CCCATATCGAACCGGAC | | | | | |
| | AAATATGCGGACGAAG | | | | | |
| | AGTTGGT | | | | | |
| Neisseria | CCTCAACCGTGTCCGTA | 203 | 73 | CP045832.1 | 1546056.. | NGO_1117 |
| gonorrhoea | AAGAGCAGGGCTGCTA | | | | 1545984 | phage |
| e | CACCGCTTGGGCATTAC | | | | | protein |
| | GCAATAAAACCCATCA | | | | | |
| | GGAAGCG | | | | | |
| Neisseria | CCTCAACCGTGTCCGTA | 204 | 73 | CP045832.1 | 1546056.. | NGO_1117 |
| gonorrhoeae | AAGAGCAGGGCTGCTA | | | | 1545984 | phage |
| | CACCGCTTGGGCATTAC | | | | | protein |
| | GCAATAAAACCCATCA | | | | | |
| | GGAAGCG | | | | | |
| Neisseria | GCTTGGGCATTACGCAA | 205 | 82 | CP045832.1 | 1546019.. | NGO_1117 |
| gonorrhoeae | TAAAACCCATCAGGAA | | | | 1545938 | phage |
| | GCGCCGTGGATACAGA | | | | | protein |
| | CCCGGCAGGGCGAAGT | | | | | |
| | CATAGGGATTGCGCTGA | | | | | |
| Trichomonas | CCCAGAAGCTCGTGAA | 206 | 77 | NW_ | 5857..5888.. | α-TUB1 |
| vaginalis | GATCTTGCCCTCCTCGA | | | 001580342.1 | 5933 | |
| | GAAGGACTACGACGAA | | | | | |
| | GTTGCTGCTGAATCAGT | | | | | |
| | CGAAGGTGGCG | | | | | |
| Trichomonas | CCCAGAAGCTCGTGAA | 207 | 77 | DS114717.1 | 5857..5882.. | α-TUB1 |
| vaginalis | GATCTTGCCCTCCTCGA | | | | 5933 | |
| | GAAGGACTACGACGAA | | | | | |
| | GTTGCTGCTGAATCAGT | | | | | |
| | CGAAGGTGGCG | | | | | |
| Trichomonas | CCGTACAACAGGTATCG | 208 | 74 | MG907517.1 | 195..268 | actin |
| vaginalis | TTTTCGATGCTGGTGAT | | | | | |
| | GGTGTTTCCCACACAGT | | | | | |
| | TCCAATTTACGAAGGCT | | | | | |
| | ACTCCC | | | | | |
| Trichomonas | ACAGTTCCAATTTACGA | 209 | 67 | MG907517.1 | 241..307 | actin |
| vaginalis | AGGCTACTCCCTTCCAC | | | | | |
| | ACGCCATCATGAGACTT | | | | | |
| | AACCTCGCTGGCCGTG | | | | | |
| Trichomonas | CGCTGGCCGTGATCTCA | 210 | 73 | MG907517.1 | 297..369 | actin |
| vaginalis | CAGCCTGGATGGTCAA | | | | | |
| | GCTTCTCACAGAGCGTG | | | | | |
| | GCAATGCTTTCAACACA | | | | | |
| | ACAGCC | | | | | |
| Trichomonas | CTGGATGGTCAAGCTTC | 211 | 79 | MG907517.1 | 318..396 | actin |
| vaginalis | TCACAGAGCGTGGCAA | | | | | |
| | TGCTTTCAACACAACAG | | | | | |
| | CCGAAAAGGAAATCGT | | | | | |
| | TCGTGACATCAAG | | | | | |
| Trichomonas | CCGTACAACAGGTATCG | 212 | 74 | MG907517.1 | 195..268 | actin |
| vaginalis | TTTTCGATGCTGGTGAT | | | | | |
| | GGTGTTTCCCACACAGT | | | | | |
| | TCCAATTTACGAAGGCT | | | | | |
| | ACTCCC | | | | | |
| Trichomonas | ACAGTTCCAATTTACGA | 213 | 63 | MG907517.1 | 241..303 | actin |
| vaginalis | AGGCTACTCCCTTCCAC | | | | | |
| | ACGCCATCATGAGACTT | | | | | |
| | AACCTCGCTGGC | | | | | |
| Trichomonas | TGATCTCACAGCCTGGA | 214 | 63 | MG907517.1 | 306..368 | actin |
| vaginalis | TGGTCAAGCTTCTCACA | | | | | |
| | GAGCGTGGCAATGCTTT | | | | | |
| | CAACACAACAGC | | | | | |
| Trichomonas | GGATGGTCAAGCTTCTC | 215 | 77 | MG907517.1 | 320..396 | actin |
| vaginalis | ACAGAGCGTGGCAATG | | | | | |
| | CTTTCAACACAACAGCC | | | | | |
| | GAAAAGGAAATCGTTC | | | | | |
| | GTGACATCAAG | | | | | |
| Human | AGATTTGGACCTGCGAG | 216 | 65 | | | RNase P |
| | CGGGTTCTGACCTGAAG | | | | | CDC |
| | GCTCTGCGCGGACTTGT | | | | | |
| | GGAGACAGCCGCTC | | | | | |

TABLE 8
Amplicon sequences with 25 nucleotide flanking regions and SEQ ID NOs.

| | | SEQ |
|---|---|---|
| Organism | | ID |
| name | 25 ntflan + amplicon + 25 ntflank | NO: |
| Chlamydia | CGATATATCCGAAACAGAATCCTCTTCTGGAGATGATTCAGGAAGTG | 217 |
| trachomatis | TCTCTTCCTCAGAATCAGACAAGAATGCCTCTGTCGGAAATGACGGA | |
| | CCTGCTATGAAAGATATCCTTTCTGCCGTGCGTAAACACCTAGACGT | |
| | CGTTTACCCT | |
| Chlamydia | AACACGACTAACACCACACCAACAACTCAATCTACAGACGCCTCTA | 218 |
| trachomatis | CAGATACAAGCGATATCGATAACATAAATACCAACAACCAAACTGA | |
| | TGATATCAATACGACAGACAAAGACTCTGACGGAGCTGGTGGAGTC | |
| | AATGGCGATATATCCGAAACAGAATCCTCTTCTGGAGATGATTCAGG | |
| Chlamydia | same as SEQ ID NO: 218 | 219 |
| trachomatis | | |
| Chlamydia | CGATATATCCGAAACAGAATCCTCTTCTGGAGATGATTCAGGAAGTG | 220 |
| trachomatis | TCTCTTCCTCAGAATCAGACAAGAATGCCTCTGTCGGAAATGACGGA | |
| | CCTGCTATGAAAGATATCCTTTCTGCCGTGCGTAAACACCTAGACGT | |
| | CGTTTACCCT | |
| Chlamydia | ACTCTGACGGAGCTGGTGGAGTCAATGGCGATATATCCGAAACAGA | 221 |
| trachomatis | ATCCTCTTCTGGAGATGATTCAGGAAGTGTCTCTTCCTCAGAATCAG | |
| | ACAAGAATGCCTCTGTCGGAAATGACGGACCTGCTATGAAAGATAT | |
| | CCTTTCTGCCGTGCGTAAACACCTAGACGTCGTTTACCCT | |
| Chlamydia | CCCAGAAAATACGAGTGGTGGTGCAGCAGCAGCACTCAATTCTCTA | 222 |
| trachomatis | AGAGGCTCCTCCTACACAACAGGGCCTCGTAACGAGGGTGTATTCG | |
| (all serovars) | GCCCTGGACCGGAAGGACTACCAGACATGTCTCTT | |
| Chlamydia | GACTTTAATAAAATCAACTGGGGAACTCAAGCCGGGCCTAGTAGCG | 223 |
| trachomatis | AAGACGATGGCATTTCCTTCTCCAATGAAACTCCTGGAGCTGGTCCT | |
| (all serovars) | GCAGCTGCTCCATCACCAACGCCATCCTCTATTCCTGTCATCAATGT | |
| | CAATGTCA | |
| Chlamydia | CCACACCAACAACTCAATCTACAGACGCCTCTACAGATACAAGCGA | 224 |
| trachomatis | TATCGATAACATAAATACCAACAACCAAACTGATGATATCAATACG | |
| (all serovars) | ACAGACAAAGACTCTGACGGAGCTGGTGGAGTCAATGGCGATATAT | |
| | CCGAA | |
| Chlamydia | TTTGGAATGATAACACATCATTTCCCAGAACATGCTCTTCATCCTAA | 225 |
| trachomatis | AGCACAGTTTCTTGTAGGGCTCTGCTATCTTGAAATGGGCCATCCTG | |
| (all serovars) | ACTTAGCGGATAAAGCGCTAACTCAATATCAA | |
| Chlamydia | TTTGGAATGATAACACATCATTTCCCAGAACATGCTCTTCATCCTAA | 226 |
| trachomatis | AGCACAGTTTCTTGTAGGGCTCTGCTATCTTGAAATGGGCCATCCTG | |
| (all serovars) | ACTTAGCGGATAAAGCGCTAACTCAATATCAA | |
| Chlamydia | TATTGCAAAAATCCATTGCTTACAAGCTTTGCAAGAGCCCTATAATG | 227 |
| trachomatis | AACAGTATCTTCAAGATGCTCGGATGAATGCAGCAGCTTTACGTAAA | |
| (all serovars) | CAACACCCTAATCATCCTAGCAATACAGAAGTAGAGAACTATATT | |
| Chlamydia | CATCCTAGCAATACAGAAGTAGAGAACTATATTCATCACATGTGCG | 228 |
| trachomatis | AAGCTTACGCTTCTTGCTTATATTCAACCGGACGCTTTTATGAGAAA | |
| (all serovars) | AAGCGAAAAGCCTCTTCTGCAAAAATTTATTACTCAATAGCTCTA | |
| Chlamydia | TAAAATGCAAAAAGAAAAGTTTTTACAAGACCTTTTTTCGCTGTTTT | 229 |
| trachomatis | TGTCTTTTAGCGAACGTAAAGATGTCGCTTCTCGCTATCATATCATTC | |
| (all serovars) | GAGCTCTTTTAGAAGGGGAGCTCACTCAAAGAGAGATAGCAGA | |
| Chlamydia | GTTTTTGTCTTTTGGCGAACGTAAAGATGTCGCTTCTCGCTATCATAT | 230 |
| trachomatis | CATTCGAGCTCTTTTAGAAGGGGAGCTCACTCAAAGAGAGATAGCA | |
| (all serovars) | GAGAAATACGGAGTCAGTATCGCACAAATTACCAGAGGATC | |
| Chlamydia | CGCAGATATTCACGCATCCCACAATAGCAGGAATAGCTAGATAGAT | 231 |
| trachomatis | GGGATTTACAAACGAAGCTGCCGAAACTAGGAATATCGTAGCCATC | |
| SV L (LGV | ACTAAAAGTAGTCCTATCACCACTAATCCCACCTTAAATGCAGTGGA | |
| specific) | AGATAGAAGAT | |
| Chlamydia | CGCAGATATTCACGCATCCCACAATAGCAGGAATAGCTAGATAGAT | 232 |
| trachomatis | GGGATTTACAAACGAAGCTGCCGAAACTAGGAATATCGTAGCCATC | |
| sv L (LGV | ACTAAAAGTAGTCCTATCACCACTAATCCCACCTTAAATGCAGTGGA | |
| specific) | AGATAGAAGAT | |
| Chlamydia | TTGAGTGTTAGCGGCGATAGCTCCCCCGCAGTCTCCAGACATATTGT | 233 |
| trachomatis | TAATAAAGGCGAGAGTTCGATTCCCTGTCAGAGTGATATTCTTTGCA | |
| sv L (LGV | TAAAGCGCACCACCCCTATTTTTTTCGCTTTTATTTCCT | |
| specific) | | |
| Chlamydia | AGTTTCGATAAATTAAAGGCTGCTCCCCAGGCAGCAGAGTTATTTAG | 234 |
| trachomatis | AAAATAGATAGGACCGTTATTTCGGATGTCAATGTAACCACAGTCA | |
| Sv L (LGV | ATAGCTCCCCCGGCATGAGCAGCGGTATTGCTATCGAAATAGATAG | |
| specific) | GACCTTTATTGTTT | |
| Mycoplasma | GTTAAGTTCAATAAACAAAACTTTCAGTCATACCAAAGACTAAAAA | 235 |
| genitalium | GCGTTATCAAAATGATCCAAATTGACAGTGCAACAAACTGTTCTTGT | |
| | TGTACTAAAAAGAAGCCTGGGATAAAACTGAGTAAAACAAAAAAAT | |
| | AAAACAGCTTTGTTTG | |
| Mycoplasma | GTTAAGTTCAATAAACAAAACTTTCAGTCATACCAAAGACTAAAAA | 236 |
| genitalium | GCGTTATCAAAATGATCCAAATTGACAGTGCAACAAACTGTTCTTGT | |
| | TGTACTAAAAAGAAGCCTGGGATAAAACTGAGTAAAACAAAAAAAT | |
| | AAAACAGCTTTGTTTG | |
| Mycoplasma | CGTGCTGTTTACTATGGGATTAAAACCAGGTGTGAATTGTATGGTTT | 237 |
| genitalium | GAAAATCTACCGTCCCCTAATGAAATACTGAAAAGATGAAATCATT | |
| | GCCCTCTGTAGACAAGACCATATCCCTTATGAGATTGATGAAA | |
| Mycoplasma | GTGAATTGTATGGTTTGAAAATCTACCGTCCCCTAATGAAATACTGA | 238 |
| genitalium | AAAGATGAAATCATTGCCCTCTGTAGACAAGACCATATCCCTTATGA | |
| | GATTGATGAAACTAACAAGTTACCTATTTATAAGCGCAATGAAGTG | |
| | AGGTTAGAGATAGAAAAATGGTCTAAAA | |
| Mycoplasma | TTCAACTAACACAAAAATAAAATAAGGCAATTGGGCTAAACAAAAG | 239 |
| genitalium | GTTAAACAAGACACTAAAAGCATTTAGTCTTGAATTTAAATACAAGC | |
| | TTAAGGGGCTGATAACAATCAAGATTAAACTAGAACTGACAAGCGG | |
| | TTTTAAAGCTTTTAATAACTTTAGTTTGTTGACA | |
| Mycoplasma | AGTGGGAGATAAATACCAAAACTAGCTTGACCAAAAACACCAATAA | 240 |
| genitalium | AGGGTAAAATAATTCAACTAACACAAAAATAAAATAAGGCAATTGG | |
| | GCTAAACAAAAGGTTAAACAAGACACTAAAAGCATTTAGTCTTGAA | |
| | TTTAAATACAAGCTTAAGGGGCTGATAACAATCAAGATTAAACTAG | |
| | AACTGACAAGC | |
| Mycoplasma | TGCTAATGCTTAAACTTTACCAAAAGAAGATTGCTTGTGTTGTTCAT | 241 |
| genitalium | GTCAATTACAACACCAGATCAACCTCATTACGTGATCAAAAGTTAGT | |
| | AGAACAATACTGTCAAAAATTAAATATTCCACTGGTTGTTCACACTG | |
| | TTGATCCTGATCTAGTTTGAAAGAAGA | |
| Mycoplasma | AAAACAATATATAGCAGGGGTTTCAGGTGGATCTGATTCAATGCTA | 242 |
| genitalium | ATGCTTAAACTTTACCAAAAGAAGATTGCTTGTGTTGTTCATGTCAA | |
| | TTACAACACCAGATCAACCTCATTACGTGATCAAAAGTTAGTAGAAC | |
| | AATACTG | |
| Neisseria | AATAGTGATGTCGCCGTATTAACACCCATACAGTCCGATATGCCAAT | 243 |
| gonorrhoeae | AGAAGTAAAACTGTATTACGCTAAATGTATTACAGCAAACCGCTATC | |
| | TGTTTTCTTATGGACGTAAACCCAAAGGCGAAAAATTAAAAAGCATT | |
| | ATGTTAC | |
| Neisseria | same as SEQ ID NO: 248 | 244 |
| gonorrhoeae | | |
| Neisseria | ATGAAATTTTTACTATTCAAAACGGCATTGCCGCAACCAAATTAAAA | 245 |
| gonorrhoeae | GAATTTGAACAACGGCAAAAAGACACGGTTGTCTATATCCGTCCTGC | |
| | CAGTACGCAAGCAAGAACGCTACGAAGTTATATCGCTCGTGATAGC | |
| | GTTGATGAAAAACATATTTTTCCAT | |
| Neisseria | TAGCTATGTTTCAACTTGTGAATTTGTAGCAAATAGTGATGTCGCCG | 246 |
| gonorrhoeae | TATTAACACCCATACAGTCCGATATGCCAATAGAAGTAAAACTGTAT | |
| | TACGCTAAATGTATTACAGCAAACCGCTATCTGTTTTCTTATGGACG | |
| | TAAACCCAAAGGCGAAAAATTAAAAAGCATTATGTTAC | |
| Neisseria | same as SEQ ID NO: 246 | 247 |
| gonorrhoeae | | |
| Neisseria | GATGAAATTTTTACTATTCAAAACGGCATTGCCGCAACCAAATTAAA | 248 |
| gonorrhoeae | AGAATTTGAACAACGGCAAAAAGACACGGTTGTCTATATCCGTCCT | |
| | GCCAGTACGCAAGCAAGAACGCTACGAAGTTATATCGCTCGTGATA | |
| | GCGTTGATGAAAAACA | |
| Neisseria | AACCAAATTAAAAGAATTTGAACAACGGCAAAAAGACACGGTTGTC | 249 |
| gonorrhoeae | TATATCCGTCCTGCCAGTACGCAAGCAAGAACGCTACGAAGTTATAT | |
| | CGCTCGTGATAGCGTTGATGAAAAACATATTTTTCCAT | |
| Neisseria | CTCGCCGCGCGCTTCCAGCTTGTCGTTCAGCGTGATGATGTCGATAG | 250 |
| gonorrhoeae | GCTCGTTTGCCGCCGCCATATCCAGCAGAGCGCGGAAAATAATCCTG | |
| | TGTTGCGCCTGGTAAAACTTTTCAGGGGTCAGGA | |
| Neisseria | GACAGCTTTTCTGCGACCGTCCCGCCGTCTTTGGAAACCGCGATTTT | 251 |
| gonorrhoeae | TTCAATCGCCGCCGAAGCCTTCAGCAAGCCGCGCTCGACAAACCTGT | |
| | CGTTCACAATCCCAACGTACCGGCTGATATTCTTCGCGCTTGGGGTG | |
| Neisseria | ATGAACTGCCTTGCCCTGCTTCAGCGCGAATCGCGCAATGTTTTCCG | 252 |
| gonorrhoeae | CCAAAACCGTTTTACCCATAGACGGACGCGCCGCAATCACAATCAG | |
| | GTTTCCATCCGGCAAACCGCCGGTCATCCCGTCAAGCTTC | |
| Neisseria | ATGCCGCGCCTTGCCAGCTCTACCGCGCTCATTTCGTAGCTTTGGAA | 253 |
| gonorrhoeae | ATGAACTGCCTTGCCCTGCTTCAGCGCGAATCGCGCAATGTTTTCCG | |
| | CCAAAACCGTTTTACCCATAGACG | |
| Neisseria | ATATTGGCGTAATCGCTTTGCGTCAGATTGCCGGTTTTCAGGTTCTGC | 254 |
| gonorrhoeae | ATGGGGATATTGCACTCAGCCGCCATGCCGCGCCTTGCCAGCTCTAC | |
| | CGCGCTCATTTCGTAGCTTTGGAAATGAACTGCCTTGCC | |
| Neisseria | CGGTTGAGGACAACCAACTCTTCGTCCGCATATTTGTCCGGTTCGAT | 255 |
| gonorrhoeae | ATGGGCCGCAGGCAATGGGCTGCCGCCGTATTTTTTGTAGGTGCGGT | |
| | AAATGCAGGGGACTACCGGACCGTGCTGCCAGT | |
| Neisseria | CTCTTTACGGACACGGTTGAGGACAACCAACTCTTCGTCCGCATATT | 256 |
| gonorrhoeae | TGTCCGGTTCGATATGGGCCGCAGGCAATGGGCTGCCGCCGTATTTT | |
| | TTGTAGGTGCGGTAAATGCAGGGGACTACCGGACCGTGCTGCCAGT | |
| Neisseria | CGCCCTGCCGGGTCTGTATCCACGGCGCTTCCTGATGGGTTTTATTG | 257 |
| gonorrhoeae | CGTAATGCCCAAGCGGTGTAGCAGCCCTGCTCTTTACGGACACGGTT | |
| | GAGGACAACCAACTCTTCGTCCGCATATT | |
| Neisseria | CGCCCTGCCGGGTCTGTATCCACGGCGCTTCCTGATGGGTTTTATTG | 258 |
| gonorrhoeae | CGTAATGCCCAAGCGGTGTAGCAGCCCTGCTCTTTACGGACACGGTT | |
| | GAGGACAACCAACTCTTCGTCCGCATATT | |
| Neisseria | AGCGCATGGCGGAAATATTCCCCCATCAGCGCAATCCCTATGACTTC | 259 |
| gonorrhoeae | GCCCTGCCGGGTCTGTATCCACGGCGCTTCCTGATGGGTTTTATTGC | |
| | GTAATGCCCAAGCGGTGTAGCAGCCCTGCTCTTTACGG | |
| Trichomonas | GTGAAGGTATGGAAGAAGCTGAATTCCCAGAAGCTCGTGAAGATCT | 260 |
| vaginalis | TGCCCTCCTCGAGAAGGACTACGACGAAGTTGCTGCTGAATCAGTCG | |
| | AAGGTGGCGACGAAGAAGAAGATGGTGGCGAAAT | |
| Trichomonas | GTGAAGGTATGGAAGAAGCTGAATTCCCAGAAGCTCGTGAAGATCT | 261 |
| vaginalis | TGCCCTCCTCGAGAAGGACTACGACGAAGTTGCTGCTGAATCAGTCG | |
| | AAGGTGGCGACGAAGAAGAAGATGGTGGCGAAAT | |
| Trichomonas | CTGTTCTTTCCCTCTACTCCTCTGGCCGTACAACAGGTATCGTTTTCG | 262 |
| vaginalis | ATGCTGGTGATGGTGTTTCCCACACAGTTCCAATTTACGAAGGCTAC | |
| | TCCCTTCCACACGCCATCATGAGACTTAA | |
| Trichomonas | CGATGCTGGTGATGGTGTTTCCCACACAGTTCCAATTTACGAAGGCT | 263 |
| vaginalis | ACTCCCTTCCACACGCCATCATGAGACTTAACCTCGCTGGCCGTGAT | |
| | CTCACAGCCTGGATGGTCAAGCT | |
| Trichomonas | CACACGCCATCATGAGACTTAACCTCGCTGGCCGTGATCTCACAGCC | 264 |
| vaginalis | TGGATGGTCAAGCTTCTCACAGAGCGTGGCAATGCTTTCAACACAAC | |
| | AGCCGAAAAGGAAATCGTTCGTGACATCA | |
| Trichomonas | ACCTCGCTGGCCGTGATCTCACAGCCTGGATGGTCAAGCTTCTCACA | 265 |
| vaginalis | GAGCGTGGCAATGCTTTCAACACAACAGCCGAAAAGGAAATCGTTC | |
| | GTGACATCAAGGAGAAGCTTTGCTATGTCGCCCTCG | |
| Trichomonas | CTGTTCTTTCCCTCTACTCCTCTGGCCGTACAACAGGTATCGTTTTCG | 266 |
| vaginalis | ATGCTGGTGATGGTGTTTCCCACACAGTTCCAATTTACGAAGGCTAC | |
| | TCCCTTCCACACGCCATCATGAGACTTAA | |
| Trichomonas | CGATGCTGGTGATGGTGTTTCCCACACAGTTCCAATTTACGAAGGCT | 267 |
| vaginalis | ACTCCCTTCCACACGCCATCATGAGACTTAACCTCGCTGGCCGTGAT | |
| | CTCACAGCCTGGATGGTCA | |
| Trichomonas | TCATGAGACTTAACCTCGCTGGCCGTGATCTCACAGCCTGGATGGTC | 268 |
| vaginalis | AAGCTTCTCACAGAGCGTGGCAATGCTTTCAACACAACAGCCGAAA | |
| | AGGAAATCGTTCGTGACATC | |
| Trichomonas | CTCGCTGGCCGTGATCTCACAGCCTGGATGGTCAAGCTTCTCACAGA | 269 |
| vaginalis | GCGTGGCAATGCTTTCAACACAACAGCCGAAAAGGAAATCGTTCGT | |
| | GACATCAAGGAGAAGCTTTGCTATGTCGCCCTCG | |
| Human | GGGACTTCAGCATGGCGGTGTTTGCAGATTTGGACCTGCGAGCGGGT | 270 |
| | TCTGACCTGAAGGCTCTGCGCGGACTTGTGGAGACAGCCGCTCACCT | |
| | TGGCTATTCAGTTGTTGCTAT | |

TABLE 9
Insert sequence for Super plasmid: CT_STI PC amplification control.

| | SEQ ID |
|---|---|
| Insert Sequence | NO: |
| GGCCGTCAAGGCCAACACGACTAACACCACACCAACAACTCAATCT | 271 |
| ACAGACGCCTCTACAGATACAAGCGATATCGATAACATAAATACCA | |
| ACAACCAAACTGATGATATCAATACGACAGACAAAGACTCTGACGG | |
| AGCTGGTGGAGTCAATGGCGATATATCCGAAACAGAATCCTCTTCTG | |
| GAGATGATTCAGGAAGTGTCTCTTCCTCAGAATCAGACAAGAATGCC | |
| TCTGTCGGAAATGACGGACCTGCTATGAAAGATATCCTTTCTGCCGT | |
| GCGTAAACACCTAGACGTCGTTTACCCTAAAACAATATATAGCAGG | |
| GGTTTCAGGTGGATCTGATTCAATGCTAATGCTTAAACTTTACCAAA | |
| AGAAGATTGCTTGTGTTGTTCATGTCAATTACAACACCAGATCAACC | |
| TCATTACGTGATCAAAAGTTAGTAGAACAATACTGTCAAAAATTAAA | |
| TATTCCACTGGTTGTTCACACTGTTGATCCTGATCTAGTTTGAAAGAA | |
| GAATGAAATTTTTACTATTCAAAACGGCATTGCCGCAACCAAATTAA | |
| AAGAATTTGAACAACGGCAAAAAGACACGGTTGTCTATATCCGTCCT | |
| GCCAGTACGCAAGCAAGAACGCTACGAAGTTATATCGCTCGTGATA | |
| GCGTTGATGAAAAACATATTTTTCCATCTGTTCTTTCCCTCTACTCCT | |
| CTGGCCGTACAACAGGTATCGTTTTCGATGCTGGTGATGGTGTTTCC | |
| CACACAGTTCCAATTTACGAAGGCTACTCCCTTCCACACGCCATCAT | |
| GAGACTTAACCTCGCTGGCCGTGATCTCACAGCCTGGATGGTCAAGC | |
| TTCTCACAGAGCGTGGCAATGCTTTCAACACAACAGCCGAAAAGGA | |
| AATCGTTCGTGACATCAAGGAGAAGCTTTGCTATGTCGCCCTCGATG | |
| GCGGTGTTTGCAGATTTGGACCTGCGAGCGGGTTCTGACCTGAAGGC | |
| TCTGCGCGGACTTGTGGAGACAGCCGCTCACCGTGAGTTGCACAGAT | |
| CTAAGCGTGGAAGTGGCTATATAGAGCAGATATATTATATGACGTAC | |
| CAGAGGATCACCTACTAAAAGACTTTTCGAGAATCTACACCTACTAG | |
| CAAGGGTGCGGAGGGAAGCTCATCAGTGGGGCCACGAGCTGAGTGC | |
| GTCCTGTCACTCCACTCCCATGTCCCTTGGGAAGGTCTGAGACTAGG | |
| GACGCGTGGCCTCATGGGCC | |

### EXAMPLES

The following examples may reference specific target nucleic acids, compositions, formulations, and/or process steps. It will be understood, however, that these examples may be modified by using any of the components described elsewhere herein, including by using any of the primers and/or probes described herein.

### Example 1. Assay Panel Performance with DNA Fragments

### Materials and Methods

The following materials were used for the assays, and the following assay requirements were achieved.

TABLE 10
Materials and requirements of 5-plex multiplex
assay for sensitive detection of STI pathogens.

| Parameter | Requirement |
|---|---|
| Instrument | QS5, 96 wp, 0.2 mL |
| Sample Type | Genital swab |
| Sample Prep | Recommendation Agnostic to sample prep; |
| | MagMax Viral/Pathogen II (MVP II) Nucleic |
| | Acid Isolation Kit (RUO), MVP I/II/Ultra |
| | kit, MDPHD (CE-IVDR) |
| Form Factor | Multiplex (5-Plex) in a tube |
| Master Mix | TaqPath ™ Infectious Disease 1-Step |
| | Multiplex Master Mix (No ROX) |
| Controls | Amplification Control; Internal Control |
| Protocol | DNA & RNA inputs |
| Compatibility | |
| PCR Input Volume | 17.5 μL |
| Specificity | >95% (in silico) |
| Sensitivity | <100 copies/rxn |
| Linear Dynamic | Serial dilution in four orders of magnitude or greater |
| Range | |
| In-use stability | 1 h when stored at 2° C.-8° C. |
| Precision | Intermediate level: 2 operators/2 |
| | instruments/3 days (one |
| | representative strain per analyte) |
| Time to first result | <120 min from extracted sample |

According to several studies species are different when their Average Nucleotide Identity (ANI) is less than 96.5% (Varghese et al., Nucleic Acids Res. 2015, 43(14): 6761-6771). Finding a new assay target for Neisseria gonorrhoeae with >95% strain coverage was challenging: N. gonorrhoeae is 95% similar to Neisseria meningitidis and 93% similar to Neisseria lactamica. By comparing several genomes in abundance profiles, genes that exist in all N. gonorrhoeae strains were identified, but not in lactamica, meningitidis, elongata, sicca etc. The developed assay had 99.9% strain coverage without targeting any other species under Neisseria genus.

For sensitivity and non-specificity determination, a Cq cut-off of 37 for all pathogen targets was used. Best assays were selected in each phase using a quantitative approach by adopting assay scoring criteria noted in Table 11. Amplification curves were inspected to ensure there are no anomalies. To ensure assay fulfills benchtop stability of 1 hr, all runs during the assay screening and panel finalization were performed after incubation at 4° C. for a more stringent 2 hrs.

TABLE 11
Criteria and scoring for the 5-plex amplification.

| Criteria | Scoring |
|---|---|
| PCR efficiency | 1 - 90% ≤ E ≤ 110% |
| Linearity | 1 - R² ≥ 0.98 |
| Sensitivity at 10cp/rxn | 1 - 100% hit rate |
| Mean ΔRn at 10cp/rxn | 1 - >10\hat{ }5 |
| | 0.5 - >10\hat{ }4 |
| Non-specific amplification | 1 - No |

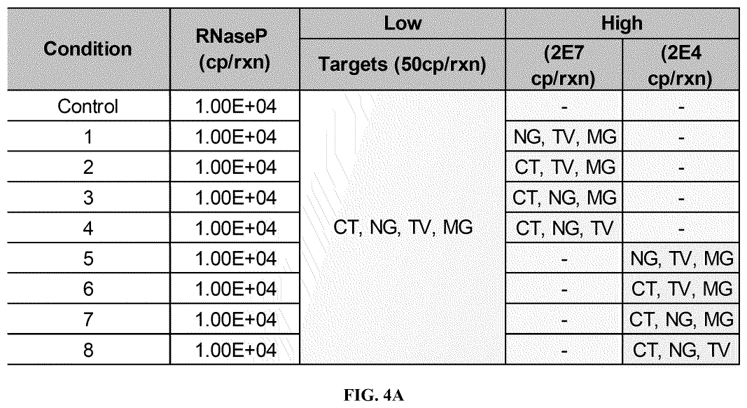

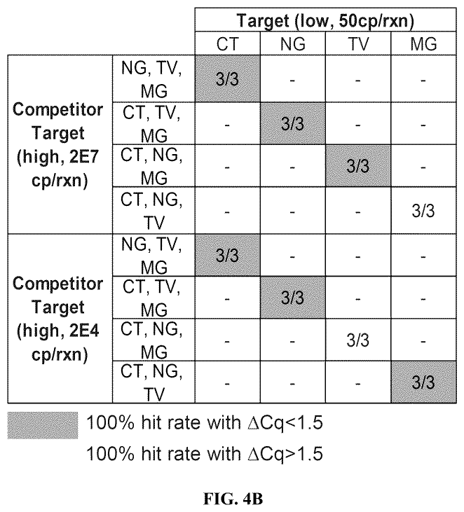

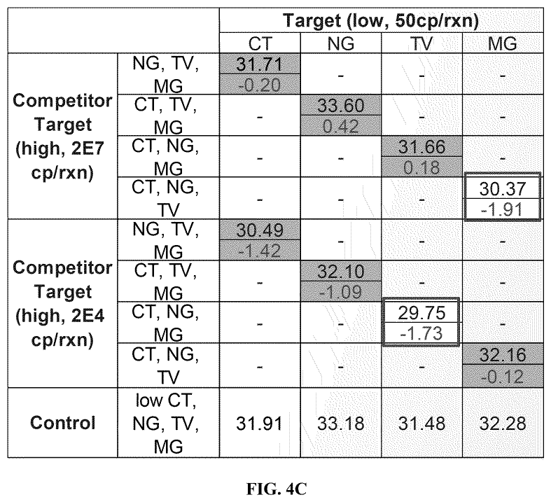

For the competitive interference studies, the assay's ability to detect low-concentration target template (50 cp/rxn) in the presence of a combination of high concentration target template (2E7 cp/rxn and 2E4 cp/rxn) was tested. The tested conditions are shown in FIG. 4A.

### Results

The STI pathogens CT, MG, NG, and TV organisms' single copy gene targets were used in the 5-plex assay. The targets demonstrated greater than 99% strain coverage, as shown in Table 12.

TABLE 12
A list of organisms and their single copy gene targets and strain coverage
as detected by one embodiment of the disclosed 5-plex assay.

| | | | Strain | |
|---|---|---|---|---|
| | Gene | Strain | coverage | |
| Organism | target | coverage | % | Specificity |
| Chlamydia | tarP | 442/442 | 100% | no off- |
| trachomatis | (translocated | | | targets |
| | actin recruiting | | | |
| | phosphoprotein) | | | |
| Mycoplasma | tRNA(Ile)- | 6/6 | 100% | no off- |
| genitalium | lysidine | | | targets |
| | synthase | | | |
| Neisseria | Restriction | 911/912 | 99.90% | no off- |
| gonorrhoeae | endonuclease | | | targets |
| | type I, HsdS, | | | |
| | NGO0357 | | | |
| Trichomonas | actin | 157/157 | 100% | 2 Histomonas |
| vaginalis | | | | meleagridis |
| | | | | actin fragments |
| | | | | (bird parasite) |

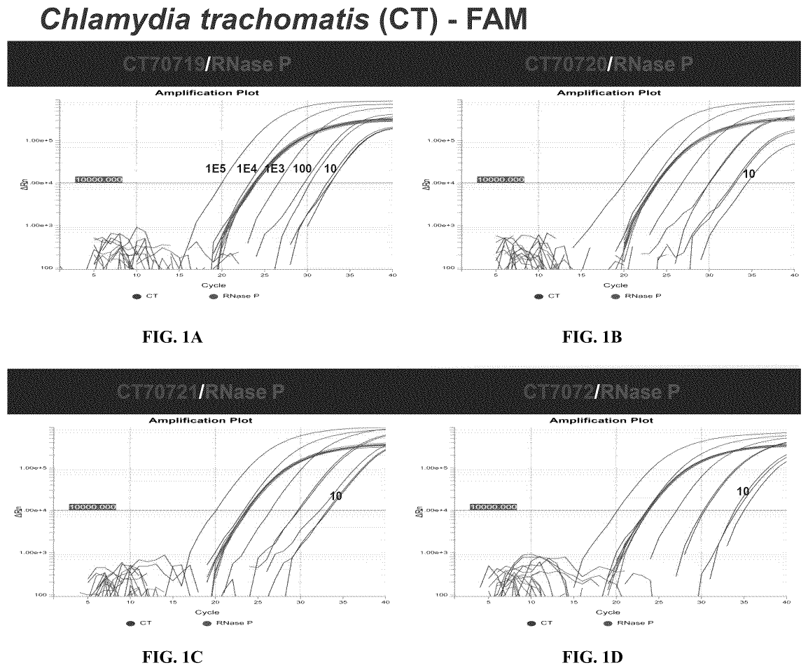

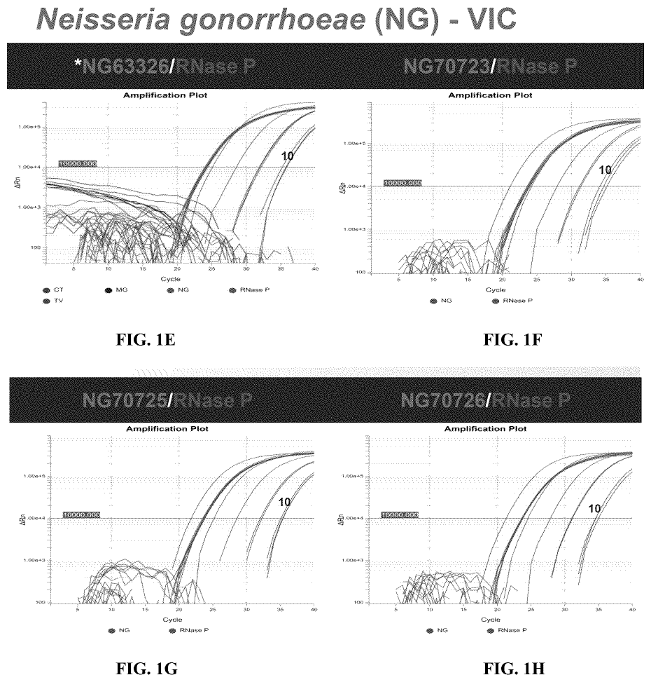

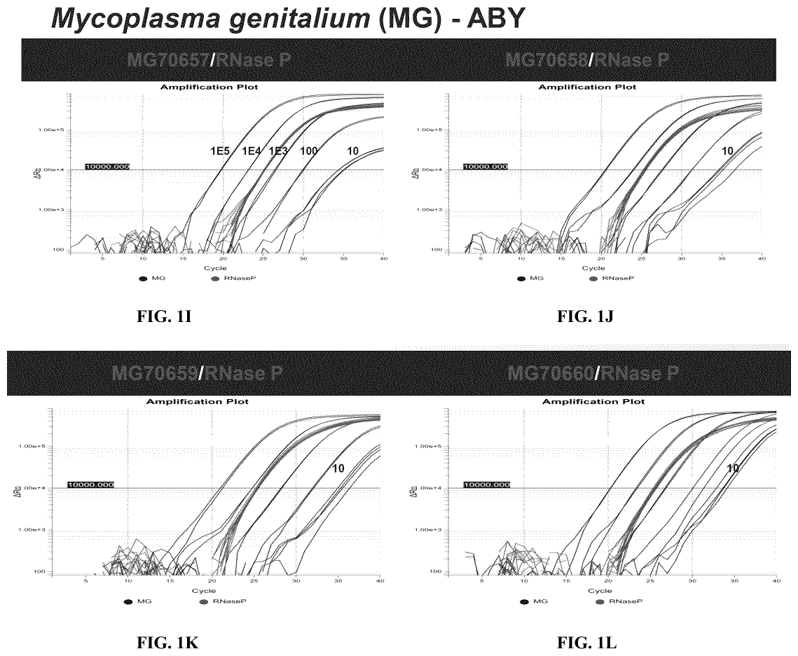

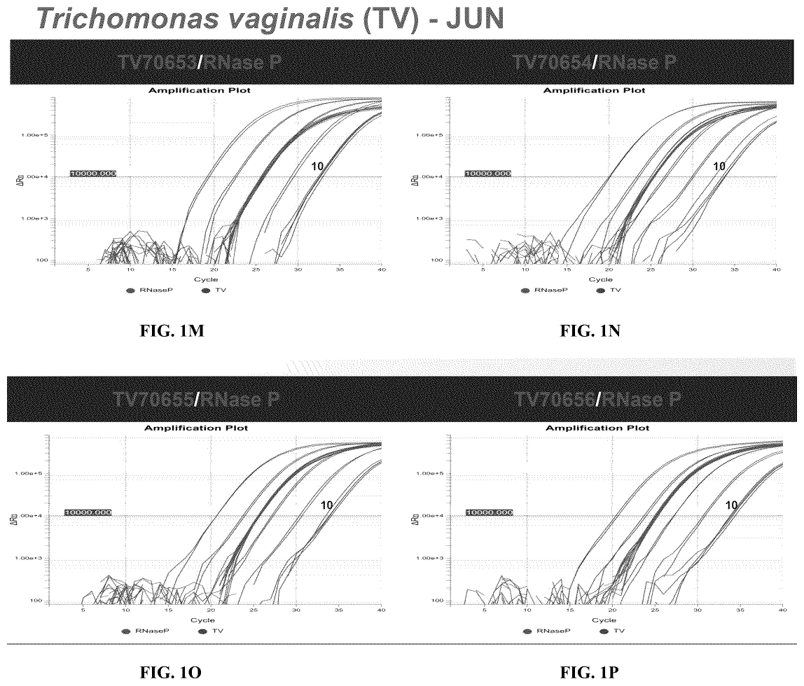

The amplification curves for the single genes are shown in FIGS. 1A-1P. The assay performance was confirmed both on synthetic DNA and on genomic DNA. The use of genomic DNA allowed to test whether there were any off-target effects, and off-target effects were not detected for the primers amplifying the CT, MG, and NG gene targets. Two off-target effects were observed with the primers amplifying the TG gene targets, and these included two Histomonas meleagridis actin fragments (bird parasite).

The results for assay linearity and PCR efficiency are shown in FIG. 2. The PCR efficiently was within 90-110% and the linearity was about 1 for all targets at all three threshold levels.

The developed 5-plex assay used the dye assignment described in Tables 4 and 5 and in Table 13, the primary assay panel references the primers and probes provided in Tables 2 and 3.

TABLE 13
The 5-plex assay components and primer and probe concentrations.

| | Primary | | Primer | Probe |
|---|---|---|---|---|
| | assay | | concen- | concen- |
| Target | panel | Dye | tration | tration |
| Chlamydia | See Tables | FAM ™ | 300 nM | 250 nM |
| trachomatis | 2 and 3 | | | |
| Neisseria | See Tables | VIC ™ | 300 nM | 250 nM |
| gonorrhoeae | 2 and 3 | | | |
| Mycoplasma | See Tables | ABY ™ | 300 nM | 250 nM |
| genitalium | 2 and 3 | | | |
| Trichomonas | See Tables | JUN ™ | 300 nM | 250 nM |
| vaginalis | 2 and 3 | | | |
| RNase P | See Tables | AF647 ™ | 300 nM | 150 nM |
| | 2 and 3 | | | |

The 5-plex assays used the TaqPath™ Infectious Disease 1-Step Multiplex Master Mix (No ROX). The master mix and the primers and probes were combined in a reaction mix as presented in Table 14 below.

TABLE 14
Reaction mix for the 5-plex assay.

| | Volume | Volume for |
|---|---|---|
| | per sample | n samples plus |
| Component | or control | 2 controls# |
| TrueMark ™ Infectious Disease | 6.25 μL | 6.25 × (1.2 × n) μL |
| 1-Step Multiplex Master Mix | | |
| (No ROX) | | |
| TrueMark ™ STI Select Panel | 1.25 μL | 1.25 × (1.2 × n) μL |
| Total reaction mix volume | 7.50 μL | |

#All volumes include 20% overage for pipette error

For each amplification reaction of the 5-plex assay, 7.5 μL of the reaction mix was pipetted into each amplification well, then 17.5 μL of either the extracted materials, the diluted amplification control, or nuclease-free water was added to designated wells.

The 5-plex assay reaction mixes with samples or controls were then subjected to amplification conditions as specified in Table 15.

TABLE 15
Amplification conditions for the 5-plex assay.

| | | | Number | Ramp |
|---|---|---|---|---|
| Step | Temperature | Time | of Cycles | Rate |
| UNG Incubation | 25° C. | 2 minutes | 1 | 1.6° C. |
| | | | | per second |
| Reverse | 53° C. | 10 minutes | 1 | 1.6° C. |
| transcription | | | | per second |
| Preincubation | 85° C. | 10 minutes | 1 | 1.6° C. |
| | | | | per second |
| Activation | 95° C. | 2 minutes | 1 | 1.6° C. |
| | | | | per second |
| Denaturation | 95° C. | 3 seconds | 40 | 1.6° C. |
| | | | | per second |
| Anneal/Extension | 60° C. | 32 seconds | | 1.6° C. |
| | | | | per second |

The target cutoffs specified in Table 16 were used for detection of the STI pathogens.

TABLE 16
Target cutoffs for detecting STI pathogens with the 5-plex assay.

| Target | Cq range |
|---|---|
| Chlamydia trachomatis (CT) | 30-36 |
| Neisseria gonorrhoeae (NG) | 30-35 |
| Mycoplasma genitalium (MG) | 30-35 |
| Trichomonas vaginalis (TV) | 31-35 |
| RNaseP (internal control) | 30-34 |

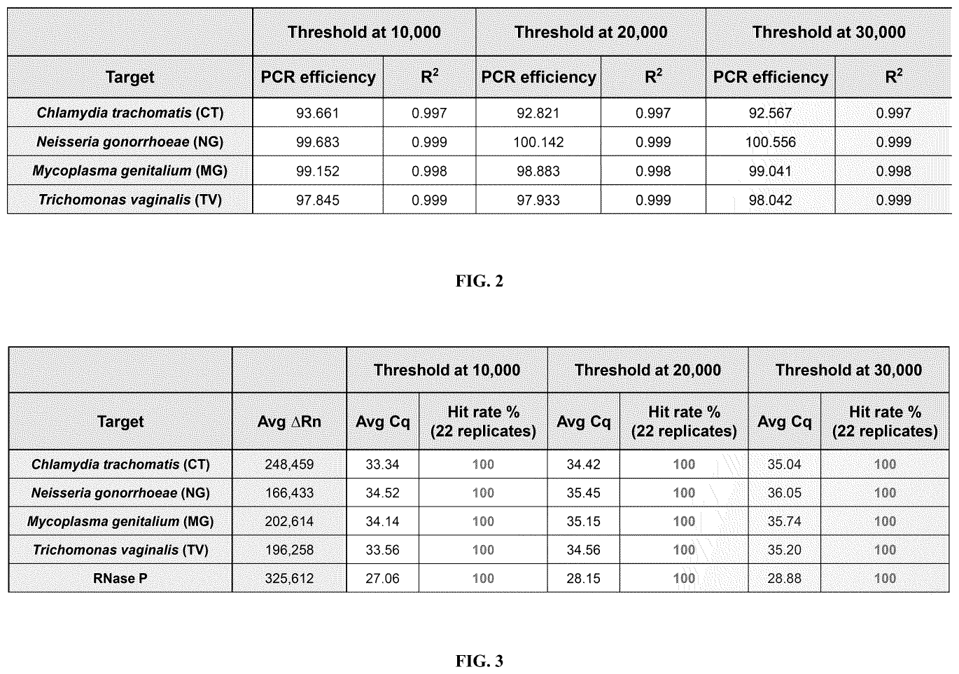

The analytical sensitivity of the assay was tested with the synthetic DNA. The assay demonstrated 100% detection of all 10 cp/rxn (copies per reaction) of the individual templates with good ΔRn (greater than 10⁵) and an average Cq of less than 36.1 (threshold at 30,000) (FIG. 3).

The assay was also tested for benchtop stability using synthetic DNA template concentration at 30 cp/rxn (3× analytical sensitivity) and incubation time at 0 h and 2 hrs post PCR set up for room temperature (RT) and 4° C. It was observed that the assay was stable at 4° C. for at least 2 hours after PCR set up. After 2 hrs incubation at 4° C. and RT, Cq remained consistent despite decrease in ΔRn. Overall Cq was less than 35 and ΔRn was greater than 1E5 at 30 cp/rxn.

The assay performance was also evaluated for competitive interferences in co-infection conditions. For STI, it is common to see co-infection with 2 pathogens. Incidence rate of co-infection with more than 3 pathogens is much lower and constitutes less than 1% of the infection cases (Getman et al., Journal of Clinical Microbiology, 54(9):2278-2283 (2016)).

Competitive interference study was designed to test the most stringent condition, which is co-infection of 4 pathogens, tested conditions are shown in FIG. 4A, and the results are shown in FIGS. 4B (showing the hit rate) and 4C (showing mean Cq and ΔCq summary). No significant competitive interference was observed, 100% low concentration target produced positive result in the presence of 2E7 cp/rxn and 2E4 cp/rxn competitors. Increased ΔCq was observed for MG and TV in the presence of high concentration competitors, but this did not affect accuracy of sample calling.

### Example 2. Assay Panel Performance with Clinical Samples

### Materials and Methods

The following specimens shown in Table 17 were used to evaluate the 5-plex assay performance with clinical samples.

TABLE 17
Specimens for evaluating the assay
performance with clinical samples.

| Type of Clinical Specimens | Matrix Type | # tested |
|---|---|---|
| Neisseria gonorrhoeae (NG+) | Genital Swabs | 7 |
| Trichomonas vaginalis (TV+) | Genital Swabs | 10 |
| Mycoplasma genitalium (MG+) | Genital Swabs | 10 |
| Chlamydia trachomatis (CT+) | Genital Swabs | 10 |
| Unknown (No result) | Genital Swab | 28 |
| ** Negative specimens | Genital Swabs | 62 |
| from Austin | | |
| (CT− and NG−) | | |
| Total specimens tested | | 127 |

### Results

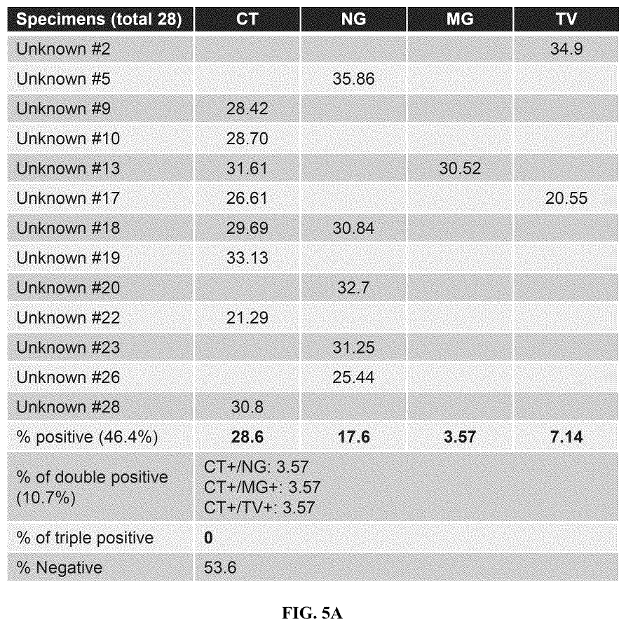

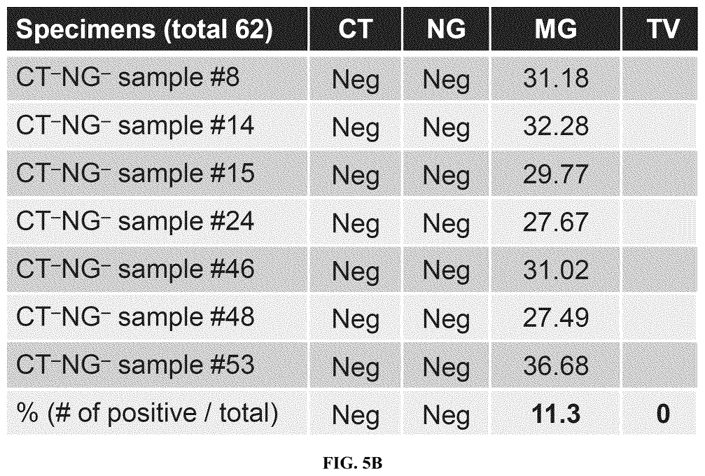

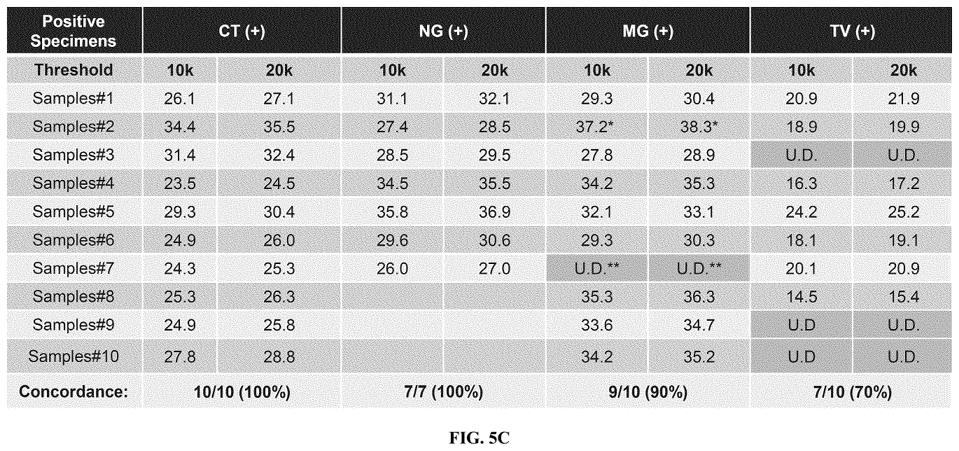

The results from the unknown clinical samples are shown in FIG. 5A. The results from CT− and NG− samples are shown in FIG. 5B. The 5-plex assay demonstrated 100% concordance for CT(−)/NG(−) specimens, 11.3% MG+ specimens were detected but no TV positive specimens for the CT(−)/NG(−) specimens. In unknown sample, CT+ and NG+ counted for 53.4%, while double positive was not very common. In the unknown clinical samples, 27% of clinical calls were positive for a double infection and 5.4% of clinical calls were positive for a triple infection.

The 5-plex assay showed equivalent concordance for positive confirmed specimens with 10 k and 20 k thresholds. The results are shown in FIG. 5C. A total 37 individual clinical samples had been confirmed positively by third party confirmation methods and tested with STI panel (10 samples for CT, MG and TV, and 7 for NG).

Therefore, it was observed that the 5-plex assay has:
1. Clean background with clinical specimens;
2. Reasonable amplification for positive specimens;
3. Able to detect different pathogens from co-infection and triple infections

### Example 3. Amplification Control

This study was performed to develop a robust and reliable amplification control that would be included as a positive control for detecting STI pathogens. The amplification control was developed and labeled as STI amplification control. The amplification control includes an all-in-one superplasmid, which has all the amplicons in the assay panel candidates.

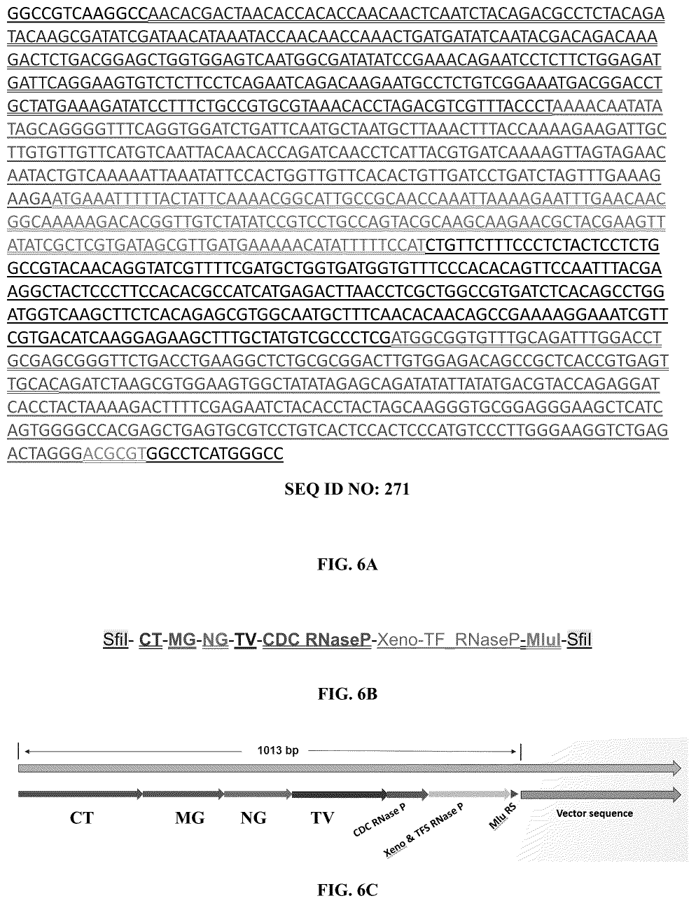

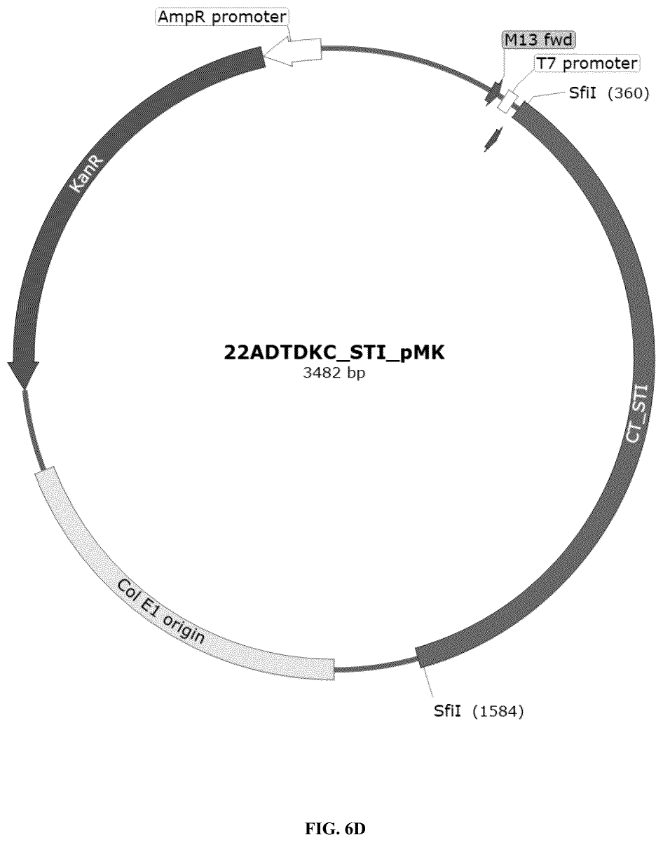

The sequence of the plasmid insert is provided as SEQ ID NO: 271 in Table 9 and in FIG. 6A. The different target sequences for the different STI pathogens are presented in tandem and are demarcated with single or double underlines. FIG. 6B provides the organization of the target sequences in the insert, which starts with the SfiI restriction enzyme target sequence, followed by target sequences for CT, MG, NG, TV, RNaseP, MluI, and ends with the SfiI restriction enzyme target sequence. This arrangement is also demarcated with single or double underlines and matches that of FIG. 6A. The tandem organization of the target sequences in the insert is also presented in FIG. 6C. The map of the plasmid (without the insert) is shown in FIG. 6D.

### Exemplary Embodiments

The following items are a list of exemplary embodiments. It should be understood that any part or parts of any item can be combined with any part or parts of any other items:
1. A composition for amplifying target sequences from sexually transmitted infection (STI) pathogen genomes at greater than 99% strain coverage for each pathogen, the composition comprising:
   (a) a first set of forward primers and reverse primers for amplifying one or more target sequences present in target regions of the Chlamydia trachomatis (CT) genome;
   (b) a second set of forward primers and reverse primers for amplifying one or more target sequences present in target regions of the Mycoplasma genitalium (MG) genome;
   (c) a third set of forward primers and reverse primers for amplifying one or more target sequences present in target regions of the Neisseria gonorrhoeae (NG) genome; and
   (d) a fourth set of forward primers and reverse primers for amplifying one or more target sequences present in target regions of the Trichomonas vaginalis (TV) genome,
   wherein the composition provides greater than 99% strain coverage for each pathogen.
2. The composition of item 1, wherein the first set of forward primers and reverse primers comprises:
   (a) a first forward primer and a first reverse primer configured to amplify a first target sequence present in a first target region of the CT genome, wherein the first target sequence includes at least 10 contiguous nucleotides of the first target region of the CT genome;
   (b) a second forward primer and a second reverse primer configured to amplify a second target sequence present in a second target region of the CT genome, wherein the second target sequence includes at least 10 contiguous nucleotides of the second target region of the CT genome;
   (c) a third forward primer and a third reverse primer configured to amplify a third target sequence present in a third target region of the CT genome, wherein the third target sequence includes at least 10 contiguous nucleotides of the third target region of the CT genome;
   (d) a fourth forward primer and a fourth reverse primer configured to amplify a fourth target sequence present in a fourth target region of the CT genome, wherein the fourth target sequence includes at least 10 contiguous nucleotides of the fourth target region of the CT genome; and/or
   (e) a fifth forward primer and a fifth reverse primer configured to amplify a fifth target sequence present in a fifth target region of the CT genome, wherein the fifth target sequence includes at least 10 contiguous nucleotides of the fifth target region of the CT genome.
3. The composition of item 1 or 2, wherein the second set of forward primers and reverse primers comprises:
   (a) a first forward primer and a first reverse primer configured to amplify a first target sequence present in a first target region of the MG genome, wherein the first target sequence includes at least 10 contiguous nucleotides of the first target region of the MG genome; and/or
   (b) a second forward primer and a second reverse primer configured to amplify a second target sequence present in a second target region of the MG genome, wherein the second target sequence includes at least 10 contiguous nucleotides of the second target region of the MG genome.
4. The composition of any one of items 1-3, wherein the third set of forward primers and reverse primers comprises:
   (a) a first forward primer and a first reverse primer configured to amplify a first target sequence present in a first target region of the NG genome, wherein the first target sequence includes at least 10 contiguous nucleotides of the first target region of the NG genome;
   (b) a second forward primer and a second reverse primer configured to amplify a second target sequence present in a second target region of the NG genome, wherein the second target sequence includes at least 10 contiguous nucleotides of the second target region of the NG genome;
   (c) a third forward primer and a third reverse primer configured to amplify a third target sequence present in a third target region of the NG genome, wherein the third target sequence includes at least 10 contiguous nucleotides of the third target region of the NG genome; and/or
   (d) a fourth forward primer and fourth third reverse primer configured to amplify a fourth target sequence present in a fourth target region of the NG genome, wherein the fourth target sequence includes at least 10 contiguous nucleotides of the fourth target region of the NG genome.
5. The composition of any one of items 1-4, wherein the fourth set of forward primers and reverse primers comprises:
   (a) a first forward primer and a first reverse primer configured to amplify a first target sequence present in a first target region of the TV genome, wherein the first target sequence includes at least 10 contiguous nucleotides of the first target region of the TV genome; and/or
   (b) a second forward primer and a second reverse primer configured to amplify a second target sequence present in a second target region of the TV genome, wherein the second target sequence includes at least 10 contiguous nucleotides of the second target region of the TV genome.
6. The composition of any one of items 1-5, wherein the composition further comprises a set of forward primers and reverse primers comprising:
   a first forward primer and a first reverse primer configured to amplify a first target sequence present in a first target region of a control gene, wherein the first target sequence includes at least 10 contiguous nucleotides of the control gene.
7. The composition of any one of items 1-6, wherein the target regions of the CT genome comprise regions within the tarP, BamD, Trp operon repressor, cysteine-rich outer membrane protein (CRPA), and/or the polymorphic outer membrane protein repeat-containing protein gene regions of CT.
8. The composition of any one of items 1-7, wherein the target regions of the CT genome are selected from SEQ ID NOs: 163-180.
9. The composition of any one of items 1-8, wherein the target regions of the MG genome comprise regions within the ComEC/Rec2-related protein gene and/or the tRNA(Ile)-lysidine synthetase gene regions of MG.
10. The composition of any one of items 1-9, wherein the target regions of the MG genome are selected from SEQ ID NOs: 181-189.
11. The composition of any one of items 1-10, wherein the target regions of the NG genome comprise regions within the NGO0357, the NGO_0357 hypothetical protein, the NGO_1110 phage protein, and/or the NGO_1117 phage protein gene regions of NG.
12. The composition of any one of items 1-11, wherein the target regions of the NG genome are selected from SEQ ID NOs: 190-205.
13. The composition of any one of items 1-12, wherein the target regions of the TV genome comprise regions within the alpha-tubulin-1 (α-TUB1) and/or the actin gene regions of TV.
14. The composition of any one of items 1-13, wherein the target regions of the TV genome are selected from SEQ ID NOs: 206-215.
15. The composition of any one of items 1-14, wherein the composition further comprises a fifth set of forward primers and reverse primers for amplifying a first target sequence present in a first target region of a control gene.
16. The composition of item 15, wherein the control gene is human RNaseP gene.
17. The composition of item 15 or 16, wherein the target region of the control gene comprises SEQ ID NO: 216.
18. The composition of any one of the preceding items, comprising: a nucleic acid sample, a polymerase, a buffer, and nucleotides.
19. The composition of any one of the preceding items, further comprising a first probe, a second probe, a third probe, a fourth probe, and/or a fifth probe.
20. The composition of item 19, wherein each of the first probe, the second probe, the third probe, the fourth probe, and/or the fifth probe comprises at least one label.
21. The composition of item 20, wherein the at least one label for the first probe comprises a first label, the at least one label for the second probe comprises a second label, the at least one label for the third probe comprises a third label, the at least one label for the fourth probe comprises a fourth label, and/or the at least one label for the fifth probe comprises a fifth label.
22. The composition of item 20 or 21, wherein the at least one label comprises a fluorescent or other detectable label.
23. The composition of item 21 or 22, wherein the first label, the second label, the third label, the fourth label, and/or the fifth label is a fluorescent label and each one of the first probe, the second probe, the third probe, the fourth probe, and/or the fifth probe further comprises a quencher or a minor groove-binder (MGB).
24. The composition of any one of items 19-23, wherein each of the first probe, the second probe, the third probe, the fourth probe, and/or the fifth probe comprises a different fluorescent label selected from FAM™, VIC™, ABY™, JUN™; FITC; 6-carboxy-4′,5′-dichloro-2′,7′-dimethoxy fluorescein (JOE™); 6-carboxy-1,4-dichloro-2′,7′-dichloro-fluorescein (TET™); 6-carboxy-1,4-dichloro-2′,4′,5′,7′-tetra-chlorofluorescein (HEX™); Alexa Fluor fluorophores, NED™, and BODIPY™ fluorophores.
25. The composition of item 24, wherein the quencher is selected from QSY® and BHQ™ dyes.
26. The composition of any one of the preceding items, wherein the first set of forward primers and reverse primers for amplifying the one or more target sequences present in the target regions of the CT genome are selected from SEQ ID NOs: 1-36.
27. The composition of any one of the preceding items, wherein the second set of forward primers and reverse primers for amplifying the one or more target sequences present in the target regions of the MG genome are selected from SEQ ID NOs: 37-52.
28. The composition of any one of the preceding items, wherein the third set of forward primers and reverse primers for amplifying the one or more target sequences present in the target regions of the NG genome are selected from SEQ ID NOs: 53-86.
29. The composition of any one of the preceding items, wherein the fourth set of forward primers and reverse primers for amplifying the one or more target sequences present in the target regions of the TV genome are selected from SEQ ID NOs: 87-106.
30. The composition of any one of items 15-29, wherein the fifth set of forward primers and reverse primers for amplifying the target sequence present in the target region of a control gene comprises SEQ ID NOs: 107 and 108.
31. The composition of any one of items 19-30, wherein the first probe, the second probe, the third probe, the fourth probe, and/or the fifth probe are probes for detecting the one or more target sequences present in the target regions of the CT, MG, NG, TV genomes, and/or the control gene.
32. The composition of item 31, wherein the probes are selected from SEQ ID NOs: 109-162.
33. The composition of any one of items 19-32, wherein the first probe is labeled with FAM™ fluorescent label, the second probe is labeled with ABY™ fluorescent label, the third probe is labeled with VIC™ fluorescent label, the fourth probe is labeled with JUN™ fluorescent label, and/or the fifth probe is labeled with Alexa Fluor™ AF647 fluorescent label.
34. The composition of any one of items 19-33, wherein the first probe has a sequence of any one of SEQ ID NOs: 109-126, the second probe has a sequence of any one of SEQ ID NOs: 127-134, the third probe has a sequence of any one of SEQ ID NOs: 135-151, the fourth probe has a sequence of any one of SEQ ID NOs: 152-161, and/or the fifth probe has a sequence of SEQ ID NO: 162.
35. The composition of any one of the preceding items, further comprising a positive control for amplification.
36. The composition of item 35, wherein the positive control is a synthetic plasmid comprising an insert sequence of SEQ ID NO: 271.
37. A kit, comprising: a set of forward primers and reverse primers of any one of the compositions of the preceding items.
38. The kit of item 37, further comprising a probe of the composition of any one of items 19-36.
39. A method for detecting CT, MG, NG, and/or TV in a biological sample, comprising:
   (a) combining a biological sample and a composition of any one of items 1-36 to form a reaction mixture;
   (b) subjecting the reaction mixture to amplification conditions, thereby forming one or more amplification products; and
   (c) detecting at least one of the amplification products.
40. The method of item 39, wherein the biological sample is selected from the group consisting of a blood sample, a buccal sample, a urine sample, a semen sample, a vaginal secretion sample, and a vaginal swab.
41. The method of item 39 or 40, wherein the biological sample is from a human.
42. The method of item 39 or 40, wherein the biological sample is from a non-human subject.

Appendix—TrueMark™ STI Select Panel, Combo Kit USER GUIDE—as published on 21 Dec. 2022, is incorporated herein by reference in its entirety.

What is claimed:

1. A composition for amplifying target sequences from sexually transmitted infection (STI) pathogen genomes at about 99% strain coverage for each pathogen, the composition comprising:
(a) a first set of forward primers and reverse primers for amplifying one or more target sequences present in target regions of the Chlamydia trachomatis (CT) genome;
(b) a second set of forward primers and reverse primers for amplifying one or more target sequences present in target regions of the Mycoplasma genitalium (MG) genome;
(c) a third set of forward primers and reverse primers for amplifying one or more target sequences present in target regions of the Neisseria gonorrhoeae (NG) genome; and
(d) a fourth set of forward primers and reverse primers for amplifying one or more target sequences present in target regions of the Trichomonas vaginalis (TV) genome,
wherein the composition provides about 99% strain coverage for each pathogen.

2. The composition of claim 1, wherein:
the first set of forward primers and reverse primers comprises:
(a) a first forward primer and a first reverse primer configured to amplify a first target sequence present in a first target region of the CT genome, wherein the first target sequence includes at least 10 contiguous nucleotides of the first target region of the CT genome;
(b) a second forward primer and a second reverse primer configured to amplify a second target sequence present in a second target region of the CT genome, wherein the second target sequence includes at least 10 contiguous nucleotides of the second target region of the CT genome;
(c) a third forward primer and a third reverse primer configured to amplify a third target sequence present in a third target region of the CT genome, wherein the third target sequence includes at least 10 contiguous nucleotides of the third target region of the CT genome;
(d) a fourth forward primer and a fourth reverse primer configured to amplify a fourth target sequence present in a fourth target region of the CT genome, wherein the fourth target sequence includes at least 10 contiguous nucleotides of the fourth target region of the CT genome; and/or
(e) a fifth forward primer and a fifth reverse primer configured to amplify a fifth target sequence present in a fifth target region of the CT genome, wherein the fifth target sequence includes at least 10 contiguous nucleotides of the fifth target region of the CT genome;
the second set of forward primers and reverse primers comprises:
(a) a first forward primer and a first reverse primer configured to amplify a first target sequence present in a first target region of the MG genome, wherein the first target sequence includes at least 10 contiguous nucleotides of the first target region of the MG genome; and/or
(b) a second forward primer and a second reverse primer configured to amplify a second target sequence present in a second target region of the MG genome, wherein the second target sequence includes at least 10 contiguous nucleotides of the second target region of the MG genome;
the third set of forward primers and reverse primers comprises:
(a) a first forward primer and a first reverse primer configured to amplify a first target sequence present in a first target region of the NG genome, wherein the first target sequence includes at least 10 contiguous nucleotides of the first target region of the NG genome;
(b) a second forward primer and a second reverse primer configured to amplify a second target sequence present in a second target region of the NG genome, wherein the second target sequence includes at least 10 contiguous nucleotides of the second target region of the NG genome;
(c) a third forward primer and a third reverse primer configured to amplify a third target sequence present in a third target region of the NG genome, wherein the third target sequence includes at least 10 contiguous nucleotides of the third target region of the NG genome; and/or
(d) a fourth forward primer and fourth third reverse primer configured to amplify a fourth target sequence present in a fourth target region of the NG genome, wherein the fourth target sequence includes at least 10 contiguous nucleotides of the fourth target region of the NG genome; and
the fourth set of forward primers and reverse primers comprises:
(a) a first forward primer and a first reverse primer configured to amplify a first target sequence present in a first target region of the TV genome, wherein the first target sequence includes at least 10 contiguous nucleotides of the first target region of the TV genome; and/or
(b) a second forward primer and a second reverse primer configured to amplify a second target sequence present in a second target region of the TV genome, wherein the second target sequence includes at least 10 contiguous nucleotides of the second target region of the TV genome.

3. The composition of claim 1, wherein
the target regions of the CT genome comprise regions within the tarP, BamD, Trp operon repressor, cysteine-rich outer membrane protein (CRPA), and/or the polymorphic outer membrane protein repeat-containing protein gene regions of CT;
the target regions of the MG genome comprise regions within the ComEC/Rec2-related protein gene and/or the tRNA(Ile)-lysidine synthetase gene regions of MG;
the target regions of the NG genome comprise regions within the NGO0357, the NGO_0357 hypothetical protein, the NGO_1110 phage protein, and/or the NGO_1117 phage protein gene regions of NG; and
the target regions of the TV genome comprise regions within the alpha-tubulin-1 (α-TUB1) and/or the actin gene regions of TV.

4. The composition of claim 1, wherein
the target regions of the CT genome are selected from SEQ ID NOs: 163-180;
the target regions of the MG genome are selected from SEQ ID NOs: 181-189;
the target regions of the NG genome are selected from SEQ ID NOs: 190-205; and
the target regions of the TV genome are selected from SEQ ID NOs: 206-215.

5. The composition of claim 1, wherein the composition further comprises a fifth set of forward primers and reverse primers for amplifying a first target sequence present in a first target region of a control gene.

6. The composition of claim 5, wherein the control gene is human RNaseP gene.

7. The composition of claim 5, wherein the target region of the control gene comprises SEQ ID NO: 216.

8. The composition of claim 1, further comprising a first probe, a second probe, a third probe, a fourth probe, and/or a fifth probe.

9. The composition of claim 8, wherein each of the first probe, the second probe, the third probe, the fourth probe, and/or the fifth probe comprises at least one label.

10. The composition of claim 9, wherein the at least one label for the first probe comprises a first label, the at least one label for the second probe comprises a second label, the at least one label for the third probe comprises a third label, the at least one label for the fourth probe comprises a fourth label, and/or the at least one label for the fifth probe comprises a fifth label.

11. The composition of claim 10, wherein the first label, the second label, the third label, the fourth label, and/or the fifth label is a fluorescent label or other detectable label and each one of the first probe, the second probe, the third probe, the fourth probe, and/or the fifth probe further comprises a quencher or a minor groove-binder (MGB).

12. The composition of claim 11, wherein each of the first probe, the second probe, the third probe, the fourth probe, and/or the fifth probe comprises a different fluorescent label selected from FAM™, VIC™, ABY™, JUN™; FITC; 6-carboxy-4′,5′-dichloro-2′,7′-dimethoxy-fluorescein (JOE™); 6-carboxy-1,4-dichloro-2′,7′-dichloro-fluorescein (TET™); 6-carboxy-1,4-dichloro-2′,4′,5′,7′-tetra-chlorofluorescein (HEX™); Alexa Fluor fluorophores, NED™, and BODIPY™ fluorophores, and
wherein the quencher is selected from QSY® and BHQ™ dyes.

13. The composition of claim 8, wherein the first probe, the second probe, the third probe, the fourth probe, and/or the fifth probe are probes for detecting the one or more target sequences present in the target regions of the CT, MG, NG, TV genomes, and/or the control gene; and wherein:
the first set of forward primers and reverse primers for amplifying the one or more target sequences present in the target regions of the CT genome are selected from SEQ ID NOs: 1-36;
the second set of forward primers and reverse primers for amplifying the one or more target sequences present in the target regions of the MG genome are selected from SEQ ID NOs: 37-52;
the third set of forward primers and reverse primers for amplifying the one or more target sequences present in the target regions of the NG genome are selected from SEQ ID NOS: 53-86;
the fourth set of forward primers and reverse primers for amplifying the one or more target sequences present in the target regions of the TV genome are selected from SEQ ID NOs: 87-106;
the fifth set of forward primers and reverse primers for amplifying the target sequence present in the target region of a control gene comprises SEQ ID NOs: 107 and 108; and
the probes are selected from SEQ ID NOs: 109-162, wherein
the first probe has a sequence of any one of SEQ ID NOs: 109-126,
the second probe has a sequence of any one of SEQ ID NOs: 127-134,
the third probe has a sequence of any one of SEQ ID NOs: 135-151,
the fourth probe has a sequence of any one of SEQ ID NOs: 152-161, and/or
the fifth probe has a sequence of SEQ ID NO: 162.

14. The composition of claim 8, wherein the first probe is labeled with FAM™ fluorescent label, the second probe is labeled with ABY™ fluorescent label, the third probe is labeled with VIC™ fluorescent label, the fourth probe is labeled with JUN™ fluorescent label, and/or the fifth probe is labeled with Alexa Fluor™ AF647 fluorescent label.

15. The composition of claim 1, comprising a positive control for amplification, a nucleic acid sample, a polymerase, a buffer, and/or nucleotides.

16. The composition of claim 15, wherein the positive control is a synthetic plasmid comprising an insert sequence of SEQ ID NO: 271.

17. A kit, comprising: a set of forward primers and reverse primers of claim 1 and a first probe, a second probe, a third probe, a fourth probe, and/or a fifth probe for detecting one or more target sequences present in target regions of Chlamydia trachomatis (CT), Mycoplasma genitalium (MG), Neisseria gonorrhoeae (NG), Trichomonas vaginalis (TV) genomes, and/or the control gene.

18. A method for detecting Chlamydia trachomatis (CT), Mycoplasma genitalium (MG), Neisseria gonorrhoeae (NG), and/or Trichomonas vaginalis (TV) in a biological sample, comprising:
(a) combining a biological sample and a composition of claim 1 to form a reaction mixture;
(b) subjecting the reaction mixture to amplification conditions, thereby forming one or more amplification products; and
(c) detecting at least one of the amplification products.

19. The method of claim 18, wherein the biological sample is selected from the group consisting of a blood sample, a buccal sample, a urine sample, a semen sample, a vaginal secretion sample, and a vaginal swab.

20. The method of claim 18, wherein the biological sample is from a human or a non-human subject.
